(19) United States
(12) Patent Application Publication
(10) Pub. No.: US 2024/0076292 A1
(43) Pub. Date: Mar. 7, 2024

(54) SULFONE DERIVATIVE PRODUCTION METHOD

(71) Applicant: KUMIAI CHEMICAL INDUSTRY CO., LTD., Tokyo (JP)

(72) Inventors: Shinki TANI, Tokyo (JP); Daisuke SHIKAMA, Tokyo (JP); Kai TAKIZAWA, Tokyo (JP)

(73) Assignee: KUMIAI CHEMICAL INDUSTRY CO., LTD., Tokyo (JP)

(21) Appl. No.: 18/258,906

(22) PCT Filed: Dec. 22, 2021
(86) PCT No.: PCT/JP2021/047734

§ 371 Date: Jun. 22, 2023

(30) Foreign Application Priority Data
Dec. 23, 2020 (WO) PCT/JP2020/048082

(51) Int. Cl. C07D 413/12

(52) U.S. Cl. CPC C07D 413/12

(57) ABSTRACT

The present invention provides an industrially desirable production method for a sulfone derivative that is useful as a herbicide, and an intermediate thereof.

### TECHNICAL FIELD

The present invention relates to a process for producing a sulfone derivative useful as a herbicide, that is, a compound of the following formula (8):

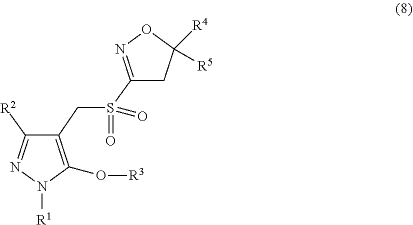

wherein R¹, R², R³, R⁴ and R⁵ are as described herein.

### BACKGROUND ART

It is known that sulfone derivatives of the above formula (8) have a herbicidal activity as disclosed in WO 2002/062770 A1 (Patent Document 1). Among them, a compound of the formula (8-a) (pyroxasulfone) is well known as a superior herbicide.

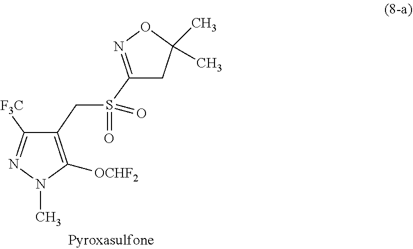 [SMILES: Cn1nc(C(F)(F)F)c(CS(=O)(=O)C2=NOC(C)(C)C2)c1OC(F)F]

As a process for producing the compound of the formula (8), a process by the oxidation of a sulfide derivative, i.e., a compound of the following formula (7) has been known, which is shown below.

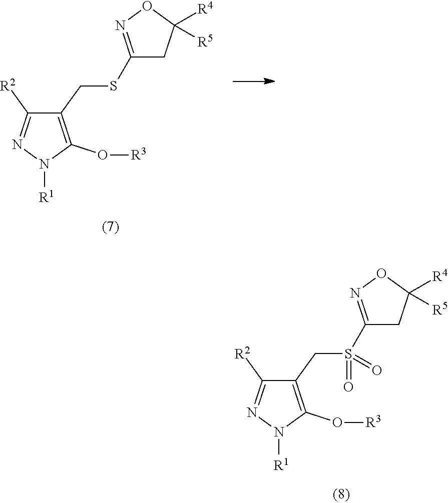

As shown in the following scheme, in Reference Example 3 in WO 2004/013106 A1 (Patent Document 2) is disclosed a process for producing 3-(5-difluoromethoxy-1-methyl-3-trifluoromethyl-1H-pyrazol-4-ylmethanesulfonyl)-5,5-dimethyl-2-isoxazoline (8-a) (pyroxasulfone) by oxidizing 3-(5-difluoromethoxy-1-methyl-3-trifluoromethyl-1H-pyrazol-4-ylmethylthio)-5,5-dimethyl-2-isoxazoline (7-a) (ISFP) with m-chloroperoxybenzoic acid (mCPBA).

### WO2004/013106A1. Reference Example 3

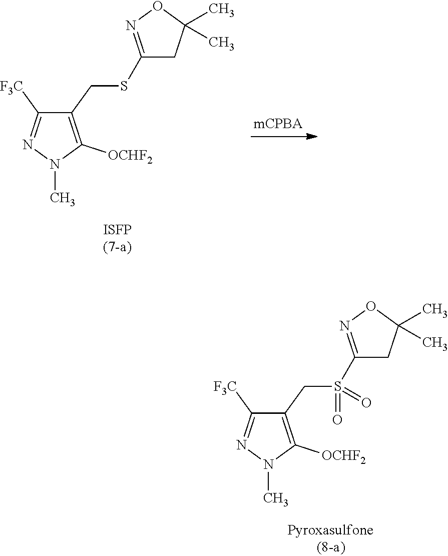 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

In a process for producing the compound of the formula (8) from the compound of the formula (7), m-chloroperbenzoic acid (mCPBA) described in WO 2004/013106 A1 (Patent Document 2) is expensive for industrial use, and in addition, has a problem of handling and waste. Therefore, the process for producing described in WO 2004/013106 A1 (Patent Document 2) is not practical for production on an industrial scale.

In addition, in the process for producing the compound of the formula (8) (sulfone derivative: SO₂ derivative) from the compound of the formula (7) (sulfide derivative: S derivative), there is a possibility that the reaction stops at a sulfoxide derivative (SO derivative) that is an intermediate of the oxidation reaction, i.e., a compound of the following formula (9):

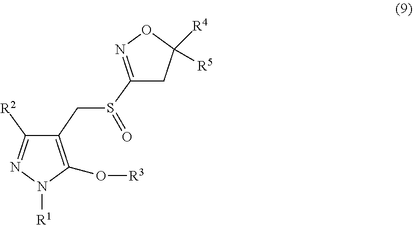

wherein R¹, R², R³, R⁴ and R⁵ are as described herein. Therefore, the compound of the formula (9) sometimes remains in the product as a by-product. The compound of the formula (9) that has contaminated a product such as a herbicide leads to the possibility of reduced quality and crop injury. However, the physical and chemical properties of the compound of the formula (9) are very similar to those of the compound of the formula (8), so that it is difficult to separate the compound of the formula (9) to purify the compound of the formula (8). Accordingly, regarding the process for producing the compound of the formula (8) from the compound of the formula (7), there has been desired a production process in which the oxidation reaction sufficiently proceeds and the amount of the compound of the formula (9) in the product is sufficiently small.

WO 2021/002484 A9 (Patent Document 9) describes a process for producing pyroxasulfone. This process is a superior process that has solved the above-described problems. On the other hand, there is still room for improvement in this process because a transition metal is used.

CN 111574511 A (Patent Document 10) describes a production process not using a transition metal in Example 4. The yield described therein is, however, low, and the process is lack of reproducibility.

### CITATION LIST

### Patent Document

Patent Document 1: WO 2002/062770 A1
Patent Document 2: WO 2004/013106 A1
Patent Document 3: WO 2005/095352 A1
Patent Document 4: WO 2005/105755 A1
Patent Document 5: WO 2007/094225 A1
Patent Document 6: WO 2006/068092 A1
Patent Document 7: JP 2013-512201 A
Patent Document 8: WO 2019/131715 A1
Patent Document 9: WO 2021/002484
Patent Document 10: CN 111574511 A

### SUMMARY OF INVENTION

### Technical Problem

It is an object of the present invention to provide a process for producing a compound of the formula (8) from a compound of the formula (7), that is, an industrially favorable production process in which the ratio of a compound of the formula (9) in a product is sufficiently low, and an excellent yield is obtained, and which is advantageous for production on an industrial scale.

It is another object of the present invention to provide an environmentally friendly process for producing a compound of the formula (8).

### Solution to Problem

As a result of earnest study, the present inventors have found that a compound of the formula (8) can be efficiently produced by reacting a compound of the formula (7) with an oxidizing agent by an oxidization method not using a transition metal as a catalyst as shown in the following step ii. Based on this finding, the present inventors have accomplished the present invention.

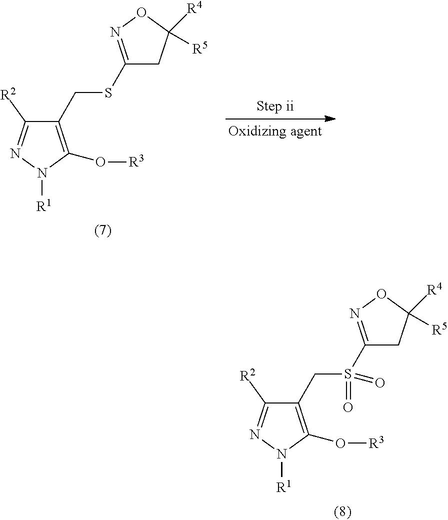

wherein R¹, R², R³, R⁴ and R⁵ are as described herein.

The present inventors have further found that an oxidation reaction can be caused to sufficiently proceed by performing, in the process for producing the compound of the formula (8) from the compound of the formula (7), a reaction with an oxidizing agent (preferably hydrogen peroxide, or an alkali metal persulfate, an ammonium persulfate salt or an alkali metal hydrogen persulfate, and more preferably hydrogen peroxide) under specific conditions. Based on this finding, the present inventors have accomplished a production process in which the amount of a compound of the formula (9) in a product is sufficiently small.

### Advantageous Effects of Invention

The present invention provides a novel process for producing a compound of the formula (8) which is excellent in the yield, and is environmentally friendly because no transition metal is used therein. Accordingly, the present invention contributes to sustainability.

The present invention also provides a process for producing a compound of the formula (8) (sulfone derivative: SO₂ derivative) from a compound of the formula (7) (sulfide derivative: S derivative), in which the ratio of a compound of the formula (9) (sulfoxide derivative: SO derivative) in a product is sufficiently low, and which is excellent in the yield, and is advantageous for production on an industrial scale. In the compound of the formula (8) produced by the process of the present invention, the amount of the compound of the formula (9), which can be a cause of reduced quality as a herbicide and crop injury, is sufficiently small, and hence this compound is useful as a herbicide.

The process of the present invention can be implemented on a large scale using low-cost materials, and is superior in economic efficiency, and is suitable for production on an industrial scale.

### DESCRIPTION OF EMBODIMENTS

In one aspect, the present invention is as follows:

[I-1] A process for producing a compound of the formula (8), comprising the following step ii:
(step ii) reacting a compound of the formula (7) with an oxidizing agent in the absence of a transition metal to produce the compound of the formula (8):

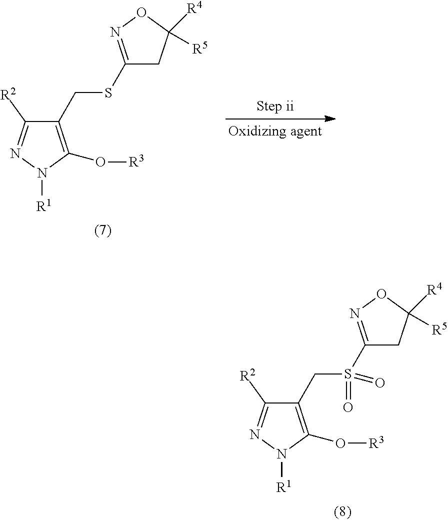

wherein R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents.
   [I-2] A process for producing a compound of the formula (8), comprising the following step i-a and step ii:
(step i-a) reacting a compound of the formula (1) with a compound of the formula (2) in the presence of a base to produce a compound of the formula (7):

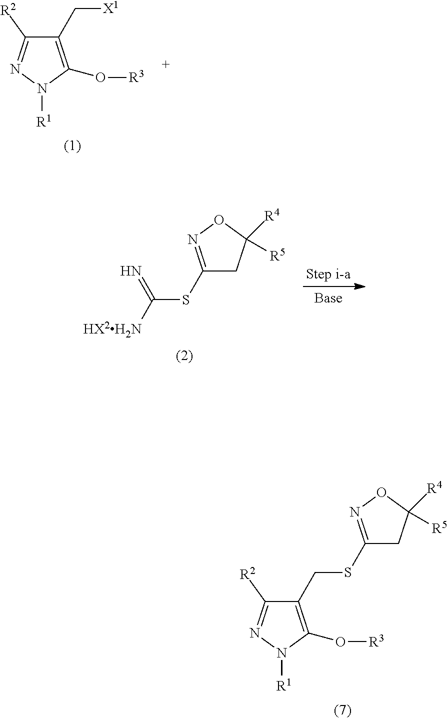

wherein in the formula (1), the formula (2), and the formula (7), R¹, R², R³, R⁴ and R⁵ are as defined above, X¹ is a leaving group, and X² is an atom or an atomic group forming an acid; and
(step ii) reacting the compound of the formula (7) with an oxidizing agent in the absence of a transition metal to produce the compound of the formula (8):

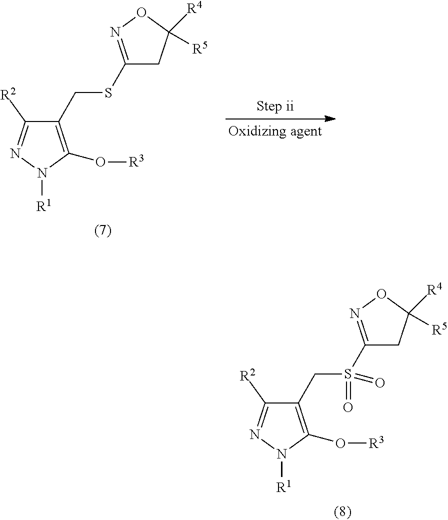

wherein in the formula (7) and the formula (8), R¹, R², R³, R⁴, and R⁵ are as defined above.
   [I-3] A process for producing a compound of the formula (8), comprising the following step i-b and step ii:
(step i-b) reacting a compound of the formula (4) with a compound of the formula (3) in the presence of a base to produce a compound of the formula (7):

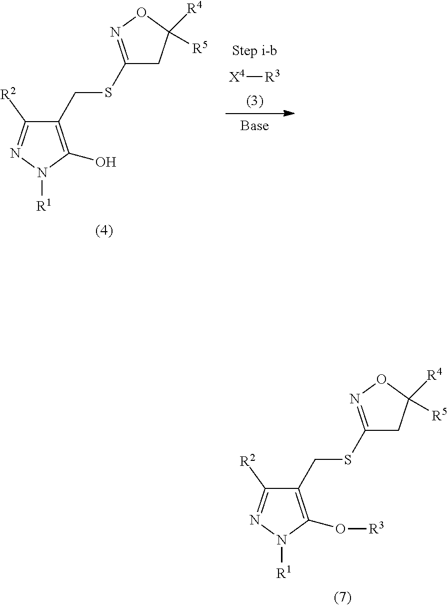

wherein in the formula (3), the formula (4) and the formula (7), R¹, R², R³, R⁴ and R⁵ are as defined above, and X⁴ is a leaving group; and
(step ii) reacting the compound of the formula (7) with an oxidizing agent in the absence of a transition metal to produce the compound of the formula (8):

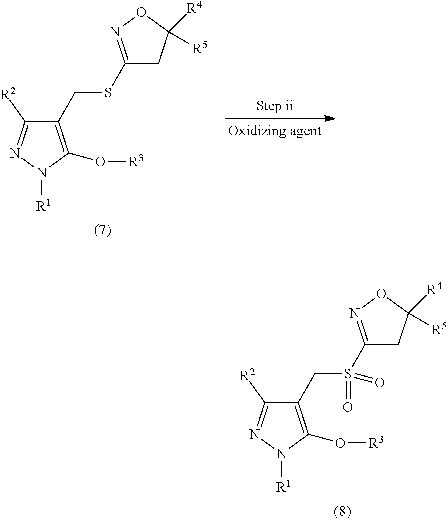

wherein in the formula (7) and the formula (8), R¹, R², R³, R⁴ and R⁵ are as defined above.
   [I-4] A process for producing a compound of the formula (8), comprising the following step i-c and step ii:
(step i-c) reacting a compound of the formula (5) with a compound of the formula (6) in the presence of a base to produce a compound of the formula (7):

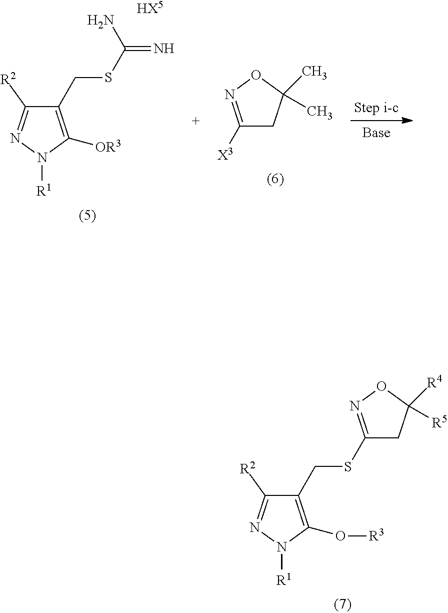

wherein in the formula (5), the formula (6) and the formula (7), R¹, R², R³, R⁴ and R⁵ are as defined above, X³ is a leaving group, and X⁵ is an atom or an atomic group forming an acid; and
(step ii) reacting the compound of the formula (7) with an oxidizing agent in the absence of a transition metal to produce the compound of the formula (8):

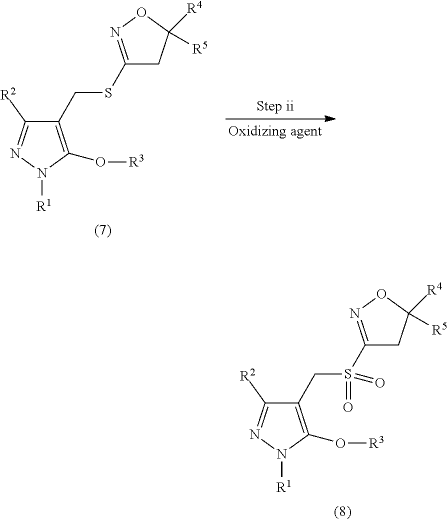

wherein in the formula (7) and the formula (8), R¹, R², R³, R⁴ and R⁵ are as defined above.
   [I-5] The process according to any one of [I-1] to [I-4], wherein the reaction in the step ii is performed in the presence of an acidic compound.
   [I-6] The process according to [I-5], wherein the acidic compound in the step ii is selected from mineral acids and carboxylic acids.
   [I-7] The process according to [I-5], wherein the acidic compound in the step ii is selected from sulfuric acid, acetic acid, and trifluoroacetic acid.
   [I-8] The process according to [I-5], wherein the acidic compound in the step ii is selected from sulfuric acid, sodium hydrogen sulfate, potassium hydrogen sulfate, acetic acid, and trifluoroacetic acid.
   [I-9] The process according to [I-5], wherein the acidic compound in the step ii is selected from sulfuric acid, potassium hydrogen sulfate, acetic acid, and trifluoroacetic acid.
   [I-10] The process according to [I-5], wherein the acidic compound in the step ii is sulfuric acid.
   [I-11] The process according to [I-5], wherein the acidic compound in the step ii is a (C1-C4)alkanoic acid.
   [I-12] The process according to [I-5], wherein the acidic compound in the step ii is acetic acid.
   [I-13] The process according to [I-5], wherein the acidic compound in the step ii is a (C2-C4)alkanoic acid substituted with 1 to 7 fluorine atoms.
   [I-14] The process according to [I-5], wherein the acidic compound in the step ii is trifluoroacetic acid.
   [I-15] The process according to any one of [I-5] to [I-14], wherein the amount of the acidic compound used in the step ii is larger than 0.10 mol based on 1 mol of the compound of the formula (7).
   [I-16] The process according to any one of [I-5] to [I-14], wherein the amount of the acidic compound used in the step ii is 0.5 mol or more based on 1 mol of the compound of the formula (7).
   [I-17] The process according to any one of [I-5] to [I-14], wherein the amount of the acidic compound used in the step ii is 1 mol or more based on 1 mol of the compound of the formula (7).
   [I-18] The process according to any one of [I-5] to [I-14], wherein the amount of the acidic compound used in the step ii is 2 mol or more based on 1 mol of the compound of the formula (7).
   [I-19] The process according to any one of [I-5] to [I-14], wherein the amount of the acidic compound used in the step ii is 100 mol or less based on 1 mol of the compound of the formula (7).
   [I-20] The process according to any one of [I-5] to [I-14], wherein the amount of the acidic compound used in the step ii is 50 mol or less based on 1 mol of the compound of the formula (7).
   [I-21] The process according to any one of [I-5] to [I-14], wherein the amount of the acidic compound used in the step ii is 30 mol or less based on 1 mol of the compound of the formula (7).
   [I-22] The process according to any one of [I-1] to [I-21], wherein the reaction in the step ii is performed in the presence of an organic solvent.
   [I-23] The process according to [I-22], wherein the organic solvent in the reaction in the step ii is selected from aromatic hydrocarbon derivatives, halogenated aliphatic hydrocarbons, alcohols, carboxylic acids, nitriles, carboxylic acid esters, ethers, ketones, amides, ureas, and sulfones.
   [I-24] The process according to [I-22], wherein the organic solvent in the reaction in the step ii is one or more (preferably one or two, and more preferably one) organic solvents selected from aromatic hydrocarbon derivatives, halogenated aliphatic hydrocarbons, alcohols, carboxylic acids, nitriles, carboxylic acid esters, ethers, ketones, amides, ureas, and sulfones.
   [I-25] The process according to [I-22], wherein the organic solvent in the reaction in the step ii is selected from aromatic hydrocarbon derivatives, carboxylic acids, alcohols, and nitriles.
   [I-26] The process according to [I-22], wherein the organic solvent in the reaction in the step ii is one or more (preferably one or two, and more preferably one) organic solvents selected from aromatic hydrocarbon derivatives, carboxylic acids, alcohols, and nitriles.
   [I-27] The process according to [I-22], wherein the organic solvent in the reaction in the step ii is selected from carboxylic acids, alcohols, and nitriles.
   [I-28] The process according to [I-22], wherein the organic solvent in the reaction in the step ii is one or two (preferably one) organic solvents selected from carboxylic acids, alcohols, and nitriles.
   [I-29] The process according to [I-22], wherein the organic solvent in the reaction in the step ii is one or more organic solvents selected from acetic acid, methanol, and acetonitrile.
   [I-30] The process according to [I-22], wherein the organic solvent in the reaction in the step ii is one or two (preferably one) organic solvents selected from acetic acid, methanol, and acetonitrile.
   [I-31] The process according to [I-22], wherein the organic solvent in the reaction in the step ii is an organic solvent having a relative permittivity of 1 to 40.
   [I-32] The process according to [I-22], wherein the organic solvent in the reaction in the step ii is an organic solvent having a Rohrschneider's polarity parameter of 1 to 7.
   [I-33] The process according to [I-22], wherein the organic solvent in the reaction in the step ii is an organic solvent having an acceptor number of 5 to 25.
   [I-34] The process according to [I-22], wherein the organic solvent in the reaction in the step ii is an organic solvent other than an alcohol.
   [I-35] The process according to [I-22], wherein the organic solvent in the reaction in the step ii is an organic solvent other than a (C1-C6)alcohol.
   [I-36] The process according to [I-22], wherein the organic solvent in the reaction in the step ii is an organic solvent other than a (C1-C4)alcohol.
   [I-37] The process according to [I-22], wherein the organic solvent in the reaction in the step ii is selected from aromatic hydrocarbon derivatives, nitriles, carboxylic acid esters, and amides.
   [I-38] The process according to [I-22], wherein the organic solvent in the reaction in the step ii is one or more (preferably one or two, and more preferably one) organic solvents selected from aromatic hydrocarbon derivatives, nitriles, carboxylic acid esters, and amides.
   [I-39] The process according to [I-22], wherein the organic solvent in the reaction in the step ii is selected from aromatic hydrocarbon derivatives, nitriles, and carboxylic acid esters.
   [I-40] The process according to [I-22], wherein the organic solvent in the reaction in the step ii is one or more (preferably one or two, and more preferably one) organic solvents selected from aromatic hydrocarbon derivatives, nitriles, and carboxylic acid esters.
   [I-41] The process according to [I-22], wherein the organic solvent in the reaction in the step ii is selected from benzene optionally substituted with one to three (preferably one or two, and more preferably one) selected from (C1-C4)alkyl groups and a chlorine atom, (C2-C5)alkane nitriles, (C1-C4)alkyl (C1-C6)carboxylates, N,N-di((C1-C4)alkyl) (C1-C4)alkaneamides, and 1-(C1-C4)alkyl-2-pyrrolidone.
   [I-42] The process according to [I-22], wherein the organic solvent in the reaction in the step ii is one or more (preferably one or two, and more preferably one) organic solvents selected from benzene optionally substituted with one to three (preferably one or two, and more preferably one) selected from (C1-C4)alkyl groups and a chlorine atom, (C2-C5)alkane nitriles, (C1-C4)alkyl (C1-C6)carboxylates, N,N-di((C1-C4)alkyl) (C1-C4)alkaneamides, and 1-(C1-C4)alkyl-2-pyrrolidone.
   [I-43] The process according to [I-22], wherein the organic solvent in the reaction in the step ii is selected from benzene optionally substituted with one to three (preferably one or two, and more preferably one) selected from (C1-C4)alkyl groups and a chlorine atom, (C2-C5)alkane nitriles, and (C1-C4)alkyl (C1-C6)carboxylates.
   [I-44] The process according to [I-22], wherein the organic solvent in the reaction in the step ii is one or more (preferably one or two, and more preferably one) organic solvents selected from benzene optionally substituted with one to three (preferably one or two, and more preferably one) selected from (C1-C4)alkyl groups and a chlorine atom, (C2-C5)alkane nitriles, and (C1-C4)alkyl (C1-C6)carboxylates.
   [I-45] The process according to [I-22], wherein the organic solvent in the reaction in the step ii is selected from toluene, xylene, chlorobenzene, dichlorobenzene, acetonitrile, methyl acetate, ethyl acetate, propyl acetate, isopropyl acetate, butyl acetate and isomers thereof, pentyl acetate and isomers thereof, hexyl acetate and isomers thereof, N,N-dimethylformamide (DMF), N,N-dimethylacetamide (DMAC), N,N-diethylacetamide, and N-methylpyrrolidone (NMP).
   [I-46] The process according to [I-22], wherein the organic solvent in the reaction in the step ii is acetonitrile.
   [I-47] The process according to any one of [I-6] to [I-14], wherein the amount of the acidic compound used in the step ii is 0.1 mol to 10.0 mol based on 1 mol of the compound of the formula (7).
   [I-48] The process according to any one of [I-6] to [I-14], wherein the amount of the acidic compound used in the step ii is 0.2 mol to 5.0 mol based on 1 mol of the compound of the formula (7).
   [I-49] The process according to any one of [I-6] to [I-14], wherein the amount of the acidic compound used in the step ii is 0.3 mol to 3.0 mol based on 1 mol of the compound of the formula (7).
   [I-50] The process according to any one of [I-6] to [I-14], wherein the amount of the acidic compound (preferably sulfuric acid) used in the step ii is 0.1 mol to 3.0 mol based on 1 mol of the compound of the formula (7).
   [I-51] The process according to any one of [I-6] to [I-14], wherein the amount of the acidic compound (preferably sulfuric acid) used in the step ii is 0.3 mol to 2.0 mol based on 1 mol of the compound of the formula (7).
   [I-52] The process according to any one of [I-6] to [I-14], wherein the amount of the acidic compound (preferably sulfuric acid) used in the step ii is 0.5 mol to 1.0 mol based on 1 mol of the compound of the formula (7).
   [I-53] The process according to any one of [I-6] to [I-14], wherein the amount of the acidic compound (preferably trifluoroacetic acid) used in the step ii is 0.1 mol to 3.0 mol based on 1 mol of the compound of the formula (7).
   [I-54] The process according to any one of [I-6] to [I-14], wherein the amount of the acidic compound (preferably trifluoroacetic acid) used in the step ii is 0.3 mol to 2.0 mol based on 1 mol of the compound of the formula (7).
   [I-55] The process according to any one of [I-6] to [I-14], wherein the amount of the acidic compound (preferably trifluoroacetic acid) used in the step ii is 0.5 mol to 1.0 mol based on 1 mol of the compound of the formula (7).
   [I-56] The process according to any one of [I-1] to [I-55], wherein the reaction in the step ii is performed at 30° C. to 100° C.
   [I-57] The process according to any one of [I-1] to [I-55], wherein the reaction in the step ii is performed at 30° C. to 80° C.
   [I-58] The process according to any one of [I-1] to [I-55], wherein the reaction in the step ii is performed at 40° C. to 80° C.
   [I-59] The process according to any one of [I-22] to [I-46], wherein the amount of the organic solvent used in the reaction in the step ii is 0.3 to 3 liters (preferably 0.3 to 2 liters) based on 1 mol of the compound of the formula (7).
   [I-60] The process according to any one of [I-22] to [I-46], wherein the amount of the organic solvent used in the reaction in the step ii is 0.4 to 1.8 liters based on 1 mol of the compound of the formula (7).
   [I-61] The process according to any one of [I-1] to [I-60], wherein the reaction in the step ii is performed for 1 hour to 48 hours.
   [I-62] The process according to any one of [I-1] to [I-60], wherein the reaction in the step ii is performed for 1 hour to 24 hours.
   [I-63] The process according to [I-22], wherein the organic solvent in the reaction in the step ii is a carboxylic acid.
   [I-64] The process according to [I-22], wherein the organic solvent in the reaction in the step ii is a (C1-C4)alkanoic acid.
   [I-65] The process according to [I-22], wherein the organic solvent in the reaction in the step ii is acetic acid.
   [I-66] The process according to any one of [I-5] to [I-65], wherein the acidic compound in the step ii is selected from sulfuric acid and trifluoroacetic acid.
   [I-67] The process according to any one of [I-5] to [I-65], wherein the amount of the acidic compound (preferably sulfuric acid or trifluoroacetic acid) used in the step ii is 0 (zero) mol to 10.0 mol based on 1 mol of the compound of the formula (7).
   [I-68] The process according to any one of [I-5] to [I-65], wherein the amount of the acidic compound (preferably sulfuric acid or trifluoroacetic acid) used in the step ii is 0 (zero) mol to 5.0 mol based on 1 mol of the compound of the formula (7).
   [I-69] The process according to any one of [I-5] to [I-65], wherein the amount of the acidic compound (preferably sulfuric acid or trifluoroacetic acid) used in the step ii is 0 (zero) mol to 3.0 mol based on 1 mol of the compound of the formula (7).
   [I-70] The process according to any one of [I-1] to [I-69], wherein the reaction in the step ii is performed at 10° C. to 100° C.
   [I-71] The process according to any one of [I-1] to [I-69], wherein the reaction in the step ii is performed at 15° C. to 90° C.
   [I-72] The process according to any one of [I-1] to [I-69], wherein the reaction in the step ii is performed at 20° C. to 80° C.
   [I-73] The process according to any one of [I-22] to [I-46] and [I-63] to [I-65], wherein the amount of the organic solvent used in the reaction in the step ii is 0.3 to 3 liters (preferably 0.3 to 2 liters) based on 1 mol of the compound of the formula (7).
   [I-74] The process according to any one of [I-22] to [I-46] and [I-63] to [I-65], wherein the amount of the organic solvent used in the reaction in the step ii is 0.4 to 1.8 liters based on 1 mol of the compound of the formula (7).
   [I-75] The process according to any one of [I-1] to [I-74], wherein the reaction in the step ii is performed in the presence of a water solvent.
   [I-76] The process according to [I-75], wherein the amount of the water solvent used in the reaction in the step ii is 0.05 to 1.0 liter (preferably 0.1 to 0.5 liters) based on 1 mol of the compound of the formula (7).
   [I-77] The process according to [I-75] or [I-76], wherein the amount of the water solvent in the whole solvent composed of the organic solvent and the water solvent is 5 to 50 vol % (preferably 5 to 40 vol %) based on the amount of the whole solvent.
   [I-78] The process according to any one of [I-1] to [I-77], wherein the reaction in the step ii is performed for 1 hour to 48 hours.
   [I-79] The process according to any one of [I-1] to [I-77], wherein the reaction in the step ii is performed for 1 hour to 24 hours.
   [I-80] The process according to any one of [I-1] to [I-4], wherein the reaction in the step ii is performed in the presence of a base.
   [I-81] The process according to [I-80], wherein the base in the step ii is selected from metal hydrogen carbonates and metal carbonates.
   [I-82] The process according to [I-80], wherein the base in the step ii is selected from alkali metal hydrogen carbonates, alkali metal carbonates, alkaline earth metal hydrogen carbonates, and alkaline earth metal carbonates.
   [I-83] The process according to [I-80], wherein the base in the step ii is selected from alkali metal hydrogen carbonates and alkali metal carbonates.
   [I-84] The process according to [I-80], wherein the base in the step ii is an alkali metal carbonate, an alkali metal hydrogen carbonate, or a mixture thereof.
   [I-85] The process according to [I-80], wherein the base in the step ii is selected from lithium hydrogen carbonate, sodium hydrogen carbonate, potassium hydrogen carbonate, cesium hydrogen carbonate, magnesium hydrogen carbonate, calcium hydrogen carbonate, lithium carbonate, sodium carbonate, potassium carbonate, cesium carbonate, magnesium carbonate, and calcium carbonate.
   [I-86] The process according to [I-80], wherein the base in the step ii is lithium hydrogen carbonate, sodium hydrogen carbonate, potassium hydrogen carbonate, cesium hydrogen carbonate, magnesium hydrogen carbonate, calcium hydrogen carbonate, lithium carbonate, sodium carbonate, potassium carbonate, cesium carbonate, magnesium carbonate, calcium carbonate, or a mixture thereof.
   [I-87] The process according to [I-80], wherein the base in the step ii is selected from sodium hydrogen carbonate, potassium hydrogen carbonate, sodium carbonate, and potassium carbonate.
   [I-88] The process according to [I-80], wherein the base in the step ii is sodium hydrogen carbonate, potassium hydrogen carbonate, sodium carbonate, potassium carbonate, or a mixture thereof.
   [I-89] The process according to [I-80], wherein the base in the step ii is selected from sodium carbonate, and potassium carbonate.
   [I-90] The process according to [I-80], wherein the base in the step ii is sodium carbonate, or potassium carbonate.
   [I-91] The process according to [I-80], wherein the base in the step ii is sodium carbonate.
   [I-92] The process according to [I-80], wherein the base in the step ii is potassium carbonate.
   [I-93] The process according to any one of [I-80] to [I-92], wherein the amount of the base used in the step ii is 0.01 to 1 mol based on 1 mol of the compound of the formula (7).
   [I-94] The process according to any one of [I-80] to [I-92], wherein the amount of the base used in the step ii is 0.05 to 1 mol based on 1 mol of the compound of the formula (7).
   [I-95] The process according to any one of [I-80] to [I-92], wherein the amount of the base used in the step ii is 0.1 to 0.8 mol based on 1 mol of the compound of the formula (7).
   [I-96] The process according to any one of [I-80] to [I-92], wherein the amount of the base used in the step ii is 0.05 to 5 mol (preferably 0.1 to 3 mol) based on 1 mol of the compound of the formula (7).
   [I-97] The process according to any one of [I-80] to [I-92], wherein the amount of the base used in the step ii is 0.4 to 1.5 based on 1 mol of the compound of the formula (7).
   [I-98] The process according to any one of [I-80] to [I-92], wherein the amount of the base used in the step ii is 0.2 to 2 mol based on 1 mol of the compound of the formula (7).
   [I-99] The process according to any one of [I-80] to [I-92], comprising simultaneously adding the base in the step ii and the oxidizing agent in the step ii.
   [I-100] The process according to any one of [I-80] to [I-92], wherein the base in the step ii and the oxidizing agent in the step ii are simultaneously added.
   [I-101] The process according to any one of [I-80] to [I-92], wherein the addition rate of the base in the step ii is 0.03 mol/hr. to 0.5 mol/hr. based on 1 mol of the compound of the formula (7).
   [I-102] The process according to any one of [I-80] to [I-92], wherein the addition rate of the hydrogen peroxide in the step ii is 0.13 mol/hr. to 1.0 mol/hr. based on 1 mol of the compound of the formula (7).
   [I-103] The process according to any one of [I-80] to [I-92], wherein the addition rate of the oxidizing agent in the step ii is 1 time to 30 times (preferably over 1 time and 30 times or less) the addition rate of the base in the step ii.
   [I-104] The process according to any one of [I-80] to [I-92], wherein the addition rate of the oxidizing agent in the step ii is 1 time to 20 times (preferably over 1 time and 20 times or less) the addition rate of the base in the step ii.
   [I-105] The process according to any one of [I-80] to [I-92], wherein the addition rate of the oxidizing agent in the step ii is 1 time to 10 times (preferably over 1 time and 10 times or less) the addition rate of the base in the step ii.
   [I-106] The process according to any one of [I-80] to [I-92], wherein the addition rate of the base in the step ii is the same as the addition rate of the oxidizing agent in the step ii.
   [I-107] The process according to any one of [I-80] to [I-92], wherein the addition rate of the oxidizing agent in the step ii is higher than the addition rate of the base in the step ii.
   [I-108] The process according to any one of [I-80] to [I-107], wherein the addition time of the base in the step ii is 1 hour to 48 hours.
   [I-109] The process according to any one of [I-80] to [I-107], wherein the addition time of the base in the step ii is 1 hour to 24 hours.
   [I-110] The process according to any one of [I-80] to [I-107], wherein the addition time of the oxidizing agent in the step ii is 1 hour to 48 hours.
   [I-111] The process according to any one of [I-80] to [I-107], wherein the addition time of the oxidizing agent in the step ii is 1 hour to 24 hours.
   [I-112] The process according to any one of [I-80] to [I-107], wherein the aging time after adding the base and the oxidizing agent in the step ii is 0.1 hours to 12 hours.
   [I-113] The process according to any one of [I-80] to [I-107], wherein the aging time after adding the base and the oxidizing agent in the step ii is 0.2 hours to 9 hours.
   [I-114] The process according to any one of [I-80] to [I-107], wherein the aging time after adding the base and the oxidizing agent in the step ii is 0.5 hours to 6 hours.
   [I-115] The process according to any one of [I-80] to [I-114], wherein the reaction in the step ii is performed in the presence of a nitrile compound.
   [I-116] The process according to [I-115], wherein the nitrile compound in the step ii is an alkylnitrile derivative, a benzonitrile derivative, or a mixture thereof.
   [I-117] The process according to [I-115], wherein the nitrile compound in the step ii is acetonitrile, propionitrile, butyronitrile, isobutyronitrile, succinonitrile, benzonitrile, p-nitrobenzonitrile, or a mixture thereof.
   [I-118] The process according to [I-115], wherein the nitrile compound in the step ii is acetonitrile, isobutyronitrile, succinonitrile, benzonitrile, p-nitrobenzonitrile, or a mixture thereof.
   [I-119] The process according to [I-115], wherein the nitrile compound in the step ii is acetonitrile.
   [I-120] The process according to any one of [I-115] to [I-119], wherein the amount of the nitrile compound used in the step ii is 1 to 100 mol (preferably 1 to 50 mol) based on 1 mole of the compound of the formula (7).
   [I-121] The process according to any one of [I-115] to [I-119], wherein the amount of the nitrile compound used in the step ii is 1 to 35 mol based on 1 mole of the compound of the formula (7).
   [I-122] The process according to any one of [I-80] to [I-121], wherein the reaction in the step ii is performed in the presence of a ketone compound.
   [I-123] The process according to [I-122], wherein the ketone compound in the step ii is 2,2,2-trifluoroacetophenone.
   [I-124] The process according to [I-122] or [I-123], wherein the amount of the ketone compound used in the step ii is 0.01 to 1.0 mol based on 1 mol of the compound of the formula (7).
   [I-125] The process according to [I-122] or [I-123], wherein the amount of the ketone compound used in the step ii is 0.05 to 0.8 mol based on 1 mol of the compound of the formula (7).
   [I-126] The process according to [I-122] or [I-123], wherein the amount of the ketone compound used in the step ii is 0.1 to 0.6 mol based on 1 mol of the compound of the formula (7).
   [I-127] The process according to any one of [I-80] to [I-126], wherein the reaction in the step ii is performed in the presence of an organic solvent.
   [I-128] The process according to [I-127], wherein the organic solvent in the reaction in the step ii is selected from aromatic hydrocarbon derivatives, halogenated aliphatic hydrocarbons, alcohols, nitriles, carboxylic acid esters, ethers, ketones, amides, and ureas.
   [I-129] The process according to [I-127], wherein the organic solvent in the reaction in the step ii is one or more (preferably one or two, and more preferable one) organic solvents selected from aromatic hydrocarbon derivatives, halogenated aliphatic hydrocarbons, alcohols, nitriles, carboxylic acid esters, ethers, ketones, amides, and ureas.
   [I-130] The process according to [I-127], wherein the organic solvent in the reaction in the step ii is one or more (preferably one or two, and more preferably one) organic solvents selected from alcohols, nitriles, carboxylic acid esters, and amides.
   [I-131] The process according to [I-127], [I-54], or [I-50], wherein the organic solvent in the reaction in the step ii is one or more (preferably one or two, and more preferably one) organic solvents selected from alcohols, nitriles, and carboxylic acid esters.
   [I-132] The process according to [I-127], wherein the organic solvent in the reaction in the step ii is selected from alcohols, nitriles, and amides.
   [I-133] The process according to [I-127], wherein the organic solvent in the reaction in the step ii is one or more (preferably one or two, and more preferably one) organic solvents selected from alcohols, nitriles, and amides.
   [I-134] The process according to [I-127], wherein the organic solvent in the reaction in the step ii is one or more (preferably one or two, and more preferable one) organic solvents selected from the group consisting of methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, propionitrile, butyronitrile, isobutyronitrile, succinonitrile, benzonitrile, N,N-dimethylformamide, and N,N-dimethylacetamide.
   [I-135] The process according to [I-127], wherein the organic solvent in the reaction in the step ii is one or more (preferably one or two, and more preferable one) organic solvents selected from nitriles and amides.
   [I-136] The process according to [I-127], wherein the organic solvent in the reaction in the step ii is one or more (preferably one or two, and more preferable one) organic solvents selected from the group consisting of acetonitrile, propionitrile, butyronitrile, isobutyronitrile, succinonitrile, benzonitrile, N,N-dimethylformamide, and N,N-dimethylacetamide.
   [I-137] The process according to [I-127], wherein the organic solvent in the reaction in the step ii is a nitrile.
   [I-138] The process according to [I-127], wherein the organic solvent in the reaction in the step ii is selected from the group consisting of acetonitrile, propionitrile, butyronitrile, isobutyronitrile, succinonitrile, and benzonitrile.
   [I-139] The process according to [I-127], wherein the organic solvent in the reaction in the step ii is one or more organic solvents selected from the group consisting of acetonitrile, propionitrile, butyronitrile, isobutyronitrile, succinonitrile, and benzonitrile.
   [I-140] The process according to [I-127], wherein the organic solvent in the reaction in the step ii is one or two organic solvents selected from the group consisting of acetonitrile, propionitrile, butyronitrile, isobutyronitrile, succinonitrile, and benzonitrile.
   [I-141] The process according to [I-127], wherein the organic solvent in the reaction in the step ii is one organic solvent selected from the group consisting of acetonitrile, propionitrile, butyronitrile, isobutyronitrile, succinonitrile, and benzonitrile.
   [I-142] The process according to [I-127], wherein the organic solvent in the reaction in the step ii is acetonitrile.
   [I-143] The process according to any one of [I-127] to [I-142], wherein the amount of the organic solvent used in the reaction in the step ii is 0.5 to 3 liters (preferably 1 to 3 liters) based on 1 mol of the compound of the formula (7).
   [I-144] The process according to any one of [I-127] to [I-142], wherein the amount of the organic solvent used in the reaction in the step ii is 1 to 2 liters based on 1 mol of the compound of the formula (7).
   [I-145] The process according to [I-80] to [I-144], wherein the reaction in the step ii is performed in the presence of a water solvent.
   [I-146] The process according to [I-145], wherein the amount of the water solvent used in the reaction in the step ii is 0.5 to 2.0 liters (preferably 0.8 to 1.5 liters) based on 1 mol of the compound of the formula (7).
   [I-147] The process according to [I-145] to [I-146], wherein the amount of the water solvent in the whole solvent composed of the organic solvent and the water solvent is 20 to 60 vol % (preferably 30 to 50 vol %) based on the amount of the whole solvent.
   [I-148] The process according to any one of [I-80] to [I-147], wherein the reaction in the step ii is performed at 0° C. to 80° C.
   [I-149] The process according to any one of [I-80] to [I-147], wherein the reaction in the step ii is performed at 5° C. to 60° C. (preferably 10° C. to 40° C.).
   [I-150] The process according to any one of [I-80] to [I-147], wherein the reaction in the step ii is performed for 5 minutes to 48 hours (preferably 10 minutes to 24 hours).
   [I-151] The process according to any one of [I-1] to [I-4], wherein the compound of the formula (7) is reacted with the oxidizing agent under acidic conditions, and the resultant is then reacted with the oxidizing agent under neutral to alkaline conditions in the reaction in the step ii.
   [I-152] The process according to any one of [I-1] to [I-4], wherein the reaction in the step ii includes the process according to any one of [I-5] to [I-79] and the process according to any one of [I-80] to [I-150].
   [I-153] The process according to any one of [I-1] to [I-152], wherein the oxidizing agent in the step ii is hydrogen peroxide, a persulfate, or a hydrogen persulfate.
   [I-154] The process according to any one of [I-1] to [I-152], wherein the oxidizing agent in the step ii is hydrogen peroxide.
   [I-155] The process according to any one of [I-1] to [I-152], wherein the oxidizing agent in the step ii is an alkali metal persulfate, an ammonium persulfate salt, or an alkali metal hydrogen persulfate.
   [I-156] The process according to any one of [I-1] to [I-152], wherein the oxidizing agent in the step ii is sodium hydrogen persulfate, potassium hydrogen persulfate, potassium persulfate, sodium persulfate, or ammonium persulfate.
   [I-157] The process according to any one of [I-1] to [I-152], wherein the oxidizing agent in the step ii is potassium hydrogen persulfate.
   [I-158] The process according to [I-153] to [I-157], wherein the organic solvent in the reaction in the step ii is a nitrile or an amide (preferably acetonitrile or N,N-dimethylformamide).
   [I-159] The process according to [I-153] to [I-157], wherein the organic solvent in the reaction in the step ii is a nitrile.
   [I-160] The process according to [I-153] to [I-157], wherein the organic solvent in the reaction in the step ii is acetonitrile.
   [I-161] The process according to any one of [I-153] to [I-160], wherein the amount of the organic solvent used in the reaction in the step ii is 0.3 to 1.3 liters (preferably 0.7 to 1.0 liter) based on 1 mol of the compound of the formula (7).
   [I-162] The process according to [I-153] to [I-161], wherein the reaction in the step ii is performed in the presence of a water solvent.
   [I-163] The process according to [I-162], wherein the amount of the water solvent used in the reaction in the step ii is 1.0 to 4.0 liters (preferably 2.0 to 3.0 liters) based on 1 mol of the compound of the formula (7).
   [I-164] The process according to [I-162] or [I-163], wherein the amount of the water solvent in the whole solvent composed of the organic solvent and the water solvent is 65 to 85 vol % (preferably 70 to 80 vol %) based on the amount of the whole solvent.
   [I-165] The process according to any one of [I-153] to [I-164], wherein the reaction in the step ii is performed at 20° C. to 100° C.
   [I-166] The process according to any one of [I-153] to [I-164], wherein the reaction in the step ii is performed at 30° C. to 90° C.
   [I-167] The process according to any one of [I-153] to [I-166], wherein the reaction in the step ii is performed for 1 hour to 48 hours.
   [I-168] The process according to any one of [I-153] to [I-166], wherein the reaction in the step ii is performed for 1 hour to 24 hours.
   [I-169] The process according to any one of [I-1] to [I-168], wherein the oxidizing agent in the step ii is a 10 to 70 wt % aqueous hydrogen peroxide solution, with the proviso that any process not using hydrogen peroxide as the oxidizing agent is excluded.
   [I-170] The process according to any one of [I-1] to [I-168], wherein the oxidizing agent in the step ii is a 25 to 65 wt % aqueous hydrogen peroxide solution, with the proviso that any process not using hydrogen peroxide as the oxidizing agent is excluded.
   [I-171] The process according to any one of [I-1] to [I-170], wherein the amount of the oxidizing agent used in the step ii is 2 to 8 mol (preferably 2 to 6 mol) based on 1 mol of the compound of the formula (7).
   [I-172] The process according to any one of [I-1] to [I-170], wherein the amount of the oxidizing agent used in the step ii is 2 to 5 mol (preferably 2 to 4 mol) based on 1 mol of the compound of the formula (7).
   [I-173] The process according to any one of [I-1] to [I-170], wherein the amount of the oxidizing agent used in the step ii is 3 to 6 mol based on 1 mol of the compound of the formula (7).
   [I-174] The process according to any one of [I-1] to [I-170], wherein the amount of the oxidizing agent used in the step ii is 1.0 to 2.0 mol (preferably 1.0 to 1.5 mol) based on 1 mol of the compound of the formula (7).
   [I-175] The process according to any one of [I-1] to [I-170], wherein the amount of the oxidizing agent used in the step ii is 1.0 to 1.5 mol based on 1 mol of the compound of the formula (7).
   [I-176] The process according to any one of [I-1] to [I-175], wherein the process excludes an acidic compound not immobilized.
   [I-178] The process according to any one of [I-1] to [I-175], wherein the process excludes a base not immobilized.
   [I-176] The process according to any one of [I-1] to [I-175], wherein
in the formulas (7) and (8),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.
   [I-177] The process according to any one of [I-1] to [I-175], wherein
in the formulas (7) and (8),
R¹ is methyl,
R² is trifluoromethyl,
R³ is difluoromethyl, and
R⁴ and R⁵ are methyl.

In another aspect, the present invention is as follows.

[II-1] The process according to any one of [I-1] to [I-175] comprising, before the step ii, the following step i-a:
(step i-a) reacting a compound of the formula (1) with a compound of the formula (2) in the presence of a base to produce the compound of the formula (7):

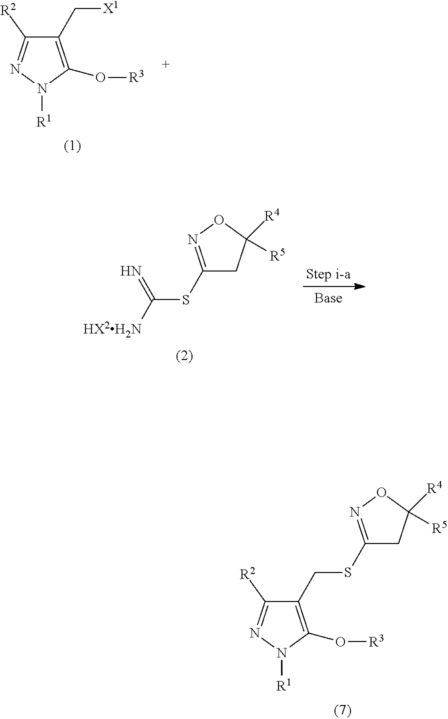

wherein in the formula (1), R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and X¹ is a leaving group, and
in the formula (2), R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents,
X² is an atom or an atomic group forming an acid, and
in the formula (7), R¹, R², R³, R⁴, and R⁵ are as defined above.
   [II-2] The process according to any one of [I-1] to [I-175], comprising, before the step ii, the following step 1-b:
(step i-b) reacting a compound of the formula (4) with a compound of the formula (3) in the presence of a base to produce the compound of the formula (7):

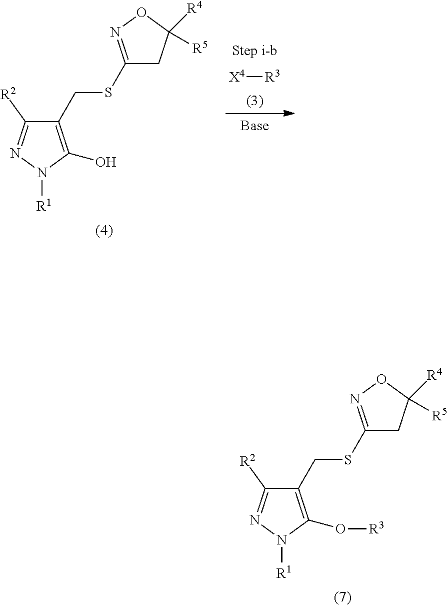

wherein in the formula (3), the formula (4), and the formula (7), R¹, R², R³, R⁴, and R⁵ are as defined above, and
X⁴ is a leaving group.
   [II-3] The process according to any one of [I-1] to [I-175], comprising, before the step ii, the following step i-C:
(step i-c) reacting a compound of the formula (5) with a compound of the formula (6) in the presence of a base to produce the compound of the formula (7):

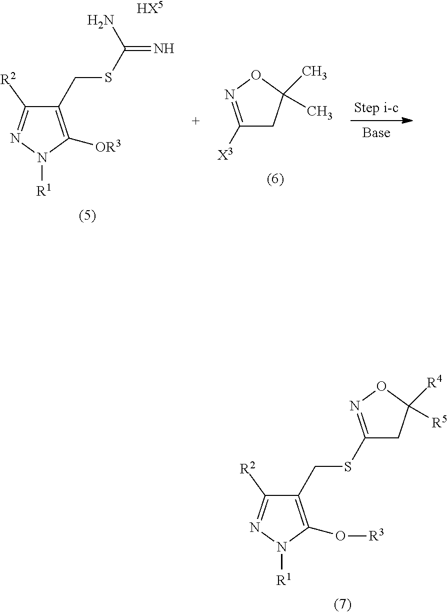

wherein in the formula (5), the formula (6), and the formula (7), R¹, R², R³, R⁴, and R⁵ are as defined above, XV is a leaving group, and X⁵ is an atom or an atomic group forming an acid.
   [II-4] The process according to any one of [II-1] to [II-3], wherein the base in the step i-a, i-b, or i-c is an alkali metal hydroxide, an alkali metal carbonate, or a mixture thereof.
   [II-5] The process according to any one of [II-1] to [II-3], wherein the base in the step i-a, i-b, or i-c is sodium hydroxide, potassium hydroxide, sodium carbonate, potassium carbonate, or a mixture thereof.
   [II-6] The process according to any one of [II-1] to [II-3], wherein the base in the step i-a, i-b, or i-c is an alkali metal hydroxide.
   [II-7] The process according to any one of [II-1] to [II-3], wherein the base in the step i-a, i-b, or i-c is sodium hydroxide or potassium hydroxide.
   [II-8] The process according to any one of [II-1] to [II-3], wherein the base in the step i-a, i-b, or i-c is an alkali metal carbonate.
   [II-9] The process according to any one of [II-1] to [II-3], wherein the base in the step i-a, i-b, or i-c is potassium carbonate, or sodium carbonate.
   [II-10] The process according to any one of [II-1] to [II-9], wherein the reaction in the step i-a, i-b, or i-c is performed in the presence of a solvent.
   [II-11] The process according to [II-10], wherein the organic solvent in the reaction in the step i-a, i-b, or i-c is an aromatic hydrocarbon derivative, a halogenated aliphatic hydrocarbon, an alcohol, a nitrile, a carboxylic acid ester, an ether, a ketone, an amide, a urea, a sulfoxide, a sulfone, water or a mixture thereof.
   [II-12] The process according to [II-10], wherein the organic solvent in the step i-a, i-b, or i-c is an alcohol, a nitrile, a carboxylic acid ester, an ether, an amide, a sulfone, water or a mixture thereof.
   [II-13] The process according to any one of [II-10] to [II-12], wherein the amount of the solvent used in the reaction in the step i-a, i-b, or i-c is 1 to 3 liters based on 1 mol of the compound of the formula (1), the formula (4), or the formula (5) corresponding to the reaction.
   [II-14] The process according to any one of [II-10] to [II-12], wherein the total amount of the solvent used in the reaction in the step i-a, i-b, or i-c is 1.5 to 3.0 liters based on 1 mol of the compound of the formula (1), the formula (4), or the formula (5) corresponding to the reaction.
   [II-15] The process according to any one of [II-10] to [II-12], wherein the total amount of the solvent used in the reaction in the step i-a, i-b, or i-c is 1.5 to 2.5 liters based on 1 mol of the compound of the formula (1), the formula (4), or the formula (5) corresponding to the reaction.
   [II-16] The process according to any one of [II-10] to [II-12], wherein the total amount of the solvent used in the reaction in the step i-a, i-b, or i-c is 1.7 to 2.0 liters based on 1 mol of the compound of the formula (1), the formula (4), or the formula (5) corresponding to the reaction.
   [II-17] The process according to any one of [II-1] to [II-16], wherein the reaction in the step i-a, i-b, or i-c is performed at −10° C. to 100° C.
   [II-18] The process according to any one of [II-1] to [II-16], wherein the reaction in the step i-a, i-b, or i-c is performed at −10° C. to 70° C.
   [II-19] The process according to any one of [II-1] to [II-16], wherein the reaction in the step i-a, i-b, or i-c is performed at −10° C. to 50° C.
   [II-20] The process according to any one of [II-1] to [II-16], wherein the reaction in the step i-a, i-b, or i-c is performed at 0° C. to 40° C.
   [II-21] The process according to any one of [II-1] to [II-16], wherein the reaction in the step i-a, i-b, or i-c is performed at 0° C. to 30° C.
   [II-22] The process according to any one of [II-1] to [II-21], wherein the reaction in the step i-a, i-b, or i-c is performed for 1 hour to 48 hours.
   [II-23] The process according to any one of [II-1] to [II-21], wherein the reaction in the step i-a, i-b, or i-c is performed for 1 hour to 24 hours.
   [II-24] The process according to any one of [II-1] to [II-21], wherein the reaction in the step i-a, i-b, or i-c is performed for 4 hours to 24 hours.
   [II-25] The process according to [II-1], wherein in the formula (1),
R¹ is a (C1-C4)alkyl,
R² is a (C1-C4)perfluoroalkyl,
R³ is a (C1-C4)alkyl optionally substituted with 1 to 9 fluorine atoms,
X¹ is a chlorine atom or a bromine atom, and
in the formula (2),
R⁴ and R⁵ are each independently a (C1-C4)alkyl,
X² is a chlorine atom, a bromine atom, a sulfate group, a hydrogen sulfate group, a phosphate group, a monohydrogen phosphate group, methanesulfonyloxy, p-toluenesulfonyloxy, or a mixture of two or more thereof, and in the formula (7), R¹, R², R³, R⁴ and R⁵ are as defined above.
   [II-26] The process according to [II-1], wherein in the formula (1),
R¹ is methyl,
R² is trifluoromethyl,
R³ is difluoromethyl,
X¹ is a chlorine atom,
in the formula (2),
R⁴ and R⁵ are methyl,
X² is a chlorine atom, a bromine atom, or a mixture thereof,
in the formula (7) and the formula (8), R¹, R², R³, R⁴ and R⁵ are as defined above.
   [II-27] The process according to [II-2], wherein in the formula (3),
R³ is a (C1-C4)alkyl optionally substituted with 1 to 9 fluorine atoms,
X⁴ is a chlorine atom or a bromine atom,
in the formula (4),
R¹ is a (C1-C4)alkyl,
R² is a (C1-C4)perfluoroalkyl,
R⁴ and R⁵ are each independently a (C1-C4)alkyl, and
in the formula (7) and the formula (8), R¹, R², R³, R⁴ and R⁵ are as defined above.
   [II-28] The process according to [II-2], wherein in the formula (3),
R³ is difluoromethyl,
X⁴ is a chlorine atom or a bromine atom,
in the formula (4),
R¹ is methyl,
R² is trifluoromethyl,
R⁴ and R⁵ are methyl, and
in the formula (7) and the formula (8), R¹, R², R³, R⁴ and R⁵ are as defined above.
   [II-29] The process according to [II-3], wherein in the formula (5),
R¹ is a (C1-C4)alkyl,
R² is a (C1-C4)perfluoroalkyl,
R³ is a (C1-C4)alkyl optionally substituted with 1 to 9 fluorine atoms,
X⁵ is a chlorine atom, a bromine atom, or a mixture thereof,
in the formula (6),
R⁴ and R⁵ are each independently a (C1-C4)alkyl,
X³ is a chlorine atom or a bromine atom, and
in the formula (7) and the formula (8), R¹, R², R³, R⁴ and R⁵ are as defined above.
   [II-30] The process according to [II-3], wherein in the formula (5),
R¹ is methyl,
R² is trifluoromethyl,
R³ is difluoromethyl,
X⁵ is a chlorine atom, a bromine atom, or a mixture thereof,
in the formula (6),
R⁴ and R⁵ are methyl,
X³ is a chlorine atom or a bromine atom, and
in the formula (7) and the formula (8), R¹, R², R³, R⁴ and R⁵, are as defined above.
   [III-1] A process for producing a compound of the formula (8), the process comprising the following step ii:
(step ii) reacting a compound of the formula (7) with an oxidizing agent in the absence of a transition metal and in the presence of a base to produce the compound of the formula (8):

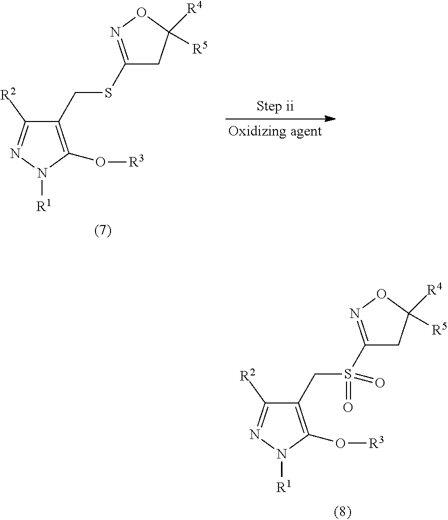

wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents.
   [III-2] The process according to [III-1], wherein the reaction in the step ii is performed in the presence of an organic solvent, and the organic solvent is an organic solvent other than an alcohol.
   [III-3] The process according to [III-1] or [III-2], wherein the organic solvent is acetonitrile.
   [III-4] The process according to any one of [III-1] to [III-3], comprising simultaneously adding the base in the step ii and the oxidizing agent in the step ii.
   [III-5] The process according to any one of [III-1] to [III-4], wherein the base in the step ii is selected from sodium hydrogen carbonate, potassium hydrogen carbonate, sodium carbonate and potassium carbonate.
   [III-6] The process according to any one of [III-1] to [III-5], wherein the oxidizing agent in the step ii is hydrogen peroxide.
   [III-7] A process for producing a compound of the formula (8), the process comprising the following step ii:
(step ii) reacting a compound of the formula (7) with an oxidizing agent in the absence of a transition metal and in the presence of an acidic compound to produce the compound of the formula (8), wherein the acidic compound is sulfuric acid:

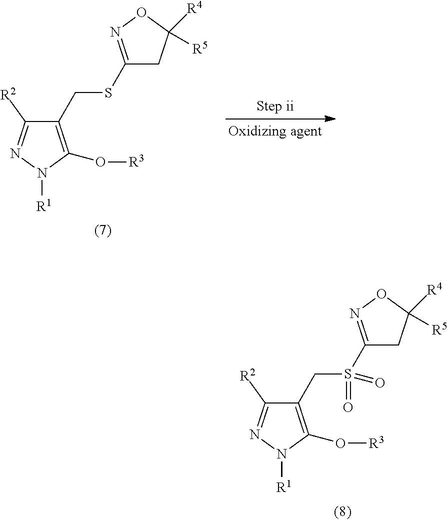

wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents.
   [III-8] The process according to [III-7], wherein the reaction in the step ii is performed in the presence of an organic solvent having an acceptor number of 5 to 25 and a relative permittivity of 1 to 40.
   [III-9] The process according to [III-7], wherein the reaction in the step ii is performed in the presence of an organic solvent having an acceptor number of 5 to 25 and a Rohrschneider's polarity parameter of 1 to 7.
   [III-10] The process according to any one of [III-7] to [III-9], wherein the organic solvent is an organic solvent other than an alcohol.
   [III-11] The process according to any one of [III-7] to [III-10], wherein the organic solvent is selected from aromatic hydrocarbon derivatives, nitriles, carboxylic acid esters, and amides.
   [III-12] The process according to any one of [III-7] to [III-11], wherein the oxidizing agent in the step ii is hydrogen peroxide.
   [III-13] A process for producing a compound of the formula (8), the process comprising the following step ii:
(step ii) reacting a compound of the formula (7) with an oxidizing agent in the absence of a transition metal and in the presence of an acidic compound to produce the compound of the formula (8), wherein the acidic compound is a (C2-C4)alkanoic acid substituted with 1 to 7 fluorine atoms:

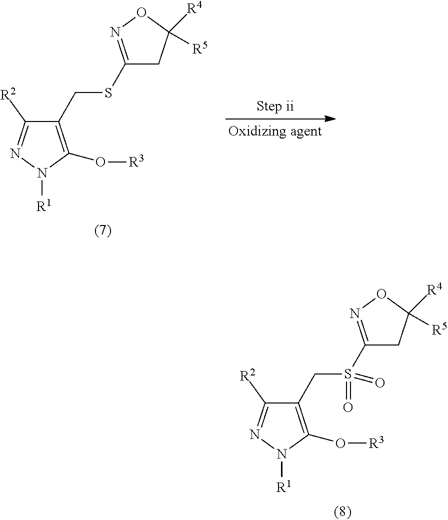

wherein in the formula (7) and the formula (8), R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents.
   [III-14] The process according to [III-13], wherein the (C2-C4)alkanoic acid substituted with 1 to 7 fluorine atoms is trifluoroacetic acid.
   [III-15] The process according to [III-13] or [III-14], wherein the oxidizing agent in the step ii is hydrogen peroxide.
   [III-16] A process for producing a compound of the formula (8), the process comprising the following step ii:
(step ii) reacting a compound of the formula (7) with an oxidizing agent in the absence of a transition metal and in the presence of an organic solvent to produce the compound of the formula (8), wherein the organic solvent is a (C1-C4)alkanoic acid:

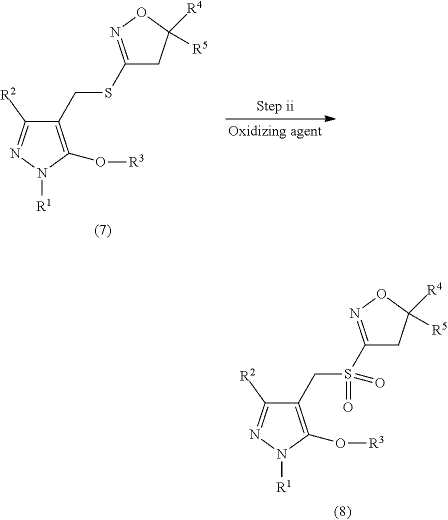

wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents.
   [III-17] The process according to [III-16], wherein the (C1-C4)alkanoic acid is acetic acid.
   [III-18] The process according to [III-16] or [III-17], wherein the oxidizing agent in the step ii is hydrogen peroxide.
   [III-19] A process for producing a compound of the formula (8), the process comprising the following step ii:
(step ii) reacting a compound of the formula (7) with an oxidizing agent in the absence of a transition metal to produce the compound of the formula (8), wherein the oxidizing agent is an alkali metal persulfate, an ammonium persulfate salt, or an alkali metal hydrogen persulfate:

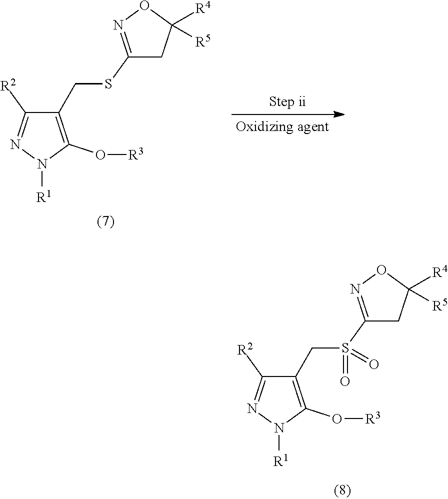

wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with the carbon atom to which they are attached, form a 4- to 12-membered carbocyclic ring, wherein the carbocyclic ring is optionally substituted with one or more substituents.
   [III-20] The process according to [III-19], wherein the oxidizing agent is sodium hydrogen persulfate, potassium hydrogen persulfate, potassium persulfate, sodium persulfate, or ammonium persulfate.
   [III-21] The process according to [III-20], wherein the reaction in the step ii is performed in the presence of an organic solvent, and the organic solvent is acetonitrile.
   [III-22] The process according to any one of [III-1] to [III-21],
wherein in the formula (7) and the formula (8),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.
   [III-23] The process according to any one of [III-1] to [III-21],
wherein in the formula (7) and the formula (8),
R¹ is methyl,
R² is trifluoromethyl,
R³ is difluoromethyl, and
R⁴ and R⁵ are methyl.

The symbols and terms described in the present description will be explained.

Herein, the following abbreviations and prefixes may be used, and their meanings are as follows:
Me: methyl
Et: ethyl
Pr, n-Pr and Pr-n: propyl (i.e., normal propyl)
i-Pr and Pr-i: isopropyl
Bu, n-Bu and Bu-n: butyl (i.e., normal butyl)
s-Bu and Bu-s: sec-butyl (i.e., secondary butyl)
i-Bu and Bu-i: isobutyl
t-Bu and Bu-t: tert-butyl (i.e., tertiary butyl)
Ph: phenyl
n-: normal
s- and sec-: secondary
i- and iso-: iso
t- and tert-: tertiary
c- and cyc-: cyclo
o-: ortho
m-: meta
p-: para

The term “nitro” means the substituent “—NO₂”.

The term “cyano” or “nitrile” means the substituent “—CN”.

The term “hydroxy” means the substituent “—OH”.

The term “amino” means the substituent “—NH₂”.

(Ca-Cb) means that the number of carbon atoms is a to b. For example, “(C1-C4)” in “(C1-C4)alkyl” means that the number of the carbon atoms in the alkyl is 1 to 4, and “(C2-C5)” means that the number of the carbon atoms in the alkyl is 2 to 5. “(Ca-Cb)” meaning the number of carbon atoms may be written as “Ca-Cb” without parentheses. Thus, for example, “C1-C4” in “C1-C4 alkyl” means that the number of the carbon atoms in the alkyl is 1 to 4.

Herein, it is to be interpreted that generic terms such as “alkyl” include both the straight chain and the branched chain such as butyl and tert-butyl. Meanwhile, for example, the specific term “butyl” refers to straight “normal butyl”, and does not refer to branched “tert-butyl”. Branched chain isomers such as “tert-butyl” are referred to specifically when intended.

Examples of the halogen atom include fluorine atom, chlorine atom, bromine atom and iodine.

The (C1-C6)alkyl means a straight or branched alkyl having 1 to 6 carbon atoms. Examples of the (C1-C6)alkyl include, but are not limited to, methyl, ethyl, propyl, isopropyl, butyl, sec-butyl, isobutyl, tert-butyl, pentyl and hexyl.

The (C1-C4)alkyl means a straight or branched alkyl having 1 to 4 carbon atoms. Examples of the (C1-C4)alkyl include, appropriate examples of the examples of the (C1-C6)alkyl above-mentioned.

The (C3-C6)cycloalkyl means a cycloalkyl having 3 to 6 carbon atoms. Examples of the (C3-C6)cycloalkyl are cyclopropyl, cyclobutyl, cyclopentyl and cyclohexyl.

The (C2-C6)alkenyl means a straight or branched alkenyl having 2 to 6 carbon atoms. Examples of the (C2-C6)alkenyl include, but are not limited to, vinyl, 1-propenyl, isopropenyl, 2-propenyl, 1-butenyl, 1-methyl-1-propenyl, 2-methyl-1-propenyl, 2-butenyl, 3-butenyl, 1,3-butadienyl, 1-pentenyl and 1-hexenyl.

The (C2-C6)alkynyl means a straight or branched alkynyl having 2 to 6 carbon atoms. Examples of the (C2-C6)alkynyl include, but are not limited to, ethynyl, 1-propynyl, 2-propynyl, 1-butynyl, 1-methyl-2-propynyl, 2-butynyl, 3-butynyl, 1-pentynyl and 1-hexynyl.

Examples of the (C6-C10)aryl are phenyl, 1-naphthyl and 2-naphthyl.

The (C1-C6)haloalkyl means a straight or branched alkyl having 1 to 6 carbon atoms which is substituted with 1 to 13 same or different halogen atoms, wherein the halogen atoms have the same meaning as defined above. Examples of the (C1-C6)haloalkyl include, but are not limited to, fluoromethyl, chloromethyl, bromomethyl, difluoromethyl, dichloromethyl, trifluoromethyl, trichloromethyl, chlorodifluoromethyl, bromodifluoromethyl, 2-fluoroethyl, 1-chloroethyl, 2-chloroethyl, 2,2,2-trifluoroethyl, pentafluoroethyl, 3-fluoropropyl, 3-chloropropyl, 2-chloro-1-methylethyl, 2,2,3,3,3-pentafluoropropyl, 2,2,2-trifluoro-1-trifluoromethylethyl, heptafluoropropyl, 1,2,2,2-tetrafluoro-1-trifluoromethylethyl, 4-fluorobutyl, 4-chlorobutyl, 2,2,3,3,4,4,4-heptafluorobutyl, nonafluorobutyl, 1,1,2,3,3,3-hexafluoro-2-trifluoromethylpropyl, 2,2,2-trifluoro-1,1-di(trifluoromethyl)ethyl, undecafluoropentyl and tridecafluorohexyl.

The (C1-C4)perfluoroalkyl means a straight or branched alkyl having 1 to 4 carbon atoms, wherein all hydrogen atoms are substituted with fluorine atoms. Examples of the (C1-C4)perfluoroalkyl are trifluoromethyl (i.e., —CF₃), pentafluoroethyl (i.e., —CF₂CF₃), heptafluoropropyl (i.e., —CF₂CF₂CF₃), 1,2,2,2-tetrafluoro-1-trifluoromethylethyl (i.e., —CF(CF₃)₂), nonafluorobutyl, (i.e., —CF₂CF₂CF₂CF₃), 1,2,2,3,3,3-hexafluoro-1-trifluoromethylpropyl (i.e., —CF(CF₃)CF₂CF₃), 1,1,2,3,3,3-hexafluoro-2-trifluoromethylpropyl (i.e., —CF₂CF(CF₃)₂) and 2,2,2-trifluoro-1,1-di(trifluoromethyl) ethyl (i.e., —C(CF₃)₃).

The (C1-C6)alkoxy means a (C1-C6)alkyl-O—, wherein the (C1-C6)alkyl moiety has the same meaning as defined above. Examples of the (C1-C6)alkoxy include, but are not limited to, methoxy, ethoxy, propoxy, isopropoxy, butoxy, sec-butoxy, isobutoxy, tert-butoxy, pentyloxy, isopentyloxy, neopentyloxy and hexyloxy.

The (C1-C6)alcohol means a (C1-C6)alkyl-OH, wherein the (C1-C6)alkyl moiety has the same meaning as defined above. Examples of the (C1-C6)alcohol include, but are not limited to, methanol, ethanol, propanol (i.e., 1-propanol), 2-propanol, butanol (i.e., 1-butanol), sec-butanol, isobutanol, tert-butanol, pentanol (i.e., 1-pentanol), sec-amyl alcohol, 3-pentanol, 2-methyl-1-butanol, isoamyl alcohol, tert-amyl alcohol, hexanol (i.e., 1-hexanol) and cyclohexanol. Polyols having 1 to 6 carbons (e.g., diols and triols) such as ethylene glycol, propylene glycol and glycerol are equivalents of (C1-C6)alcohols.

The (C1-C4)alcohol means a (C1-C4)alkyl-OH, wherein the (C1-C4)alkyl moiety has the same meaning as defined above. Examples of the (C1-C4)alcohol include, but are not limited to, methanol, ethanol, propanol (i.e., 1-propanol), 2-propanol, butanol, sec-butanol, isobutanol and tert-butanol. Polyols having 1 to 4 carbons (e.g., diols and triols) such as ethylene glycol, propylene glycol and glycerol are equivalents of (C1-C4)alcohols.

The (C2-C5)alkanenitrile means (C1-C4)alkyl-CN, wherein the (C1-C4)alkyl moiety means a linear or branched alkyl having 1 to 5 carbon atoms; examples of the (C1-C5)alkyl include appropriate examples among the examples of the (C1-C6)alkyl described above. Examples of the (C2-C5)alkanenitrile include, but are not limited to, acetonitrile and propionitrile. Herein, the (C2-C5)alkanenitrile is also referred to as C2-C5 alkanenitrile. C2 alkanenitrile is acetonitrile. In other words, acetonitrile is ethanenitrile in accordance with the IUPAC nomenclature and is a C2 alkanenitrile having two carbon atoms. Similarly, propionitrile is a C3 alkanenitrile.

Examples of the (C1-C4)alkyl (C1-C4)carboxylates include, but are not limited to, ethyl acetate, propyl acetate, isopropyl acetate, butyl acetate and isomers thereof, ethyl propionate, propyl propionate, isopropyl propionate, butyl propionate and isomers thereof, and preferably ethyl acetate, propyl acetate, isopropyl acetate, butyl acetate and isomers thereof. Herein, (C1-C4)alkyl (C1-C4)carboxylate is also referred to as C1-C4 alkyl C1-C4 carboxylate.

Examples of the N,N-di((C1-C4)alkyl) (C1-C4)alkanamides include, but are not limited to, N,N-dimethylformamide, N,N-dimethylacetamide, N,N-diethylformamide and N,N-diethylacetamide, and preferably N,N-dimethylformamide and N,N-dimethylacetamide. Herein, N,N-di((C1-C4)alkyl) (C1-C4)alkanamide is also referred to as N,N-di(C1-C4 alkyl)C1-C4 alkanamide. N,N-di(C1 alkyl)C1 alkanamide is N,N-dimethylformamide. N,N-di(C1 alkyl)C2 alkanamide is N,N-dimethylacetamide.

The (C1-C4)alkanoic acid means (C1-C3)alkyl-COOH and formic acid (HCOOH), i.e., (C1-C3)alkyl-C(═O)—OH and H—C(═O)—OH (wherein a (C0-C4)alkyl moiety is understood in accordance with definition similar to that employed herein). Examples of the (C1-C4)alkanoic acid include, but are not limited to, acetic acid and propionic acid, and preferably acetic acid. Herein, a (C1-C4)carboxylic acid is written also as a C1-C4 carboxylic acid.

The (C2-C4)alkanoic acid substituted with 1 to 7 fluorine atoms means a (C1-C3)alkyl-COOH wherein 1 to 7 hydrogens present on a (C1-C3)alkyl are substituted with fluorine atoms. Examples of the (C2-C4)alkanoic acid substituted with 1 to 7 fluorine atoms include, but are not limited to, monofluoroacetic acid, difluoroacetic acid, trifluoroacetic acid, and pentafluoropropionic acid, and preferably trifluoroacetic acid. The (C2-C4)alkanoic acid substituted with 1 to 7 fluorine atoms is written also as a C2-C4 alkanoic acid substituted with 1 to 7 fluorine atoms.

Examples of the (C1-C4)alkyl (C1-C4)alkyl ketones include, but are not limited to, acetone, methyl ethyl ketone (MEK), methyl isopropyl ketone (MIPK) and methyl isobutyl ketone (MIBK). Herein, (C1-C4) alkyl (C1-C4) alkyl ketone is also referred to as C1-C4 alkyl C1-C4 alkyl ketone.

Examples of the (C1-C4)dihaloalkanes include, but are not limited to, dichloromethane and 1,2-dichloroethane. Herein, (C1-C4)dihaloalkane is also referred to as C1-C4 dihaloalkane.

The cyclic hydrocarbon group means a cyclic group which is monocyclic or multicyclic, wherein all of the ring-constituting atoms are carbon atoms. In one embodiment, examples of the cyclic hydrocarbon group include, but are not limited to, a 3- to 14-membered (preferably 5- to 14-membered, more preferably 5- to 10-membered) cyclic hydrocarbon group which is aromatic or non-aromatic and is monocyclic, bicyclic or tricyclic. In another embodiment, examples of the cyclic hydrocarbon group include, but are not limited to, a 4- to 8-membered (preferably 5- to 6-membered) cyclic hydrocarbon group which is aromatic or non-aromatic and is monocyclic or bicyclic (preferably monocyclic). Examples of the cyclic hydrocarbon group include, but are not limited to, cycloalkyls and aryls. Examples of the cycloalkyl include the examples of the (C3-C6)cycloalkyl described above. The aryls are aromatic cyclic groups among the cyclic hydrocarbon groups as defined above. Examples of the aryl include the examples of the (C6-C10)aryl described above. The cyclic hydrocarbon group as defined or exemplified above may include a non-condensed cyclic group (e.g., a monocyclic group or a spirocyclic group) and a condensed cyclic group, when possible. The cyclic hydrocarbon group as defined or exemplified above may be unsaturated, partially saturated or saturated, when possible. The cyclic hydrocarbon group as defined or exemplified above is also referred to as a carbocyclic ring group. The carbocyclic ring is a ring which corresponds to the cyclic hydrocarbon group as defined or exemplified above. Examples of the carbocyclic ring include, but are not limited to, cyclopropane, cyclobutane, cyclopentane, cyclohexane, cyclopentene and cyclohexene.

Herein, there are no particular limitations on the “substituent(s)” for the phrase “optionally substituted with one or more substituents” as long as they are chemically acceptable and exhibit the effects of the present invention.

Herein, examples of the “substituent(s)” for the phrase “optionally substituted with one or more substituent(s)” include, but are not limited to, one or more substituents (preferably 1 to 3 substituents) selected independently from Substituent Group (a).

Substituent Group (a) is a group consisting of a halogen atom; a nitro group, a cyano group, a hydroxy group, an amino group, (C1-C6)alkyl, (C1-C6)haloalkyl, (C3-C6)cycloalkyl, (C2-C6)alkenyl, (C2-C6)alkynyl, (C1-C6)alkoxy, phenyl and phenoxy.

In addition, one or more substituents (preferably 1 to 3 substituents) selected independently from Substituent Group (a) may each independently be substituted with one or more substituents (preferably 1 to 3 substituents) selected independently from Substituent Group (b). In this context, Substituent Group (b) is the same as Substituent Group (a).

Examples of the “(C1-C6)alkyl optionally substituted with one or more substituents” include, but are not limited to, (C1-C6)haloalkyl, (C1-C4)perfluoroalkyl and (C1-C4)alkyl optionally substituted with 1 to 9 fluorine atoms.

Examples of the (C1-C4)alkyl optionally substituted with 1 to 9 fluorine atoms include, but are not limited to, fluoromethyl (i.e., —CH₂F), difluoromethyl (i.e., —CHF₂), trifluoromethyl (i.e., —CF₃), 2-fluoroethyl, 2,2,2-trifluoroethyl, pentafluoroethyl, 3-fluoropropyl, 2,2,3,3,3-pentafluoropropyl, 2,2,2-trifluoro-1-trifluoromethylethyl, heptafluoropropyl, 1,2,2,2-tetrafluoro-1-trifluoromethylethyl, 4-fluorobutyl, 2,2,3,3,4,4,4-heptafluorobutyl, nonafluorobutyl, 1,1,2,3,3,3-hexafluoro-2 trifluoromethylpropyl and 2,2,2-trifluoro-1,1-di(trifluoromethyl)ethyl.

Herein, the phrase “as described herein” and similar phrases used when referring to substituents (for example, R¹, R², R³, R⁴, R⁵, X¹, X², X³, X⁴ and X⁵) incorporate by reference all definitions of the substituents and, if any, all of their examples, preferred examples, more preferred examples, further preferred examples and particularly preferred examples in this specification.

As used herein, the non-limiting term “comprise(s)/comprising” can each optionally be replaced by the limiting phrase “consist(s) of/consisting of”.

Unless otherwise stated, all technical and scientific terms used herein have the same meaning as commonly understood by a person skilled in the art to which the present disclosure belongs.

Unless otherwise indicated, it is understood that numbers used herein to express characteristics such as quantities, sizes, concentrations, and reaction conditions are modified by the term “about”. In some embodiments, disclosed numerical values are interpreted applying the reported number of significant digits and conventional rounding techniques. In some embodiments, disclosed numerical values are interpreted as containing certain errors necessarily resulting from the standard deviation found in their respective testing measurements.

(Step i-a)

The step i-a will be described.

In the step i-a, the compound of the formula (7) is produced by reacting a compound of the formula (1) with a compound of the formula (2) in the presence of a base:

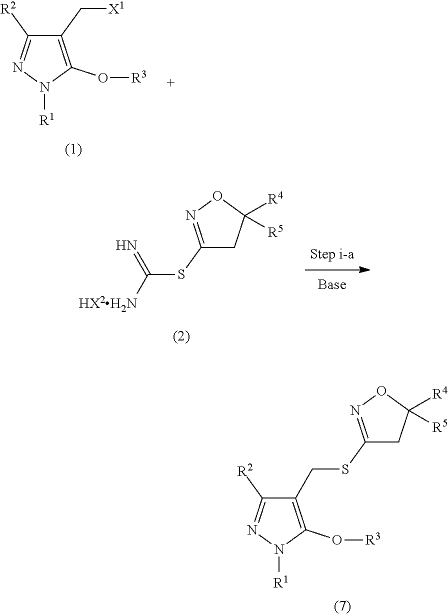

wherein in the formula (1), the formula (2) and the formula (7), R¹, R², R³, R⁴, R⁵, X¹ and X² are as defined above.

The reaction in the step i-a is a condensation reaction.

(Raw Material in Step i-a; Compound of Formula (1))

A compound of the formula (1) is used as a raw material in the step i-a. The compound of the formula (1) may be a known compound or may be produced from a known compound according to a known process.

WO 2007/094225 A1 (Patent Document 5) is summarized as follows. For example, WO 2007/094225 A1 (Patent Document 5) discloses that a pyrazole derivative FMTP is produced from an acetoacetic acid ester derivative as shown in the following scheme. As shown in Example 1-1, a compound of the formula (1-a) can be produced by chlorinating this pyrazole derivative.

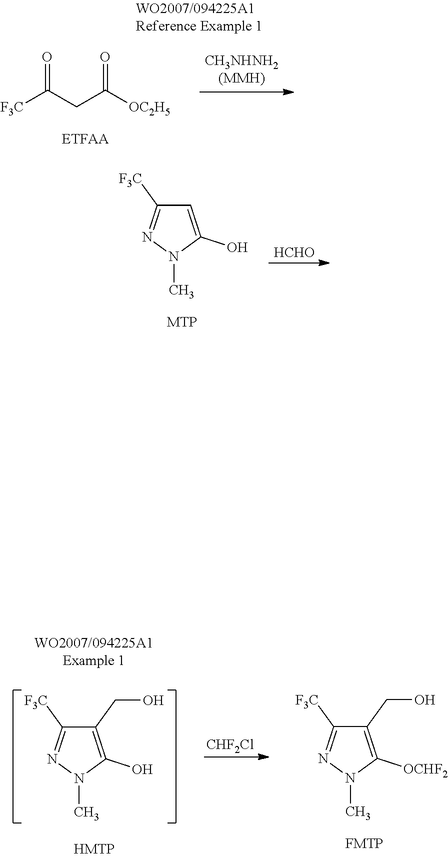

In the formula (1), R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents.

In the formula (1), X¹ is a leaving group. X¹ in the formula (1) may be any atom or atomic group as long as it functions as a leaving group in the reaction in the step i-a.

From the viewpoints of yield, availability, price, usefulness of the product, etc., preferred examples of R¹ in the formula (1) include (C1-C6)alkyls optionally substituted with one or more substituents, more preferably (C1-C6)alkyls, further preferably (C1-C4)alkyls, and particularly preferably methyl.

From the same viewpoints as described above, preferred examples of R² in the formula (1) include (C1-C6)alkyls optionally substituted with one or more substituents, more preferably (C1-C6)haloalkyls, further preferably (C1-C4)perfluoroalkyls, and particularly preferably trifluoromethyl.

From the same viewpoints as described above, preferred examples of R³ in the formula (1) include (C1-C6)alkyls optionally substituted with one or more substituents, more preferably (C1-C6)haloalkyls, further preferably (C1-C4)alkyls optionally substituted with 1 to 9 fluorine atoms, and particularly preferably difluoromethyl.

From the viewpoint of yield, availability, price, etc., preferred examples of X¹ in the formula (2) include halogen atoms, (C1-C4)alkylsulfonyloxys, (C1-C4)haloalkylsulfonyloxys, (C1-C4)alkyls, or benzenesulfonyloxy optionally having a halogen atom, more preferably a chlorine atom, a bromine atom, an iodine atom, methanesulfonyloxy, ethanesulfonyloxy, trifluoromethanesulfonyloxy, benzenesulfonyloxy, p-toluenesulfonyloxy and p-chlorobenzenesulfonyloxy, further preferably a chlorine atom and a bromine atom, and particularly preferably a chlorine atom.

Other processes for preparing the compound of the formula (1) are described in Examples 13 and 14 of WO 2004/013106 A1 (Patent Document 2), which are as follows:

### WO2004/013106A1. Example 13

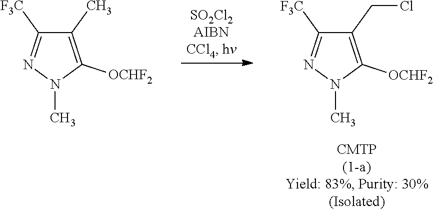 [SMILES: Cc1c(C(F)(F)F)nn(C)c1OC(F)F ; Cn1nc(C(F)(F)F)c(CCl)c1OC(F)F]

### WO2004/013106A1 Example 14

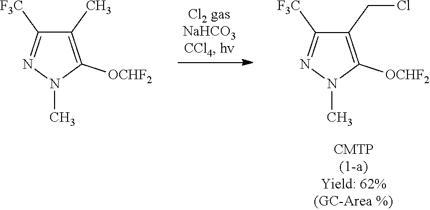

In the formula (1), R¹, R², R³ and X¹ are as defined above. In the formula (1), examples, preferred examples, more preferred examples, and particularly preferred examples of R¹, R², R³, and X¹ are as described above.

A particularly preferred specific example of the compound of the formula (1) is as follows:

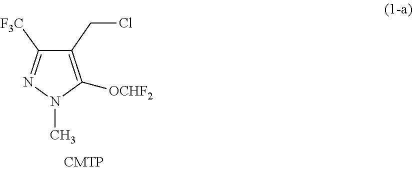 [SMILES: Cn1nc(C(F)(F)F)c(CCl)c1OC(F)F]

Specific examples and particularly preferred specific examples of the compound of the formula (1) are as described above.

(Raw Material in Step i-a; Compound of Formula (2))

A compound of the formula (2) is used as a raw material in the step i-a.

The compound of the formula (2) may be a known compound or may be produced from a known compound according to a known process. For example, the preparation of the compound of the formula (2) can be performed by the processes described in WO 2006/068092 A1 (Patent Document 6), JP 2013-512201 A (Patent Document 7) and WO 2019/131715 A1 (Patent Document 8), or by processes similar thereto. JP 2013-512201 A, paragraph 0004 (US 2012/264947 A1, paragraph 0007) (Patent Document 7) disclose a process for producing the raw material used in the process described in WO 2006/068092 A1 (Patent Document 6) by citing JP 2008-001597 A and WO 2006/038657 A1. These are summarized in the following scheme.

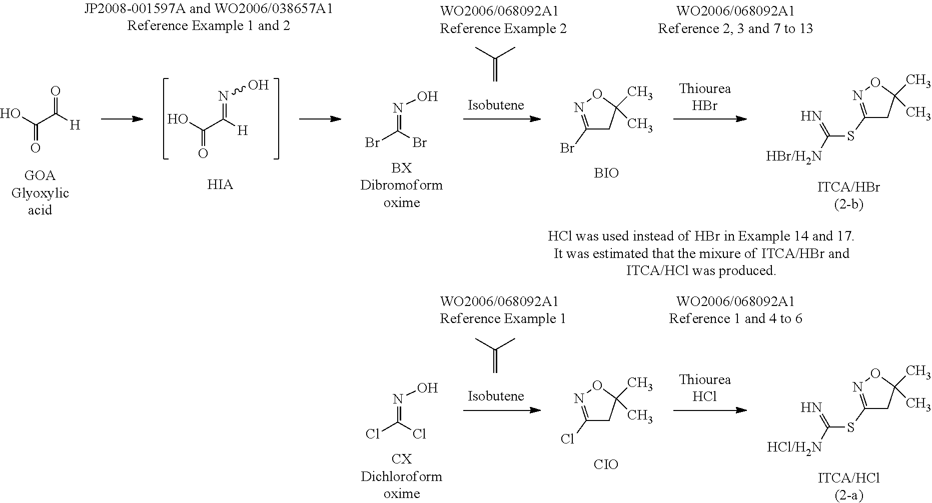

In the formula (2), R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents; or R⁴ and R⁵, together with the carbon atom to which they are attached, form a 4- to 12-membered carbocyclic ring, wherein the carbocyclic ring is optionally substituted with one or more substituents.

From the viewpoints of yield, availability, price, usefulness of the product, etc., preferred examples of R⁴ and R⁵ in the formula (2) each independently include (C1-C6)alkyls optionally substituted with one or more substituents, more preferably (C1-C6)alkyls, further preferably (C1-C4)alkyls, and particularly preferably methyl.

X² in the formula (2) is an atom or an atomic group that forms an acid. Thus, HX² is an acid.

From the viewpoint of yield, availability, price, usefulness of the product, etc., preferred examples of X² in the formula (2) include:
halogen atoms, a sulfate group, a hydrogen sulfate group, a phosphate group, a monohydrogen phosphate group, a dihydrogen phosphate group, (C1-C4)alkylsulfonyloxys, (C1-C4)haloalkylsulfonyloxys, benzenesulfonyloxys optionally having an (C1-C4)alkyl or a halogen atom, and mixtures of two or more (preferably two or three, more preferably two) thereof, more preferably a chlorine atom, a bromine atom, an iodine atom, a sulfate group, a hydrogen sulfate group, a phosphate group, a monohydrogen phosphate group, a dihydrogen phosphate group, methanesulfonyloxy, ethanesulfonyloxy, trifluoromethanesulfonyloxy, benzenesulfonyloxy, p-toluenesulfonyloxy, p-chlorobenzenesulfonyloxy and mixtures of two or more (preferably two or three, more preferably two) thereof, still more preferably a chlorine atom, a bromine atom, a sulfate group, a hydrogen sulfate group, a phosphate group, a monohydrogen phosphate group, methanesulfonyloxy, p-toluenesulfonyloxy, and mixtures of two or more (preferably two or three, more preferably two) thereof, and particularly preferably a chlorine atom, a bromine atom and a mixture thereof.

Particularly preferred specific examples of the compound of the formula (2) are the following compounds (2-a), (2-b), and a mixture thereof.

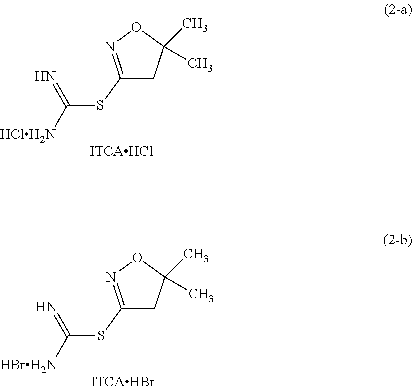

In addition, when “X_{2H}” is a polyvalent acid such as sulfuric acid or phosphoric acid, it is within the scope of the present invention that the ratio between “X2 of the acid moiety” and “(4,5-dihydroisoxazolo-3-yl)thiocarboxamidine moiety in the following formula (2-1)” can be a ratio corresponding to all possible valences of the polyvalent acid.

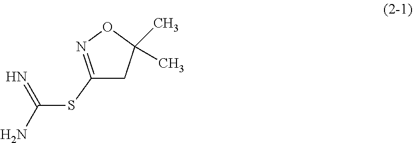

In other words, for example, the compound of the following formula (2-c) is an equivalent of the compound of the formula (2).

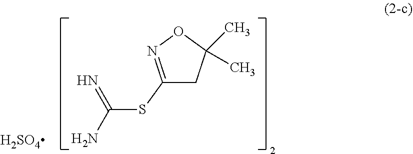

In the reaction in the step i-a, it was presumed that the isothiouronium group in the compound of the formula (2) produced the corresponding thiol group and/or a salt thereof (e.g., generally —S⁻N⁺ or —S⁻K⁺) and/or an analog thereof. Compounds having thiol groups and/or salts thereof and/or analogs thereof corresponding to the compounds of the formula (2) are equivalents of the compounds of the formula (2), and processes using the equivalents are within the scope of the present invention as defined by the appended claims.

(Raw Material in Step i-a: Amount of Compound of Formula (2) Used)

The amount of the formula (2) used in the step i-a may be any amount as long as the reaction proceeds. The amount of the formula (2) used in the step i-a may be appropriately adjusted by a person skilled in the art. However, from the viewpoint of yield, suppression of by-products, economic efficiency, etc., the amount of the compound of the formula (2) used in the step i-a is, for example, 0.5 to 2.0 mol or more, preferably 0.8 to 1.5 mol, more preferably 1.0 to 1.5 mol, and still more preferably 1.0 to 1.1 mol, based on 1 mol of the compound of the formula (1) (raw material).

(Product in Step i-a; Compound of Formula (7))

The product in the step i-a is a compound of the formula (7) corresponding to the compound of the formula (1) and the compound of the formula (2) used as raw materials.

In the formula (7), R¹, R² and R³ are as defined in the formula (1). In the formula (7), R⁴ and R⁵ are as defined in the formula (2). In the formula (7), examples, preferred examples, more preferred examples, and particularly preferred examples of R¹, R², R³, R⁴ and R⁵ are the same as those in the formula (1) and the formula (2) described above, respectively.

A particularly preferred specific example of the compound of the formula (7) is as follows:

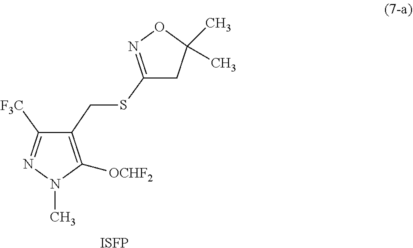

(Base in Step i-a)

The reaction in the step i-a is performed in the presence of a base. The base may be any base as long as the reaction proceeds. Examples of the base in the step i-a include, but are not limited to, the following:
alkali metal hydroxides (e.g., lithium hydroxide, sodium hydroxide and potassium hydroxide), alkaline earth metal hydroxides (e.g., magnesium hydroxide, calcium hydroxide and barium hydroxide), alkali metal carbonates (e.g., lithium carbonate, sodium carbonate, potassium carbonate and cesium carbonate), alkaline earth metal carbonates (e.g., magnesium carbonate and calcium carbonate), alkali metal hydrogen carbonates (e.g., lithium hydrogen carbonate, sodium hydrogen carbonate and potassium hydrogen carbonate), alkaline earth metal hydrogen carbonates (e.g., calcium hydrogen carbonate), phosphate salts (e.g., sodium phosphate, potassium phosphate and calcium phosphate), hydrogen phosphate salts (e.g., sodium hydrogen phosphate, potassium hydrogen phosphate and calcium hydrogen phosphate), amines (e.g., triethylamine, tributylamine, diisopropylethylamine, 1,8-diazabicyclo[5.4.0]-7-undec-7-ene (DBU), 1,4-diazabicyclo[2.2.2]octane (DABCO), pyridine and 4-(dimethylamino)-pyridine (DMAP)), ammonia, and a mixture thereof.

From the viewpoint of yield, suppression of by-products, economic efficiency, etc., preferred examples of the base in the step i-a include alkali metal hydroxides, alkali metal carbonates, alkali metal hydrogen carbonates, and a mixture thereof, more preferably alkali metal hydroxides, alkali metal carbonates, and a mixture thereof, and further preferably alkali metal hydroxides.

From the same viewpoint as described above, preferred specific examples of the base in the step i-a include lithium hydroxide, sodium hydroxide, potassium hydroxide, lithium carbonate, sodium carbonate, potassium carbonate, lithium hydrogen carbonate, sodium hydrogen carbonate, potassium hydrogen carbonate and a mixture thereof, more preferably lithium hydroxide, sodium hydroxide, potassium hydroxide, lithium carbonate, sodium carbonate, potassium carbonate and a mixture thereof, still more preferably sodium hydroxide, potassium hydroxide, sodium carbonate, potassium carbonate and a mixture thereof, further preferably sodium hydroxide, potassium hydroxide and a mixture thereof, and particularly preferably sodium hydroxide.

The base in the step i-a may be used singly or in a combination of two or more kinds thereof in any ratio. The base in the step i-a may be in any form as long as the reaction proceeds. Examples of the form of the base in the step i-a include a base-only solid and an aqueous solution with any concentration. Specific examples of the form of the base include, but are not limited to, a flake, a pellet, a bead, a powder and a 10 to 50% aqueous solution, and preferably a 20 to 50% aqueous solution (e.g., a 25% aqueous sodium hydroxide solution and a 48% aqueous sodium hydroxide solution, preferably a 48% aqueous sodium hydroxide solution). The form of the base in the step i-a can be appropriately selected by a person skilled in the art.

The amount of the base used in the step i-a may be any amount as long as the reaction proceeds. The amount of the base used in the step i-a can be appropriately adjusted by a person skilled in the art. However, from the viewpoint of yield, suppression of by-products, economic efficiency, etc., in one embodiment, the amount of the base used in the step i-a is, for example, 5 to 10 mol, preferably 5 to 8 mol, more preferably 5 to 7 mol, and still more preferably 5 to 6 mol, based on 1 mol of the compound of the formula (1) (raw material). In another embodiment, for example, the amount is 1 to 15 mol, preferably 1 to 10 mol, more preferably 2 to 9 mol, still more preferably 4 to 8 mol, and further preferably 5 to 6 mol, based on 1 mol of the compound of the formula (1) (raw material).

(Reaction Solvent in Step i-a)

From the viewpoint of allowing the reaction to smoothly proceed, the reaction in the step i-a is preferably performed in the presence of a solvent.

The solvent in the reaction in the step i-a may be any solvent as long as the reaction proceeds.

Examples of the solvent in the reaction in the step i-a include, but are not limited to, the following:
aromatic hydrocarbon derivatives (e.g., benzene, toluene, xylenes, chlorobenzene, dichlorobenzenes, trichlorobenzenes and nitrobenzene), halogenated aliphatic hydrocarbons (e.g., dichloromethane and 1,2-dichloroethane (EDC)), alcohols (e.g., methanol, ethanol, propanol, 2-propanol, butanol, sec-butanol, isobutanol and tert-butanol (tert-butanol being also referred to as tert-butyl alcohol), pentanol, sec-amyl alcohol, 3-pentanol, 2-methyl-1-butanol, isoamyl alcohol, tert-amyl alcohol, hexanol and cyclohexanol), nitriles (e.g., acetonitrile and propionitrile), carboxylic acid esters (e.g., methyl acetate, ethyl acetate, propyl acetate, isopropyl acetate, butyl acetate and isomers thereof, and pentyl acetate and isomers thereof), ethers (e.g., tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME) and diglyme), ketones (e.g., acetone, methyl ethyl ketone (MEK), methyl isopropyl ketone (MIPK) and methyl isobutyl ketone (MIBK)), amides (e.g., N,N-dimethylformamide (DMF), N,N-dimethylacetamide (DMAC) and N-methylpyrrolidone (NMP)), ureas (e.g., N,N′-dimethylimidazolidinone (DMI) and tetramethylurea), sulfoxides (e.g., dimethyl sulfoxide (DMSO)), sulfones (e.g., sulfolane), water, and any combination thereof in any ratio. “2-Propanol” is also referred to as “isopropyl alcohol” or “isopropanol”.

However, from the viewpoint of yield, suppression of by-products, economic efficiency, etc., preferred examples of the solvent in the reaction in the step i-a include the following: combinations of one or more (preferably one or two, more preferably one) organic solvents selected from aromatic hydrocarbon derivatives, halogenated aliphatic hydrocarbons, alcohols, nitriles, carboxylic acid esters, ethers, ketones, amides, ureas, sulfoxides, and sulfones, with a water solvent in any ratio.

More preferred examples of the solvent in the reaction in the step i-a include combinations of one or more (preferably one or two, more preferably one) organic solvents selected from alcohols, nitriles, carboxylic acid esters, ethers, amides and sulfones with a water solvent in any ratio.

More preferred examples of the solvent in the reaction in the step i-a include combinations of one or more (preferably one or two, more preferably one) organic solvents selected from alcohols, nitriles, carboxylic acid esters, ethers and amides with a water solvent in any ratio.

More preferred examples of the solvent in the reaction in the step i-a include combinations of one or more (preferably one or two, more preferably one) organic solvents selected from alcohols, nitriles, carboxylic acid esters and amides with a water solvent in any ratio.

More preferred examples of the solvent in the reaction in the step i-a include combinations of one or more (preferably one or two, more preferably one) organic solvents selected from alcohols, nitriles and carboxylic acid esters with a water solvent in any ratio.

Still more preferred examples of the solvent in the reaction in the step i-a include combinations of one or more (preferably one or two, more preferably one) organic solvents selected from nitriles and carboxylic acid esters with a water solvent in any ratio.

In one embodiment, particularly preferred examples of the solvent in the reaction in the step i-a include combinations of nitriles with a water solvent in any ratio.

In another embodiment, particularly preferred examples of the solvent in the reaction in the step i-a include combinations of carboxylic acid esters with a water solvent in any ratio.

From the same viewpoint as described above, preferred specific examples of the solvent in the reaction in the step i-a include combinations of one or more (preferably one or two, more preferably one) organic solvents selected from toluene, xylenes, chlorobenzene, dichlorobenzenes, dichloromethane, 1,2-dichloroethane, methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane with a water solvent in any ratio.

From the same viewpoint as described above, more preferred specific examples of the solvent in the reaction in the step i-a include combinations of one or more (preferably one or two, more preferably one) organic solvents selected from toluene, xylenes, chlorobenzene, dichlorobenzenes, dichloromethane, 1,2-dichloroethane, methanol, ethanol, propanol, 2-propanol, butanol, sec-butanol, isobutanol, tert-butanol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane with a water solvent in any ratio.

From the same viewpoint as described above, still more preferred specific examples of the solvent in the reaction in the step i-a include combinations of one or more (preferably one or two, more preferably one) organic solvents selected from toluene, xylenes, chlorobenzene, dichlorobenzenes, dichloromethane, 1,2-dichloroethane, methanol, ethanol, 2-propanol, butanol, tert-butanol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane with a water solvent in any ratio.

More preferred specific examples of the solvent in the reaction in the step i-a include combinations of one or more (preferably one or two, more preferably one) organic solvents selected from methanol, ethanol, 2-propanol, butanol, tert-butanol, acetonitrile, ethyl acetate, propyl acetate, isopropyl acetate, butyl acetate and isomers thereof with a water solvent in any ratio.

Still more preferred specific examples of the solvent in the reaction in the step i-a include combinations of one or more (preferably one or two, more preferably one) organic solvents selected from butanol, acetonitrile, ethyl acetate, propyl acetate, isopropyl acetate and butyl acetate with a water solvent in any ratio.

Further preferred specific examples of the solvent in the reaction in the step i-a include combinations of one or more (preferably one or two, more preferably one) organic solvents selected from acetonitrile, ethyl acetate, isopropyl acetate and butyl acetate with a water solvent in any ratio.

Still further preferred specific examples of the solvent in the reaction in the step i-a include combinations of one or two (preferably one) organic solvents selected from acetonitrile and butyl acetate with a water solvent in any ratio.

In one embodiment, a particularly preferred specific example of the solvent in the reaction in the step i-a includes a combination of an acetonitrile solvent with a water solvent in any ratio.

In another embodiment, a particularly preferred specific example of the solvent in the reaction in the step i-a includes a combination of a butyl acetate solvent with a water solvent in any ratio.

In either case, the solvent may be in a single layer or may be separated into two layers as long as the reaction proceeds.

The amount of the solvent used in the reaction in the step i-a will now be described. The “total amount of the solvent used in the reaction” is the sum total of the amounts of all the organic solvents and the amount of the water solvent used in the reaction. The organic solvent and the water solvent used in the working-up (e.g., isolation and purification) after the reaction are not included. The “organic solvent” used in the reaction includes the organic solvent in the raw material solution and that in the reactant solution. The “water solvent” used in the reaction includes the water in the raw material solution and that in the reactant solution (e.g., water in a 48% aqueous sodium hydroxide solution).

The total amount of the solvent used in the reaction in the step i-a is not particularly limited as long as the reaction system can be sufficiently stirred. However, from the viewpoint of yield, suppression of by-products, economic efficiency, etc., in one embodiment, the total amount of the solvent used in the reaction in the step i-a is, for example, 0.1 to 10 L (liters), preferably 0.5 to 5 L, more preferably 1 to 5 L, still more preferably 1 to 3 L, and further preferably 1 to 2 L, based on 1 mol of the compound of the formula (1) (raw material). In another embodiment, the total amount of the solvent used in the reaction in the step i-a is, for example, 1.5 to 3.0 L (liters), preferably 1.5 to 2.5 L, and more preferably 1.5 to 2.0 L, based on 1 mol of the compound of the formula (1) (raw material). In still another embodiment, the total amount of the solvent used in the reaction in the step iis, for example, 1.7 to 3.0 L (liters), preferably 1.7 to 2.5 L, and more preferably 1.7 to 2.0 L, based on 1 mol of the compound of the formula (1) (raw material).

From the same viewpoint as described above, in one embodiment, the amount of the organic solvent used in the reaction in the step i-a is, for example, 0 (zero) to 5 L (liters), preferably 0.4 to 2.0 L, more preferably 0.5 to 1.5 L, still more preferably 0.6 to 1.0 L, and further preferably 0.7 to 0.9 L, based on 1 mol of the compound of the formula (1) (raw material). In another embodiment, the amount of the organic solvent used in the reaction in the step i-a is, for example, 0.1 to 5 L (liters), preferably 0.3 to 2.0 L, more preferably 0.4 to 1.5 L, still more preferably 0.5 to 1.0 L, and further preferably 0.6 to 0.8 L, based on 1 mol of the compound of the formula (1) (raw material).

From the same viewpoint as described above, the amount of the water solvent used in the reaction in the step i-a is, for example, 0.1 to 5 L (liters), preferably 0.5 to 2.0 L, more preferably 0.5 to 1.5 L, still more preferably 0.7 to 1.4 L, and further preferably 0.9 to 1.2 L, based on 1 mol of the compound of the formula (1) (raw material).

When a combination of two or more organic solvents is used, the ratio of the two or more organic solvents may be any ratio as long as the reaction proceeds.

When a combination of an organic solvent and a water solvent is used, the ratio of the organic solvent and the water solvent may be any ratio as long as the reaction proceeds.

(Reaction Temperature in Step i-a)

The reaction temperature in the step i-a is not particularly limited. However, from the viewpoint of yield, suppression of by-products, economic efficiency, etc., the reaction temperature in the step i is, for example, −10 (minus 10)° C. to 100° C., preferably −10° C. to 70° C., more preferably −10° C. to 50° C., still more preferably 0 (zero)° C. to 40° C., further preferably 0° C. to 30° C., and further preferably 0° C. to 25° C.

(Reaction Time in Step i-a)

The reaction time in the step i-a is not particularly limited. However, from the viewpoint of yield, suppression of by-products, economic efficiency, etc., in one embodiment, the reaction time in the step i-a is, for example, 4 hours to 48 hours, preferably 4 hours to 24 hours, more preferably 4 hours to 18 hours, and still more preferably 4 hours to 12 hours. In another embodiment, the reaction time in the step i-a is, for example, 1 hour to 48 hours, preferably 1 hour to 24 hours, more preferably 3 hours to 18 hours, and still more preferably 3 hours to 12 hours. However, the reaction time can be adjusted appropriately by a person skilled in the art.

(Adding Method in Step i-a)

The order of adding the compound of the formula (1), the compound of the formula (2), the base, the solvent, etc. is not particularly limited. As long as the reaction proceeds, the addition order thereof may be any order. For example, the base may be added dropwise to a mixture comprising the compound of the formula (1), the compound of the formula (2) and the solvent in a reaction vessel. As another example, the compound of the formula (1) may be added dropwise to a reaction vessel after adding the compound of the formula (2), the base and the solvent thereto. As still another example, the compound of the formula (1) and the compound of the formula (2) may be successively added dropwise to a reaction vessel after adding the base and the solvent thereto.

(Working-up in Step i-a; Isolation and/or Purification)

The compound of the formula (7), especially the compound (7-a), which is the product in the step i-a, can be used as a raw material in the step ii. The compound of the general formula (7) obtained in the step i-a may be isolated and/or purified and then used in the next step, or may be used in the next step without being isolated. Whether or not to perform the working-up (isolation and/or purification) can be appropriately determined by a person skilled in the art according to the purpose and situation.

The compounds of the formula (7), especially the compound (7-a), which is the target product in the step i-a, can be isolated and purified from the reaction mixture by any of methods known to a person skilled in the art (e.g., extraction, washing, crystallization including recrystallization, crystal washing and/or other procedures) and improved methods thereof, and any combination thereof.

(Step i-b)

The step i-b will now be described.

The step i-b is a step of producing the compound of the formula (7) by reacting a compound of the formula (4) with a compound of the formula (3) in the presence of a base.

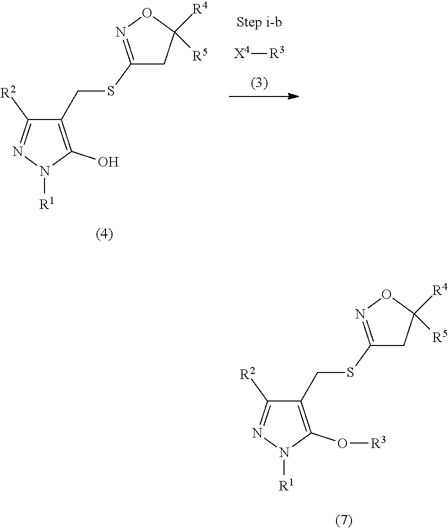

wherein in the formula (3), the formula (4), and the formula (7), R¹, R², R³, R⁴, R⁵, and X⁴ are as defined above.

(Raw Material in Step i-b: Compound of Formula (4))

A compound of the formula (4) is used as a raw material in the step i-b. The compound of the formula (4) may be a known compound or may be produced from a known compound according to a known process. For example, the preparation of the compound of the formula (4) is described in Reference Example 1 of WO 2005/105755 A1 (Patent Document 4), which is as follows:

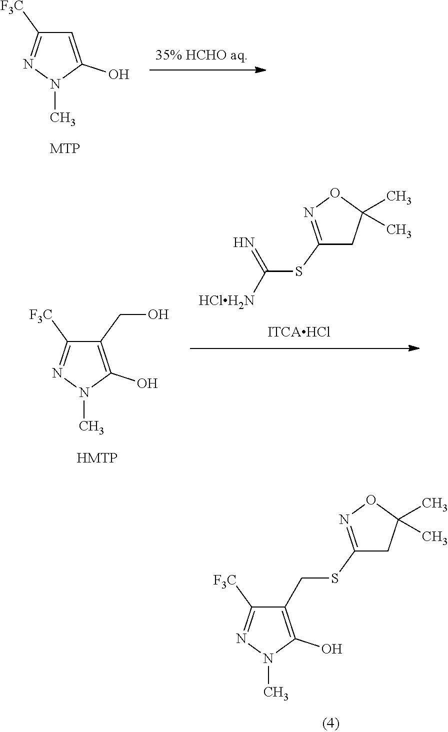

In the formula (4), R¹, R², R³, R⁴ and R⁵ are as defined above. In the formula (4), examples, preferred examples, more preferred examples, and particularly preferred examples of R¹, R², R³, R⁴ and R⁵ are as described above.

Particularly preferred specific examples of the compound of the formula (4) are as follows:

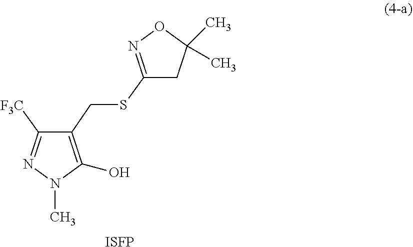

(Raw Material in Step i-b: Compound of Formula (3))

A compound of the formula (3) is used as a raw material in the step i-b. The compound of the formula (3) may be a known compound or may be produced from a known compound according to a known process.

In the formula (3), R³ is as defined above, and X⁴ is a leaving group. X⁴ in the formula (3) may be any atom or atomic group as long as it functions as a leaving group in the reaction in the step i-b.

From the viewpoint of yield, availability, price, etc., preferred examples of X⁴ in the formula (3) include halogen atoms, (C1-C4)alkylsulfonyloxy, (C1-C4)haloalkylsulfonyloxy, (C1-C4)alkyl, and benzenesulfonyloxy optionally having a halogen atom, more preferably a chlorine atom, a bromine atom, an iodine atom, methanesulfonyloxy, ethanesulfonyloxy, trifluoromethanesulfonyloxy, benzenesulfonyloxy, p-toluenesulfonyloxy and p-chlorobenzenesulfonyloxy, further preferably a chlorine atom and a bromine atom, and particularly preferably a chlorine atom.

In the formula (3), R³ and X⁴ are as defined above. In the formula (3), examples, preferred examples, more preferred examples, and particularly preferred examples of R³ and X⁴ are as described above.

A particularly preferred specific example of the compound of the formula (3) is chlorodifluoromethane.

(Base in Step i-b)

The reaction in the step i-b is performed in the presence of a base. The base may be any base as long as the reaction proceeds. Examples of the base in the step i-b include, but are not limited to, the following: alkali metal hydroxides (e.g., lithium hydroxide, sodium hydroxide and potassium hydroxide), alkaline earth metal hydroxides (e.g., magnesium hydroxide, calcium hydroxide and barium hydroxide), alkali metal carbonates (e.g., lithium carbonate, sodium carbonate, potassium carbonate and cesium carbonate), alkaline earth metal carbonates (e.g., magnesium carbonate and calcium carbonate), alkali metal hydrogen carbonates (e.g., lithium hydrogen carbonate, sodium hydrogen carbonate and potassium hydrogen carbonate), alkaline earth metal hydrogen carbonates (e.g., calcium hydrogen carbonate), phosphate salts (e.g., sodium phosphate, potassium phosphate and calcium phosphate), hydrogen phosphate salts (e.g., sodium hydrogen phosphate, potassium hydrogen phosphate and calcium hydrogen phosphate), amines (e.g., triethylamine, tributylamine, diisopropylethylamine, 1,8-diazabicyclo[5.4.0]-7-undec-7-ene (DBU), 1,4-diazabicyclo[2.2.2]octane (DABCO), pyridine and 4-(dimethylamino)-pyridine (DMAP)), ammonia, and a mixture thereof.

From the viewpoint of yield, suppression of by-products, economic efficiency, etc., preferred examples of the base in the step i-b include alkali metal hydroxides, alkali metal carbonates, alkali metal hydrogen carbonates, and a mixture thereof, more preferably alkali metal hydroxides, alkali metal carbonates, and a mixture thereof, and further preferably alkali metal hydroxides.

From the same viewpoint as described above, preferred specific examples of the base in the step i-b include lithium hydroxide, sodium hydroxide, potassium hydroxide, lithium carbonate, sodium carbonate, potassium carbonate, lithium hydrogen carbonate, sodium hydrogen carbonate, potassium hydrogen carbonate and a mixture thereof, more preferably lithium hydroxide, sodium hydroxide, potassium hydroxide, lithium carbonate, sodium carbonate, potassium carbonate and a mixture thereof, still more preferably sodium hydroxide, potassium hydroxide, sodium carbonate, potassium carbonate and a mixture thereof, further preferably sodium hydroxide, potassium hydroxide and a mixture thereof, and particularly preferably sodium hydroxide.

The base in the step i-b may be used singly or in a combination of two or more kinds thereof in any ratio. The base in the step i-b may be in any form as long as the reaction proceeds. Examples of the form of the base in the step i-b include a base-only solid and an aqueous solution with any concentration. Specific examples of the form of the base include, but are not limited to, a flake, a pellet, a bead, a powder and a 10 to 50% aqueous solution, and preferably a flake, a pellet, a bead, and a powder. The form of the base in the step i-b can be appropriately selected by a person skilled in the art.

The amount of the base used in the step i-b may be any amount as long as the reaction proceeds. The amount of the base used in the step i-b may be appropriately adjusted by a person skilled in the art. However, from the viewpoint of yield, suppression of by-products, economic efficiency, etc., the amount of the base used in the step i-b is, for example, 1 to 10 mol, preferably 1 to 8 mol, more preferably 2 to 6 mol, further preferably 3 to 5 mol, and still more preferably 3 to 4 mol based on 1 mol of the compound of the formula (4) (raw material).

(Reaction Solvent in Step i-b)

From the viewpoint of allowing the reaction to smoothly proceed, the reaction in the step i-b is preferably performed in the presence of a solvent.

The solvent in the reaction in the step i-b may be any solvent as long as the reaction proceeds.

In one embodiment, examples of the solvent in the reaction in the step i-b include, but are not limited to, the following: Any combination thereof in any ratio.

In another embodiment, examples of the solvent in the reaction in the step i-b include, but are not limited to, the following:
aromatic hydrocarbon derivatives (e.g., benzene, toluene, xylenes, chlorobenzene, dichlorobenzenes, trichlorobenzenes and nitrobenzene), halogenated aliphatic hydrocarbons (e.g., dichloromethane and 1,2-dichloroethane (EDC)), alcohols (e.g., methanol, ethanol, propanol, 2-propanol, butanol, sec-butanol, isobutanol and tert-butanol (tert-butanol being also referred to as tert-butyl alcohol), pentanol, sec-amyl alcohol, 3-pentanol, 2-methyl-1-butanol, isoamyl alcohol, tert-amyl alcohol, hexanol and cyclohexanol), nitriles (e.g., acetonitrile and propionitrile), carboxylic acid esters (e.g., methyl acetate, ethyl acetate, propyl acetate, isopropyl acetate, butyl acetate and isomers thereof, and pentyl acetate and isomers thereof), ethers (e.g., tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME) and diglyme), ketones (e.g., acetone, methyl ethyl ketone (MEK), methyl isopropyl ketone (MIPK) and methyl isobutyl ketone (MIBK)), amides (e.g., N,N-dimethylformamide (DMF), N,N-dimethylacetamide (DMAC) and N-methylpyrrolidone (NMP)), ureas (e.g., N,N′-dimethylimidazolidinone (DMI) and tetramethylurea), sulfoxides (e.g., dimethyl sulfoxide (DMSO)), sulfones (e.g., sulfolane), water, and any combination thereof in any ratio. “2-Propanol” is also referred to as “isopropyl alcohol” or “isopropanol”.

However, from the viewpoint of yield, suppression of by-products, economic efficiency, etc., preferred examples of the solvent in the reaction in the step i-b include the following: combinations in any ratio of one or more (preferably one or two, more preferably one) selected from aromatic hydrocarbon derivatives, halogenated aliphatic hydrocarbons, alcohols, nitriles, carboxylic acid esters, ethers, ketones, amides, ureas, sulfoxides, and sulfones and water.

More preferred examples of the solvent in the reaction in the step i-b include combinations in any ratio of one or more (preferably one or two, more preferably one) selected from alcohols, nitriles, carboxylic acid esters, ethers, amides, sulfones and water.

More preferred examples of the solvent in the reaction in the step i-b include combinations in any ratio of one or more (preferably one or two, more preferably one) selected from nitriles, carboxylic acid esters, ethers, amides and sulfoxides.

More preferred examples of the solvent in the reaction in the step i-b include combinations in any ratio of one or more (preferably one or two, more preferably one) selected from nitriles, carboxylic acid esters, amides and sulfoxides.

Still more preferred examples of the solvent in the reaction in the step i-b include combinations in any ratio of one or more (preferably one or two, more preferably one) selected from nitriles and amides.

In one embodiment, particularly preferred examples of the solvent in the reaction in the step i-b include nitriles.

From the same viewpoint as described above, preferred specific examples of the solvent in the reaction in the step i-b include combinations in any ratio of one or more (preferably one or two, more preferably one) selected from toluene, xylenes, chlorobenzene, dichlorobenzenes, dichloromethane, 1,2-dichloroethane, methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane.

From the same viewpoint as described above, more preferred specific examples of the solvent in the reaction in the step i-b include combinations in any ratio of one or more (preferably one or two, more preferably one) selected from toluene, xylenes, chlorobenzene, dichlorobenzenes, dichloromethane, 1,2-dichloroethane, methanol, ethanol, propanol, 2-propanol, butanol, sec-butanol, isobutanol, tert-butanol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane.

From the same viewpoint as described above, still more preferred specific examples of the solvent in the reaction in the step i-b include combinations in any ratio of one or more (preferably one or two, more preferably one) selected from toluene, xylenes, chlorobenzene, dichlorobenzenes, dichloromethane, 1,2-dichloroethane, methanol, ethanol, 2-propanol, butanol, tert-butanol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane.

More preferred specific examples of the solvent in the reaction in the step i-b include combinations in any ratio of one or more (preferably one or two, and more preferably one) selected from acetonitrile, ethyl acetate, propyl acetate, isopropyl acetate, butyl acetate, N,N-dimethylformamide (DMF), N,N-dimethylacetamide (DMAC), N-methylpyrrolidone (NMP), dimethyl sulfoxide (DMSO), and isomers thereof.

Still more preferred specific examples of the solvent in the reaction in the step i-b include combinations in any ratio of one or more (preferably one or two, and more preferably one) selected from acetonitrile, N,N-dimethylformamide (DMF), N,N-dimethylacetamide (DMAC), N-methylpyrrolidone (NMP), and dimethyl sulfoxide (DMSO).

Still more preferred specific examples of the solvent in the reaction in the step i-b include combinations in any ratio of one or two (preferably one) selected from acetonitrile, N,N-dimethylformamide (DMF), N,N-dimethylacetamide (DMAC), and N-methylpyrrolidone (NMP).

In one embodiment, a particularly preferred specific example of the solvent in the reaction in the step i-b includes acetonitrile solvent.

The amount of the solvent used in the reaction in the step i-b will be now described. The amount of the solvent used in the reaction in the step i-b is not particularly limited as long as the reaction system can be sufficiently stirred. However, from the viewpoint of yield, suppression of by-products, economic efficiency, etc., in one embodiment, the total amount of the solvent used in the reaction in the step i-b is, for example, 0 (zero) to 5 L (liters), preferably 0.4 to 2.0 L, more preferably 0.5 to 1.5 L, and still more preferably 0.6 to 1.0 L based on 1 mol of the compound of the formula (4) (raw material). In another embodiment, the amount of the organic solvent used in the reaction in the step i-b is, for example, 0.1 to 5 L (liters), preferably 0.3 to 2.0 L, more preferably 0.5 to 1.5 L, further preferably 0.7 to 1.3 L, and still more preferably 0.8 to 1.2 L based on 1 mol of the compound of the formula (4) (raw material).

When a combination of two or more organic solvents is used, the ratio of the two or more organic solvents may be any ratio as long as the reaction proceeds.

(Reaction Temperature in Step i-b)

The reaction temperature in the step i-b is not particularly limited. However, from the viewpoint of yield, suppression of by-products, economic efficiency, etc., the reaction temperature in the step i-b is, for example, −10 (minus 10)° C. to 100° C., preferably −10° C. to 70° C., more preferably −10° C. to 50° C., still more preferably 0 (zero)° C. to 40° C., further preferably 0° C. to 30° C., and further preferably 0° C. to 25° C.

(Reaction Time in Step i-b)

The reaction time in the step i-b is not particularly limited. However, from the viewpoint of yield, suppression of by-products, economic efficiency, etc., in one embodiment, the reaction time in the step i-b is, for example, 1 hour to 48 hours, preferably 1 hour to 24 hours, more preferably 1 hour to 18 hours, and still more preferably 1 hour to 12 hours.

(Adding Method in Step i-b)

The order of adding the compound of the formula (4), the compound of the formula (3), the base, the solvent, etc. is not particularly limited. As long as the reaction proceeds, the addition order thereof may be any order. For example, the base may be added dropwise to a mixture comprising the compound of the formula (4), the compound of the formula (3) and the solvent in a reaction vessel. As another example, the compound of the formula (3) may be introduced to a reaction vessel after adding the compound of the formula (4), the base and the solvent thereto. As still another example, the compound of the formula (3) and the compound of the formula (4) may be successively introduced to a reaction vessel after adding the base and the solvent thereto.

(Step i-c)

The step i-c will now be described.

The step i-c is a step of producing the compound of the formula (7) by reacting a compound of the formula (5) with a compound of the formula (6) in the presence of a base:

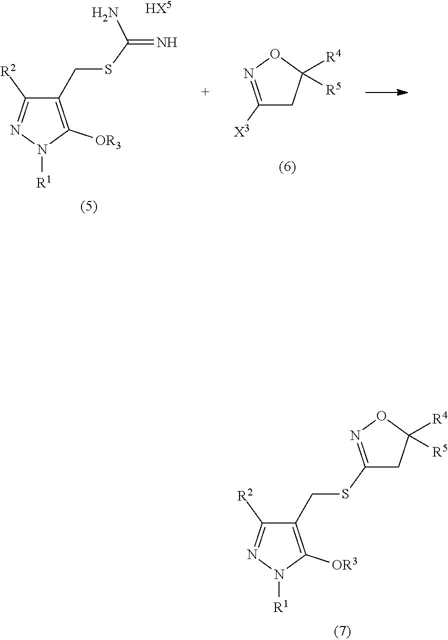

wherein in the formula (5), the formula (6), and the formula (7), R¹, R², R³, R⁴, R⁵, and X³ are as defined above, and X³ is an atom or atomic group forming an acid.

(Raw Material in Step i-c: Compound of Formula (5))

A compound of the formula (5) is used as a raw material in the step i-c. The compound of the formula (5) may be a known compound or may be produced from a known compound according to a known process. For example, the preparation of the compound of the formula (5) is described in Example 15 of WO 2004/013106 A1 (Patent Document 2), which is as

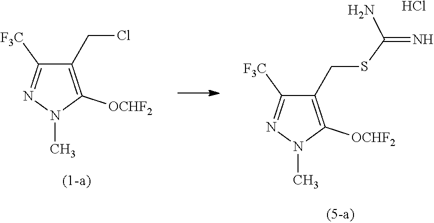

In the formula (5), R¹, R², R³ and X⁵ are as defined above. In the formula (5), examples, preferred examples, more preferred examples, and particularly preferred examples of R¹, R², and R³ are as described above, and examples, preferred examples, more preferred examples and particularly preferred examples of X⁵ are the same as those of X².

A particularly preferred specific example of the compound of the formula (5) is as follows:

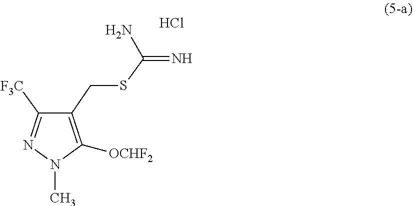 [SMILES: Cl ; Cn1nc(C(F)(F)F)c(CSC(=N)N)c1OC(F)F]

In the reaction in the step i-c, it was presumed that the isothiouronium group in the compound of the formula (5) produces the corresponding thiol group and/or a salt thereof (e.g., generally —S⁻Na⁺ or —S⁻K⁺) and/or an analog thereof. Compounds having thiol groups and/or salts thereof, and/or analogs thereof corresponding to the compound of the formula (5) are equivalents of the compound of the formula (5), and processes using these equivalents are within the scope of the present invention as defined by the appended claims.

(Raw Material in Step i-c: Compound of Formula (6)) A compound of the formula (6) is used as a raw material in the step i-c. The compound of the formula (6) may be a known compound or may be produced from a known compound according to a known process.

X³ in the formula (6) is a leaving group. X³ in the formula (6) may be any atom or atomic group as long as it functions as a leaving group in the reaction in the step 1-c.

From the viewpoint of yield, availability, price, etc., preferred examples of X³ in the formula (6) include halogen atoms, (C1-C4)alkylsulfonyloxy, (C1-C4)haloalkylsulfonyloxy, (C1-C4)alkyl, and benzenesulfonyloxy optionally having a halogen atom, more preferably a chlorine atom, a bromine atom, an iodine atom, methanesulfonyloxy, ethanesulfonyloxy, trifluoromethanesulfonyloxy, benzenesulfonyloxy, p-toluenesulfonyloxy and p-chlorobenzenesulfonyloxy, and particularly preferably a chlorine atom and a bromine atom.

In the formula (6), R⁴, R⁵, and X³ are as defined above. In the formula (6), examples, preferred examples, more preferred examples, and particularly preferred examples of R⁴, R⁵, and X³ are as described above.

Particularly preferred specific examples of the compound of the formula (6) are as follows:

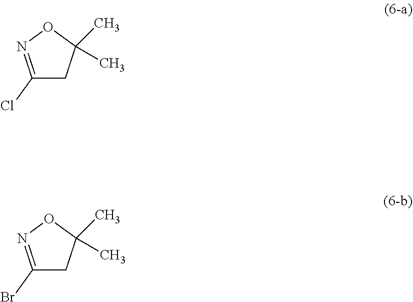

(Raw Material in Step i-c: Amount of Compound of Formula (5) Used)

The amount of the formula (5) used in the step i-c may be any amount as long as the reaction proceeds. The amount of the formula (5) used in the step i-c may be appropriately adjusted by a person skilled in the art. However, from the viewpoint of yield, suppression of by-products, economic efficiency, etc., the amount of the compound of the formula (5) used in the step i-c is, for example, 0.5 to 2.0 mol or more, preferably 0.8 to 1.5 mol, more preferably 1.0 to 1.5 mol, and still more preferably 1.0 to 1.1 mol, based on 1 mol of the compound of the formula (5) (raw material).

(Product in Step i-c: Compound of Formula (7))

The product in the step i-c is a compound of the formula (7) corresponding to the compound of the formula (5) and the compound of the formula (6) used as raw materials.

In the formula (7), examples of R¹, R², R³, R⁴, and R⁵ are as described above.

(Base in Step i-c)

The reaction in the step i-c is performed in the presence of a base. The base may be any base as long as the reaction proceeds. Examples of the base in the step i-c include, but are not limited to, the following:
alkali metal hydroxides (e.g., lithium hydroxide, sodium hydroxide and potassium hydroxide), alkaline earth metal hydroxides (e.g., magnesium hydroxide, calcium hydroxide and barium hydroxide), alkali metal carbonates (e.g., lithium carbonate, sodium carbonate, potassium carbonate and cesium carbonate), alkaline earth metal carbonates (e.g., magnesium carbonate and calcium carbonate), alkali metal hydrogen carbonates (e.g., lithium hydrogen carbonate, sodium hydrogen carbonate and potassium hydrogen carbonate), alkaline earth metal hydrogen carbonates (e.g., calcium hydrogen carbonate), phosphate salts (e.g., sodium phosphate, potassium phosphate and calcium phosphate), hydrogen phosphate salts (e.g., sodium hydrogen phosphate, potassium hydrogen phosphate and calcium hydrogen phosphate), amines (e.g., triethylamine, tributylamine, diisopropylethylamine, 1,8-diazabicyclo[5.4.0]-7-undec-7-ene (DBU), 1,4-diazabicyclo[2.2.2]octane (DABCO), pyridine and 4-(dimethylamino)-pyridine (DMAP)), ammonia, and a mixture thereof.

From the viewpoint of yield, suppression of by-products, economic efficiency, etc., preferred examples of the base in the step i-c include alkali metal hydroxides, alkali metal carbonates, alkali metal hydrogen carbonates, and a mixture thereof, more preferably alkali metal hydroxides, alkali metal carbonates, and a mixture thereof, and further preferably alkali metal hydroxides.

From the same viewpoint as described above, preferred specific examples of the base in the step i-c include lithium hydroxide, sodium hydroxide, potassium hydroxide, lithium carbonate, sodium carbonate, potassium carbonate, lithium hydrogen carbonate, sodium hydrogen carbonate, potassium hydrogen carbonate and a mixture thereof, more preferably lithium hydroxide, sodium hydroxide, potassium hydroxide, lithium carbonate, sodium carbonate, potassium carbonate and a mixture thereof, still more preferably sodium hydroxide, potassium hydroxide, sodium carbonate, potassium carbonate and a mixture thereof, further preferably sodium hydroxide, potassium hydroxide and a mixture thereof, and particularly preferably sodium hydroxide.

The base in the step i-c may be used singly or in a combination of two or more kinds thereof in any ratio. The base in the step i-c may be in any form as long as the reaction proceeds. Examples of the form of the base in the step i-c include a base-only solid and an aqueous solution with any concentration. Specific examples of the form of the base include, but are not limited to, a flake, a pellet, a bead, a powder and a 10 to 50% aqueous solution, and preferably a 20 to 50% aqueous solution (e.g., a 25% aqueous sodium hydroxide solution and a 48% aqueous sodium hydroxide solution, preferably a 48% aqueous sodium hydroxide solution). The form of the base in the step i-c can be appropriately selected by a person skilled in the art.

The amount of the base used in the step i-c may be any amount as long as the reaction proceeds. The amount of the base used in the step i-c can be appropriately adjusted by a person skilled in the art. However, from the viewpoint of yield, suppression of by-products, economic efficiency, etc., in one embodiment, the amount of the base used in the step i-c is, for example, 5 to 10 mol, preferably 5 to 8 mol, more preferably 5 to 7 mol, and still more preferably 5 to 6 mol, based on 1 mol of the compound of the formula (6) (raw material). In another embodiment, for example, the amount is 1 to 15 mol, preferably 1 to 10 mol, more preferably 2 to 9 mol, still more preferably 4 to 8 mol, and further preferably 5 to 6 mol, based on 1 mol of the compound of the formula (6) (raw material).

(Reaction Solvent in Step i-c)

From the viewpoint of allowing the reaction to smoothly proceed, the reaction in the step i-c is preferably performed in the presence of a solvent. The solvent in the reaction in the step i-c may be any solvent as long as the reaction proceeds.

Examples of the solvent in the reaction in the step i-c include, but are not limited to, the following:
aromatic hydrocarbon derivatives (e.g., benzene, toluene, xylenes, chlorobenzene, dichlorobenzenes, trichlorobenzenes and nitrobenzene), halogenated aliphatic hydrocarbons (e.g., dichloromethane and 1,2-dichloroethane (EDC)), alcohols (e.g., methanol, ethanol, propanol, 2-propanol, butanol, sec-butanol, isobutanol and tert-butanol (tert-butanol being also referred to as tert-butyl alcohol), pentanol, sec-amyl alcohol, 3-pentanol, 2-methyl-1-butanol, isoamyl alcohol, tert-amyl alcohol, hexanol and cyclohexanol), nitriles (e.g., acetonitrile and propionitrile), carboxylic acid esters (e.g., methyl acetate, ethyl acetate, propyl acetate, isopropyl acetate, butyl acetate and isomers thereof, and pentyl acetate and isomers thereof), ethers (e.g., tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME) and diglyme), ketones (e.g., acetone, methyl ethyl ketone (MEK), methyl isopropyl ketone (MIPK) and methyl isobutyl ketone (MIBK)), amides (e.g., N,N-dimethylformamide (DMF), N,N-dimethylacetamide (DMAC) and N-methylpyrrolidone (NMP)), ureas (e.g., N,N′-dimethylimidazolidinone (DMI) and tetramethylurea), sulfoxides (e.g., dimethyl sulfoxide (DMSO)), sulfones (e.g., sulfolane), water, and any combination thereof in any ratio. “2-Propanol” is also referred to as “isopropyl alcohol” or “isopropanol”.

However, from the viewpoint of yield, suppression of by-products, economic efficiency, etc., preferred examples of the solvent in the reaction in the step i-c include the following: combinations of one or more (preferably one or two, more preferably one) organic solvents selected from aromatic hydrocarbon derivatives, halogenated aliphatic hydrocarbons, alcohols, nitriles, carboxylic acid esters, ethers, ketones, amides, ureas, sulfoxides, and sulfones, with a water solvent in any ratio.

More preferred examples of the solvent in the reaction in the step i-c include combinations of one or more (preferably one or two, more preferably one) organic solvents selected from alcohols, nitriles, carboxylic acid esters, ethers, amides and sulfones with a water solvent in any ratio.

More preferred examples of the solvent in the reaction in the step i-c include combinations of one or more (preferably one or two, more preferably one) organic solvents selected from alcohols, nitriles, carboxylic acid esters, ethers and amides with a water solvent in any ratio.

More preferred examples of the solvent in the reaction in the step i-c include combinations of one or more (preferably one or two, more preferably one) organic solvents selected from alcohols, nitriles, carboxylic acid esters and amides with a water solvent in any ratio.

More preferred examples of the solvent in the reaction in the step i-c include combinations of one or more (preferably one or two, more preferably one) organic solvents selected from alcohols, nitriles and carboxylic acid esters with a water solvent in any ratio.

Still more preferred examples of the solvent in the reaction in the step i-c include combinations of one or more (preferably one or two, more preferably one) organic solvents selected from nitriles and carboxylic acid esters with a water solvent in any ratio.

In one embodiment, particularly preferred examples of the solvent in the reaction in the step i-c include combinations of nitriles with a water solvent in any ratio.

In another embodiment, particularly preferred examples of the solvent in the reaction in the step i-c include combinations of carboxylic acid esters with a water solvent in any ratio.

From the same viewpoint as described above, preferred specific examples of the solvent in the reaction in the step i-c include combinations of one or more (preferably one or two, more preferably one) organic solvents selected from toluene, xylenes, chlorobenzene, dichlorobenzenes, dichloromethane, 1,2-dichloroethane, methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane with a water solvent in any ratio.

From the same viewpoint as described above, more preferred specific examples of the solvent in the reaction in the step i-c include combinations of one or more (preferably one or two, more preferably one) organic solvents selected from toluene, xylenes, chlorobenzene, dichlorobenzenes, dichloromethane, 1,2-dichloroethane, methanol, ethanol, propanol, 2-propanol, butanol, sec-butanol, isobutanol, tert-butanol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane with a water solvent in any ratio.

From the same viewpoint as described above, still more preferred specific examples of the solvent in the reaction in the step i-c include combinations of one or more (preferably one or two, more preferably one) organic solvents selected from toluene, xylenes, chlorobenzene, dichlorobenzenes, dichloromethane, 1,2-dichloroethane, methanol, ethanol, 2-propanol, butanol, tert-butanol, acetonitrile, ethyl acetate, propyl acetate, isopropyl acetate, butyl acetate and isomers thereof (in the present invention, the “isomer of butyl acetate” being an equivalent of “butyl acetate”), tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), diglyme, acetone, methyl ethyl ketone (MEK), methyl isopropyl ketone (MIPK), methyl isobutyl ketone (MIBK), N,N-dimethylformamide (DMF), N,N-dimethylacetamide (DMAC), N-methylpyrrolidone (NMP), N,N′-dimethylimidazolidinone (DMI), tetramethylurea, dimethyl sulfoxide (DMSO) and sulfolane with a water solvent in any ratio.

More preferred specific examples of the solvent in the reaction in the step i-c include combinations of one or more (preferably one or two, more preferably one) organic solvents selected from methanol, ethanol, 2-propanol, butanol, tert-butanol, acetonitrile, ethyl acetate, propyl acetate, isopropyl acetate, butyl acetate and isomers thereof with a water solvent in any ratio.

Still more preferred specific examples of the solvent in the reaction in the step i-c include combinations of one or more (preferably one or two, more preferably one) organic solvents selected from butanol, acetonitrile, ethyl acetate, propyl acetate, isopropyl acetate and butyl acetate with a water solvent in any ratio.

Further preferred specific examples of the solvent in the reaction in the step i-c include combinations of one or more (preferably one or two, more preferably one) organic solvents selected from acetonitrile, ethyl acetate, isopropyl acetate and butyl acetate with a water solvent in any ratio.

Still further preferred specific examples of the solvent in the reaction in the step i-c include combinations of one or two (preferably one) organic solvents selected from acetonitrile and butyl acetate with a water solvent in any ratio.

In one embodiment, a particularly preferred specific example of the solvent in the reaction in the step i-c includes a combination of an acetonitrile solvent with a water solvent in any ratio.

In another embodiment, a particularly preferred specific example of the solvent in the reaction in the step i-c includes a combination of a butyl acetate solvent with a water solvent in any ratio.

In either case, the solvent may be in a single layer or may be separated into two layers as long as the reaction proceeds.

The amount of the solvent used in the reaction in the step i-c will now be described. The “total amount of the solvent used in the reaction” is the sum total of the amounts of all the organic solvents and the amount of the water solvent used in the reaction. The organic solvent and the water solvent used in the working-up (e.g., isolation and purification) after the reaction are not included. The “organic solvent” used in the reaction includes the organic solvent in the raw material solution and that in the reactant solution. The “water solvent” used in the reaction includes the water in the raw material solution and that in the reactant solution (e.g., water in a 48% aqueous sodium hydroxide solution).

The total amount of the solvent used in the reaction in the step i-c is not particularly limited as long as the reaction system can be sufficiently stirred. However, from the viewpoint of yield, suppression of by-products, economic efficiency, etc., in one embodiment, the total amount of the solvent used in the reaction in the step i-c is, for example, 0.1 to 10 L (liters), preferably 0.5 to 5 L, more preferably 1 to 5 L, still more preferably 1 to 3 L, and further preferably 1 to 2 L, based on 1 mol of the compound of the formula (6) (raw material). In another embodiment, the total amount of the solvent used in the reaction in the step i-c is, for example, 1.5 to 3.0 L (liters), preferably 1.5 to 2.5 L, and more preferably 1.5 to 2.0 L, based on 1 mol of the compound of the formula (6) (raw material). In still another embodiment, the total amount of the solvent used in the reaction in the step i-c is, for example, 1.7 to 3.0 L (liters), preferably 1.7 to 2.5 L, and more preferably 1.7 to 2.0 L, based on 1 mol of the compound of the formula (6) (raw material).

From the same viewpoint as described above, in one embodiment, the amount of the organic solvent used in the reaction in the step i-c is, for example, 0 (zero) to 5 L (liters), preferably 0.4 to 2.0 L, more preferably 0.5 to 1.5 L, still more preferably 0.6 to 1.0 L, and further preferably 0.7 to 0.9 L, based on 1 mol of the compound of the formula (6) (raw material). In another embodiment, the amount of the organic solvent used in the reaction in the step i-c is, for example, 0.1 to 5 L (liters), preferably 0.3 to 2.0 L, more preferably 0.4 to 1.5 L, still more preferably 0.5 to 1.0 L, and further preferably 0.6 to 0.8 L, based on 1 mol of the compound of the formula (6) (raw material).

From the same viewpoint as described above, the amount of the water solvent used in the reaction in the step i-c is, for example, 0.1 to 5 L (liters), preferably 0.5 to 2.0 L, more preferably 0.5 to 1.5 L, still more preferably 0.7 to 1.4 L, and further preferably 0.9 to 1.2 L, based on 1 mol of the compound of the formula (6) (raw material).

When a combination of two or more organic solvents is used, the ratio of the two or more organic solvents may be any ratio as long as the reaction proceeds.

When a combination of an organic solvent and a water solvent is used, the ratio of the organic solvent and the water solvent may be any ratio as long as the reaction proceeds.

(Reaction Temperature in Step i-c)

The reaction temperature in the step i-c is not particularly limited. However, from the viewpoint of yield, suppression of by-products, economic efficiency, etc., the reaction temperature in the step i-c is, for example, −10 (minus 10)° C. to 100° C., preferably −10° C. to 70° C., more preferably −10° C. to 50° C., still more preferably 0 (zero)° C. to 40° C., further preferably 0° C. to 30° C., and further preferably 0° C. to 25° C.

(Reaction Time in Step i-c)

The reaction time in the step i-c is not particularly limited. However, from the viewpoint of yield, suppression of by-products, economic efficiency, etc., in one embodiment, the reaction time in the step i-c is, for example, 4 hours to 48 hours, preferably 4 hours to 24 hours, more preferably 4 hours to 18 hours, and still more preferably 4 hours to 12 hours. In another embodiment, the reaction time in the step i-c is, for example, 1 hour to 48 hours, preferably 1 hour to 24 hours, more preferably 3 hours to 18 hours, and still more preferably 3 hours to 12 hours. However, the reaction time can be adjusted appropriately by a person skilled in the art.

(Adding Method in Step i-c)

The order of adding the compound of the formula (5), the compound of the formula (6), the base, the solvent, etc. is not particularly limited. As long as the reaction proceeds, the addition order thereof may be any order. For example, the base may be added dropwise to a mixture comprising the compound of the formula (5), the compound of the formula (6) and the solvent in a reaction vessel. As another example, the compound of the formula (5) may be added dropwise to a reaction vessel after adding the compound of the formula (6), the base and the solvent thereto. As still another example, the compound of the formula (5) and the compound of the formula (6) may be successively added dropwise to a reaction vessel after adding the base and the solvent thereto.

(Working-up in Step i-c, Isolation and/or Purification)

The compound of the formula (7), especially the compound (7-a), which is the product in the step i-c, can be used as a raw material in the step ii. The compound of the general formula (7) obtained in the step i-c may be isolated and/or purified and then used in the next step, or may be used in the next step without being isolated. Whether or not to perform the working-up (isolation and/or purification) can be appropriately determined by a person skilled in the art according to the purpose and situation.

The compounds of the formula (7), especially the compound (7-a), which is the target product in the step 1-c, can be isolated and purified from the reaction mixture by any of methods known to a person skilled in the art (e.g., extraction, washing, crystallization including recrystallization, crystal washing and/or other procedures) and improved methods thereof, and any combination thereof.

In the working-up step (isolation and/or purification), the following procedures may be performed, but are not limited thereto: in the working-up, an extraction procedure and a washing procedure which include separation of an organic layer and an aqueous layer may be performed. When the mixture is separated into an organic layer and an aqueous layer, the mixture may be separated while being hot. For example, when separating the organic layer from the aqueous layer, a hot mixture may be used, or the mixture may be heated. Impurities may be removed by a filtration procedure including hot filtration.

In the washing procedure, if possible, the product dissolved or suspended in an organic solvent may be washed with water, hot water, an aqueous alkaline solution (e.g., a 5% to saturated aqueous sodium hydrogen carbonate solution or a 1 to 10% aqueous sodium hydroxide solution), or an acidic aqueous solution (e.g., 5 to 35% hydrochloric acid or 5 to 35% sulfuric acid). Such washing procedures may be combined.

When performing crystallization of the product including recrystallization and washing of crystals, the description in the step ii described later may be referred to.

In any of the above procedures, the temperature can be appropriately adjusted by a person skilled in the art according to the purpose and situation.

In any procedure of the working-up and the procedure of using the product in the next step, the amount of a solvent can be appropriately adjusted by a person skilled in the art by addition and removal thereof. Furthermore, recovery and recycle of the solvent may be optionally performed. For example, the recovery and recycle of the solvent used in the reaction may be performed, and the recovery and recycle of the solvent used in the working-up (isolation and/or purification) may be performed.

Working-up (isolation and/or purification) can be performed by appropriately combining all or some of the procedures described above. Optionally, the above procedure may be repeated according to the purpose. In addition, a person skilled in the art can appropriately select a combination of any of the above procedures and their order.

(Step ii (Oxidation Reaction))

The step ii will now be described.

The step ii is an oxidation reaction. In the step ii, a compound of the formula (8) is produced from the compound of the formula (7) by oxidation.

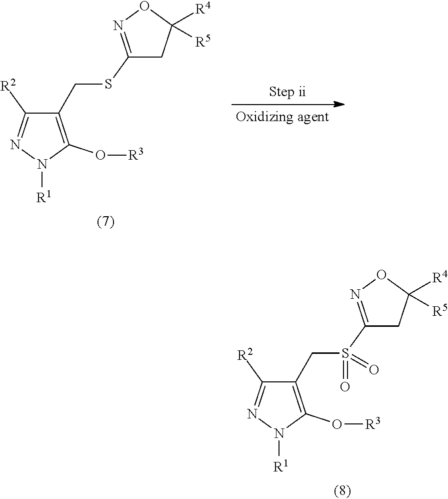

wherein in the formula (7) and the formula (8), R¹, R², R³, R⁴, and R⁵ are as defined above.

Examples of the oxidation reaction in the step ii include a method using an oxidizing agent such as hydrogen peroxide, hypochlorite, or peroxide, and dimethyl sulfoxide oxidation such as ozone oxidation, or Swern oxidation. Performing the reaction in the step ii using a hypochlorite such as sodium hypochlorite or potassium hypochlorite, sodium hydrogen persulfate, sodium persulfate (sodium peroxodisulfate), potassium persulfate, ammonium persulfate, potassium hydrogen persulfate (a peroxide such as peroxymonosulfate or Oxone (registered trademark)), or the like in place of hydrogen peroxide is an equivalent of the present invention and is within the scope of the present invention.

The step ii is preferably a step of producing the compound of the formula (8) by reacting the compound of the formula (7) with hydrogen peroxide under specific conditions:

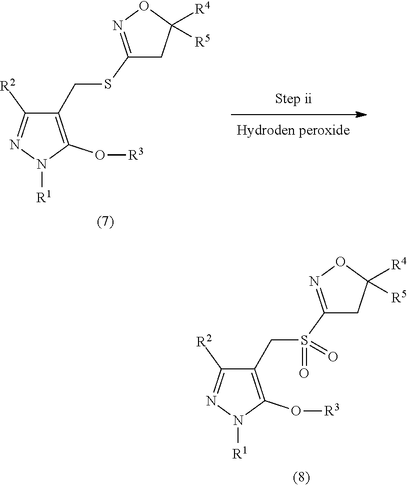

wherein in the formula (7) and the formula (8), R¹, R², R³, R⁴, and R⁵ are as defined above.

(Raw Material in Step ii; Compound of Formula (7))

A compound of the formula (7) is used as a raw material in the step ii. The compound of the formula (7) may be a known compound or may be produced from a known compound according to a known process. For example, the preparation of the compound of the formula (7) is described in WO 2004/013106 A1 (Patent Document 2), Reference Examples 1-1, 1-2 and 1-3, WO 2005/105755 A1 (Patent Document 3), Examples 3 to 5 and WO 2005/095352 A1 (Patent Document 4), Examples 1 to 5. In addition, the preparation of the compound of the formula (7) can be performed by a similar method. However, it is preferred that the compound of the formula (7) is produced by the process of the present invention. That is, the compound of the formula (7) is preferably produced by the process comprising the steps i-a, i-b, and i-c described herein.

(Product in Step ii; Compound of Formula (8))

The product in the step ii is a compound of the formula (8) corresponding to the compound of the formula (7) used as a raw material.

In the formula (7) and the formula (8), R¹, R², R³, R⁴, and R⁵ are as defined above. In the formula (7) and the formula (8), examples, preferred examples, more preferred examples, and particularly preferred examples of R¹, R², R³, R⁴, and R⁵ are as described above. It has been expected that a desired oxidation reaction is difficult to proceed in using the compound of the formula (7), particularly, in using the compounds having these preferable, more preferable, or particularly preferable substituents. Contrary to the expectation, however, it has been found that the oxidation reaction sufficiently proceeds under the reaction conditions of the present invention.

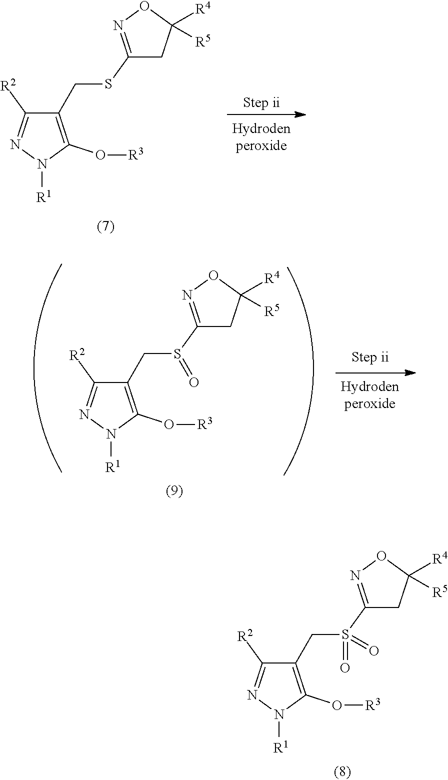

wherein in the formula (7), the formula (8), and the formula (9), R¹, R², R³, R⁴, and R⁵ are as defined above.

After obtaining the formula (9) by oxidizing the formula (7), the resultant may be oxidized to the formula (8).

A particularly preferred specific example of the compound of the formula (8) is as follows:

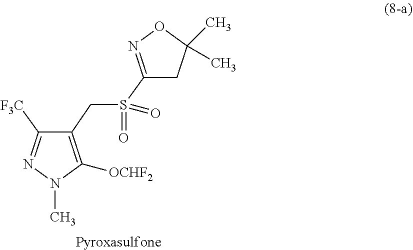

As described above, in the process of producing the compound of the formula (8) (SO₂ derivative) from the compound of the formula (7) (S derivative), it is desired that the oxidation reaction sufficiently proceeds and the proportion of the compound of the formula (9) (SO derivative) in the product is sufficiently low. For example, in the reaction mixture after the reaction in the step ii, the ratio of the compound of the formula (9) (SO derivative) is preferably 10% or less, more preferably 5% or less, still more preferably 3% or less, further preferably 2% or less, and further preferably 1% or less.

(Oxidizing Agent in Step ii)

In the reaction in the step ii, the hypochlorite, alkali metal persulfate, an ammonium persulfate salt, alkali metal hydrogen persulfate, peroxide, etc. described above can be used as the oxidizing agent. In one embodiment, hydrogen peroxide, an alkali metal persulfate, an ammonium persulfate salt, and an alkali metal hydrogen persulfate are preferably used, hydrogen peroxide and an alkali metal hydrogen persulfate are more preferably used, and hydrogen peroxide, sodium hydrogen persulfate, sodium persulfate, potassium persulfate, ammonium persulfate, and potassium hydrogen persulfate are further preferably used. In another embodiment, hydrogen peroxide is preferably used. In still another embodiment, sodium hydrogen persulfate, sodium persulfate, potassium persulfate, ammonium persulfate, and potassium hydrogen persulfate are preferably used, and potassium hydrogen persulfate is more preferably used.

The form of the hydrogen peroxide in the step ii may be any form as long as the reaction proceeds. The form of the hydrogen peroxide in the step ii can be suitably selected by a person skilled in the art. In view of safety, danger, economic efficiency, etc., however, preferred examples of the form of the hydrogen peroxide include a 10 to 70 wt % aqueous hydrogen peroxide solution, more preferably a 20 to 70 wt % aqueous hydrogen peroxide solution, still more preferably a 25 to 65 wt % aqueous hydrogen peroxide solution, further preferably a 30 to 65 wt % aqueous hydrogen peroxide solution, and particularly preferably a 30 to 60 wt % aqueous hydrogen peroxide solution. Specific examples of the form of the hydrogen peroxide include, but are not limited to, a 25 wt % aqueous hydrogen peroxide solution, a 30 wt % aqueous hydrogen peroxide solution, a 35 wt % aqueous hydrogen peroxide solution, a 50 wt % aqueous hydrogen peroxide solution and a 60 wt % aqueous hydrogen peroxide solution. The range of the concentration of the hydrogen peroxide may be any combination of the lower limits and the upper limits of the above-described ranges, and such combinations of the lower limits and the upper limits of the ranges are within the scope of the present invention.

The amount of the hydrogen peroxide used in the step ii may be any amount as long as the reaction proceeds. The amount of the hydrogen peroxide used in the step ii may be appropriately adjusted by a person skilled in the art. From the viewpoint of yield, suppression of by-products, economic efficiency, safety, etc., however, the lower limit of the amount of the hydrogen peroxide used is, for example, 2 mol or more, 2.3 mol or more, 2.5 mol or more, 2.8 mol or more, or 3 mol or more based on 1 mol of the compound of the formula (7) (raw material). The upper limit of the amount of the hydrogen peroxide used is, for example, 10 mol or less, 8 mol or less, 7 mol or less, 6 mol or less, 5 mol or less, 4 mol or less, or 3 mol or less based on 1 mol of the compound of the formula (7) (raw material). The amount of the hydrogen peroxide used is within a range of any combination of the lower limits and the upper limits of the ranges described above. In one embodiment, the amount of the hydrogen peroxide used in the step ii is, for example, 2 mol or more, preferably 2 to 8 mol, more preferably 2 to 6 mol, further preferably 2 to 5 mol, further preferably 2 to 4 mol, further preferably 2 to 3, and still further preferably 2.3 to 3 mol based on 1 mol of the compound of the formula (7) (raw material). In another embodiment, the amount of the hydrogen peroxide used in the step ii is, for example, 2 mol or more, preferably 2 to 10 mol, more preferably 3 to 6 mol, and further preferably 3 to 5 mol based on 1 mol of the compound of the formula (7) (raw material).

Specific examples of the alkali metal persulfate, ammonium persulfate salt, or alkali metal hydrogen persulfate in the step ii include, but are not limited to, the following: sodium persulfate, potassium persulfate, and ammonium persulfate. Specific examples of the hydrogen persulfate in the step ii include, but are not limited to, the following: sodium hydrogen persulfate, and potassium hydrogen persulfate.

The amount of the alkali metal persulfate, ammonium persulfate salt or alkali metal hydrogen persulfate used in the step ii is any amount as long as the reaction proceeds. The amount of the alkali metal persulfate, ammonium persulfate salt or alkali metal hydrogen persulfate used in the step ii can be appropriately selected by a person skilled in the art. In one embodiment, the amount of the alkali metal persulfate, ammonium persulfate salt or alkali metal hydrogen persulfate used in the step ii is, for example, 1.0 to 2.0 mol, preferably 1.0 to 1.5 mol, and more preferably 1.0 to 1.2 mol based on 1 mol of the compound of the formula (7) (raw material).

(Step ii: In the Absence of Transition Metal)

An oxidation reaction using hydrogen peroxide as an oxidizing agent in the presence of a transition metal catalyst has been reported. In the process of the present invention, however, there is no need for a transition metal catalyst. Accordingly, the term “in the absence of a transition metal” means that a catalyst containing a transition metal catalyst is not used. Accordingly, “in the absence of a transition metal” herein can be optionally replaced by “in the absence of a transition metal catalyst”. Examples of the transition metal not used in the step ii include, but are not limited to, tungsten, molybdenum, iron, manganese, vanadium, niobium, tantalum, titanium, zirconium, and copper. Examples of the transition metal catalyst not used in the step ii include, but are not limited to, tungsten catalysts (e.g., sodium tungstate dihydrate), molybdenum catalysts (e.g., ammonium molybdate tetrahydrate), iron catalysts (e.g., iron (III) acetylacetonate, and iron (III) chloride), manganese catalysts (e.g., manganese (III) acetylacetonate), vanadium catalysts (e.g., vanadyl acetylacetonate), niobium catalysts (e.g., sodium niobate), tantalum catalysts (e.g., lithium tantalate), titanium catalysts (e.g., titanium acetylacetonate, and titanium tetrachloride), zirconium catalysts (e.g., zirconium chloride oxide octahydrate) and copper catalysts (e.g., copper (II) acetate, and copper (I) bromide).

(Acidic Compound in Step ii)

The reaction in the step ii may be performed in the presence of an acidic compound. From the viewpoint of yield, suppression of by-products, economic efficiency, etc., preferred examples of the acidic compound in the step ii include, but are not limited to, the following: mineral acids, carboxylic acids, sulfonic acids, phosphoric acids, and a mixture thereof, and more preferably mineral acids, carboxylic acids, and a mixture thereof. The acidic compound may be a salt or acid anhydride thereof as long as the reaction proceeds. Those forming salts (e.g., sodium salts and potassium salts) and/or anhydrides of the acids (e.g., acetic anhydride, and trifluoroacetic anhydride) are also included. In other words, the term “acidic compound” used herein encompasses salts and acid anhydrides thereof. A process for performing the reaction in the step ii in the presence of a salt and/or an acid anhydride of the acidic compound is within the scope of the present invention as defined by the appended claims. As is understood from Example 2-29 described below, for example, a process using a salt of sulfuric acid (e.g., an alkali metal hydrogen sulfate such as sodium hydrogen sulfate or potassium hydrogen sulfate) as the acidic compound is within the scope of the present invention. In addition, a process using an alkali metal sulfate such as sodium sulfate or potassium sulfate is an equivalent of the present invention, and is within the scope of the present invention.

From the same viewpoint as described above, preferred specific examples of the acidic compound in the step ii include, but are not limited to, the following: mineral acids (e.g., nitric acid, sulfuric acid, sodium hydrogen sulfate, and potassium hydrogen sulfate), carboxylic acids (e.g., formic acid, acetic acid, trifluoroacetic acid, trichloroacetic acid, dichloroacetic acid, monochloroacetic acid, maleic acid, phthalic acid, benzoic acid, acetic anhydride, and trifluoroacetic anhydride), sulfonic acids (e.g., methanesulfonic acid, trifluoromethanesulfonic acid, benzenesulfonic acid, and p-toluenesulfonic acid), and phosphoric acids (e.g., phosphoric acid, methyl phosphate, ethyl phosphate, and phenyl phosphate), more preferably sulfuric acid, sodium hydrogen sulfate, potassium hydrogen sulfate, acetic acid, trifluoroacetic acid, and a mixture thereof, more preferably sulfuric acid, potassium hydrogen sulfate, acetic acid, trifluoroacetic acid, and a mixture thereof, and further preferably sulfuric acid, acetic acid, trifluoroacetic acid, and a mixture thereof.

The concentration of the sulfuric acid can be appropriately selected by a person skilled in the art. The concentration of the sulfuric acid is not particularly limited, and is preferably 10% to 100%, more preferably 30% to 100%, and further preferably 50% to 100%.

The acidic compound in the step ii may be used singly or in a combination of two or more kinds thereof in any ratio. The acidic compound in the step ii may be in any form as long as the reaction proceeds. The form of the acidic compound can be appropriately selected by a person skilled in the art. In addition, immobilized reactants and catalysts are known in general. These are reactants and catalysts immobilized on carriers through adsorption or covalent bond. An immobilized acidic compound is not excluded from the scope of the present invention. On the other hand, in view of availability and reactivity, a non-immobilized acidic compound is preferred. The amount of the acidic compound used in the step ii may be any amount as long as the reaction proceeds. The amount of the acidic compound used may be appropriately adjusted by a person skilled in the art. From the viewpoint of yield, suppression of by-products, economic efficiency, etc., however, the amount of the acidic compound used is, for example, within a range of any combination of the following lower limits and upper limits. In one embodiment, the amount of the acidic compound used is larger than 0 (zero) mol, preferably 0.1 to 100 mol, more preferably 0.5 to 50 mol, further preferably 1 to 40 mol, and still further preferably 2 to 30 mol based on 1 mol of the compound of the formula (7) (raw material). In another embodiment, the amount of the acidic compound used is, for example, larger than 0 (zero) mol, preferably 1 to 100 mol, more preferably 1 to 50 mol, and further preferably 1 to 30 mol based on 1 mol of the compound of the formula (7) (raw material). In another embodiment, for example, when the acidic compound is sulfuric acid, the amount of the acidic compound used is, for example, larger than 0 (zero) mol, preferably 0.2 to 10 mol, more preferably 0.2 to 5 mol, and further preferably 0.2 to 3 mol based on 1 mol of the compound of the formula (7) (raw material). In still another embodiment, when the acidic compound is sulfuric acid, the amount of the acidic compound used is, for example, 0.25 to 4 mol, 0.25 to 3.5 mol, preferably 0.3 to 3.5 mol, and 0.3 to 3 mol based on 1 mol of the compound of the formula (7) (raw material). “When the acidic compound is sulfuric acid” corresponds to, for example, reactions using sulfuric acid described in Examples 2-1 to 2-18.

The acidic compound may be used as a solvent. In this case, the acidic compound contributes to the reaction itself as well as functions as a solvent.

(Base in Step ii)

The reaction in the step ii may be performed in the presence of a base. From the viewpoint of yield, suppression of by-products, economic efficiency, etc., preferred examples of the base in step ii include, but are not limited to, the following: carbonates, hydrogen carbonates, and a mixture thereof, preferably metal hydrogen carbonates, metal carbonates, and a mixture thereof, more preferably alkali metal hydrogen carbonates, alkali metal carbonates, and a mixture thereof, and further preferably alkali metal carbonates.

From the same viewpoint as described above, preferred specific examples of the base in the step ii include, but are not limited to, the following: lithium hydrogen carbonate, sodium hydrogen carbonate, potassium hydrogen carbonate, cesium hydrogen carbonate, magnesium hydrogen carbonate, calcium hydrogen carbonate, lithium carbonate, sodium carbonate, potassium carbonate, cesium carbonate, magnesium carbonate, and calcium carbonate, more preferably sodium hydrogen carbonate, potassium hydrogen carbonate, sodium carbonate, and potassium carbonate, and further preferably potassium carbonate, potassium hydrogen carbonate, and sodium hydrogen carbonate.

The base in the step ii may be used singly or in a combination of two or more kinds thereof in any ratio. The base in the step ii may be in any form as long as the reaction proceeds. The form of the base can be appropriately selected by a person skilled in the art. In addition, immobilized reactants and catalysts are known in general. These are reactants and catalysts immobilized on carriers through adsorption or covalent bond. An immobilized base is not excluded from the scope of the present invention. On the other hand, in view of availability and reactivity, a non-immobilized base is preferred. The amount of the base used in the step ii may be any amount as long as the reaction proceeds. From the viewpoint of yield, suppression of by-products, economic efficiency, etc., the amount of the base used is, for example, within a range of any combination of the following lower limits and upper limits. In one embodiment, the amount of the base used is, for example, 0 (zero) to 2 mol, preferably 0.01 to 1 mol, more preferably 0.05 to 1 mol, and further preferably 0.1 to 0.8 mol based on 1 mol of the compound of the formula (7) (raw material). In another embodiment, the amount of the base used is, for example, 0.05 to 5 mol, preferably 0.1 to 3 mol, and more preferably 0.4 to 1.5 mol based on 1 mol of the compound of the formula (7) (raw material). In another embodiment, the amount of the base used is, for example, 0.4 to 0.6 mol based on 1 mol of the compound of the formula (7) (raw material).

(Nitrile Compound in Step ii)

The reaction in the step ii may be performed in the presence of a nitrile compound. A nitrile compound refers to a compound having a nitrile group. Preferred examples of the nitrile compound in the step ii include, but are not limited to, the following: alkyl nitrile derivatives, benzonitrile derivatives, and a mixture thereof, and more preferably alkyl nitride derivatives and a mixture thereof.

From the same viewpoint as described above, specific preferred examples of the nitrile compound in the step ii include, but are not limited to, the following: acetonitrile, propionitrile, butyronitrile, isobutyronitrile, succinonitrile, benzonitrile, and p-nitrobenzonitrile, preferably acetonitrile, isobutyronitrile, succinonitrile, benzonitrile, and p-nitrobenzonitrile, more preferably acetonitrile, isobutyronitrile, and succinonitrile, and further preferably acetonitrile.

The nitrile compound in the step ii may be used singly or in a combination of two or more kinds thereof in any ratio. The amount of the nitrile compound used in the step ii may be any amount as long as the reaction proceeds. The amount of the nitrile compound used may be appropriately adjusted by a person skilled in the art. From the viewpoint of yield, suppression of by-products, economic efficiency, etc., however, the amount of the nitrile compound used is, larger than 0 (zero) mol, preferably 1 to 100 mol, more preferably 1 to 50 mol, and further preferably 1 to 35 mol based on 1 mol of the compound of the formula (7) (raw material). The nitrile compound may be used as a solvent. In this case, the nitrile compound contributes to the reaction itself as well as functions as a solvent.

(Ketone Compound in Step ii)

The reaction in the step ii may be performed in the presence of or in the absence of a ketone compound. A ketone compound refers to a compound having a ketone group. It can be appropriately determined by a person skilled in the art whether or not a ketone compound is used. Examples of the ketone compound in the step ii include, but are not limited to, the following: 2,2,2-trifluoroacetophenone, methyl isobutyl ketone, and cyclohexanone.

The ketone compound in the step ii may be used singly or in a combination of two or more kinds thereof in any ratio. The amount of the ketone compound used in the step ii may be any amount as long as the reaction proceeds. The amount of the ketone compound used may be appropriately adjusted by a person skilled in the art. From the viewpoint of yield, suppression of by-products, economic efficiency, etc., however, the amount of the ketone compound used is, for example, 0.01 to 1.0, preferably 0.05 to 0.8 mol, and more preferably 0.1 to 0.6 mol based on 1 mol of the compound of the formula (7) (raw material).

(Reaction Solvent in Step ii)

From the viewpoint of allowing the reaction to smoothly proceed, the reaction in the step ii is preferably performed in the presence of a solvent. The solvent in the reaction in the step ii may be any solvent as long as the reaction proceeds.

Examples of the solvent in the reaction in the step ii include, but are not limited to, the following:
aromatic hydrocarbon derivatives (e.g., benzene, toluene, xylenes, chlorobenzene, dichlorobenzenes, trichlorobenzenes and nitrobenzene), halogenated aliphatic hydrocarbons (e.g., dichloromethane and 1,2-dichloroethane (EDC)), alcohols (e.g., methanol, ethanol, propanol, 2-propanol, butanol, sec-butanol, isobutanol and tert-butanol (tert-butanol is also referred to as tert-butyl alcohol), pentanol, sec-amyl alcohol, 3-pentanol, 2-methyl-1-butanol, isoamyl alcohol, tert-amyl alcohol, hexanol and cyclohexanol), nitriles (e.g., acetonitrile and propionitrile,
butyronitrile, isobutyronitrile, succinonitrile, and benzonitrile), carboxylic acids (e.g., acetic acid, propionic acid, trifluoroacetic acid, and trichloroacetic acid),
carboxylic acid esters (e.g., methyl acetate, ethyl acetate, propyl acetate, isopropyl acetate, butyl acetate and isomers thereof and pentyl acetate and isomers thereof (in the present invention, the “isomer of butyl acetate” is an equivalent of “butyl acetate” and the “isomer of pentyl acetate” is an equivalent of “pentyl acetate”)), ethers (e.g., tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME) and diglyme), ketones (e.g., acetone, methyl ethyl ketone (MEK), methyl isopropyl ketone (MIPK) and methyl isobutyl ketone (MIBK)), amides (e.g., N,N-dimethylformamide (DMF), N,N-dimethylacetamide (DMAC) and N-methylpyrrolidone (NMP)), ureas (e.g., N,N′-dimethylimidazolidinone (DMI) and tetramethylurea), sulfones (e.g., sulfolane), water, and any combination thereof in any ratio.

“2-Propanol” is referred to also as “isopropyl alcohol” or “isopropanol”.

Preferred examples of the solvent in the reaction in the step ii include combinations, in any ratio, of one or more (preferably one or two, and more preferably one) organic solvents selected from alcohols, nitriles, carboxylic acids, and amides with a water solvent.

From the same viewpoint as described above, preferred specific examples of the solvent in the reaction in the step ii include combinations, in any ratio, of one or more (preferably one or two, more preferably one) organic solvents selected from methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, propionitrile, butyronitrile, isobutyronitrile, succinonitrile, benzonitrile, acetic acid, propionic acid, trifluoroacetic acid, N,N-dimethylformamide (DMF), and N,N-dimethylacetamide (DMAC) with a water solvent.

From the same viewpoint as described above, more preferred specific examples of the solvent in the reaction in the step ii include combinations, in any ratio, of one or more (preferably one or two, more preferably one) organic solvents selected from methanol, ethanol, propanol, 2-propanol, butanol, acetonitrile, propionitrile, butyronitrile, isobutyronitrile, succinonitrile, benzonitrile, acetic acid, propionic acid, trifluoroacetic acid, and N,N-dimethylformamide (DMF) with a water solvent.

From the same viewpoint as described above, further preferred specific examples of the solvent in the reaction in the step ii include combinations, in any ratio, of one or more (preferably one or two, more preferably one) organic solvents selected from methanol, ethanol, propanol, acetonitrile, propionitrile, butyronitrile, isobutyronitrile, acetic acid, trifluoroacetic acid, and N,N-dimethylformamide (DMF) with a water solvent.

From the same viewpoint as described above, particularly preferred specific examples of the solvent in the reaction in the step ii include combinations, in any ratio, of one or more (preferably one or two, more preferably one) organic solvents selected from methanol, acetonitrile, acetic acid, and N,N-dimethylformamide (DMF) with a water solvent.

In either case, the solvent may be in a single layer or may be separated into two layers as long as the reaction proceeds. On the other hand, regarding the reaction system of the present invention, it has been estimated that acetonitrile is not preferred from the viewpoint of affinity between an organic solvent and a water solvent in the presence of a raw material and/or an intermediate (it has been suggested that the reaction may not sufficiently proceed). Contrary to the expectation, however, favorable results have been obtained.

In the step ii, when sulfuric acid is used in the reaction as described in Examples 2-1 to 2-18, examples of the organic solvent include, but are not limited to, the following:
aromatic hydrocarbon derivatives (e.g., benzene optionally substituted with one to three (preferably one or two, and more preferably one) selected from (C1-C4)alkyl groups and a chlorine atom, specifically, benzene, toluene, xylene, chlorobenzene, and dichlorobenzene),
halogenated aliphatic hydrocarbons (e.g., (C1-C4)alkane optionally substituted with 1 to 10 chlorine atoms, specifically, dichloromethane, and 1,2-dichloroethane (EDC)),
nitriles (e.g., (C2-C5)alkane nitriles, specifically acetonitrile),
carboxylic acid esters (e.g., (C1-C4)alkyl (C1-C6)carboxylate, specifically, for example, methyl acetate, ethyl acetate, propyl acetate, isopropyl acetate, butyl acetate and isomers thereof, pentyl acetate and isomers thereof, and hexyl acetate and isomers thereof; herein, for example, an “isomer of butyl acetate” being an equivalent of “butyl acetate”),
amides (e.g., N,N-di((C1-C4)alkyl) (C1-C4)alkaneamide and 1-(C1-C4)alkyl-2-pyrrolidone, specifically for example, N,N-dimethylformamide (DMF), N,N-dimethylacetamide (DMAC), N,N-diethylacetamide, and N-methylpyrrolidone (NMP)),
ureas (e.g., N,N′-dimethylimidazolidinone (DMI) and tetramethylurea), and
sulfones (e.g., sulfolane).

In one embodiment, the examples include preferably the following: aromatic hydrocarbon derivatives, halogenated aliphatic hydrocarbons, nitriles, carboxylic acid esters, and amides, and more preferably aromatic hydrocarbon derivatives, nitriles, carboxylic acid esters, and amides.

In another embodiment, the examples include preferably the following: benzene optionally substituted with one to three (preferably one or two, and more preferably one) selected from (C1-C4)alkyl groups and a chlorine atom, (C1-C4)alkane optionally substituted with 1 to 10 chlorine atoms, (C2-C5)alkane nitrile, (C1-C4)alkyl (C1-C6)carboxylate, N,N-di((C1-C4)alkyl) (C1-C4)alkaneamide, and 1-(C1-C4)alkyl-2-pyrrolidone, and more preferably benzene optionally substituted with one to three (preferably one or two, and more preferably one) selected from (C1-C4)alkyl groups and a chlorine atom, (C2-C5)alkane nitrile, (C1-C4)alkyl (C1-C6)carboxylate, N,N-di((C1-C4)alkyl) (C1-C4)alkaneamide, and 1-(C1-C4)alkyl-2-pyrrolidone.

In still another embodiment, the examples include preferably the following: benzene, toluene, xylene, chlorobenzene, dichlorobenzene, dichloromethane, 1,2-dichloroethane, acetonitrile, methyl acetate, ethyl acetate, propyl acetate, isopropyl acetate, butyl acetate and isomers thereof, pentyl acetate and isomers thereof, hexyl acetate and isomers thereof, N,N-dimethylformamide (DMF), N,N-dimethylacetamide (DMAC), N,N-diethylacetamide, and N-methylpyrrolidone (NMP), and more preferably toluene, xylene, chlorobenzene, dichlorobenzene, dichloromethane, 1,2-dichloroethane, acetonitrile, methyl acetate, ethyl acetate, propyl acetate, isopropyl acetate, butyl acetate and isomers thereof, pentyl acetate and isomers thereof, hexyl acetate and isomers thereof, N,N-dimethylformamide (DMF), N,N-dimethylacetamide (DMAC), N,N-diethylacetamide, and N-methylpyrrolidone (NMP), and further preferably toluene, xylene, chlorobenzene, dichlorobenzene, acetonitrile, methyl acetate, ethyl acetate, propyl acetate, isopropyl acetate, butyl acetate and isomers thereof, pentyl acetate and isomers thereof, hexyl acetate and isomers thereof, N,N-dimethylformamide (DMF), N,N-dimethylacetamide (DMAC), N,N-diethylacetamide, and N-methylpyrrolidone (NMP)), and still further preferably toluene, xylene, acetonitrile, ethyl acetate, propyl acetate, isopropyl acetate, butyl acetate and isomers thereof, pentyl acetate and isomers thereof, hexyl acetate and isomers thereof, N,N-dimethylformamide (DMF), N,N-dimethylacetamide (DMAC), N,N-diethylacetamide, and N-methylpyrrolidone (NMP).

In the step ii, in the reaction using sulfuric acid as described in Examples 2-1 to 2-18, (C1-C6)alcohols, particularly (C1-C4) alcohols are not preferred. This reaction is preferably performed in the absence of (C1-C6)alcohols, particularly (C1-C4)alcohols.

The (C1-C6)alcohol means (C1-C6)alkyl-OH (wherein the (C1-C6)alkyl moiety has the same meaning as defined above). Examples of the (C1-C4)alcohol include, but are not limited to, methanol, ethanol, propanol (i.e., 1-propanol), 2-propanol, butanol (i.e., 1-butanol), sec-butanol, isobutanol, tert-butanol, pentanol (i.e., 1-pentanol), sec-amyl alcohol, 3-pentanol, 2-methyl-1-butanol, isoamyl alcohol, tert-amyl alcohol, hexanol (i.e., 1-hexanol) and cyclohexanol.

The (C1-C4)alcohol means (C1-C4)alkyl-OH (wherein the (C1-C4)alkyl moiety has the same meaning as defined above). Examples of the (C1-C4)alcohol include, but are not limited to, methanol, ethanol, propanol (i.e., 1-propanol), 2-propanol, butanol, sec-butanol, isobutanol, and tert-butanol.

In still another embodiment, in the reaction using sulfuric acid as described in Examples 2-1 to 2-18 performed in the step ii, examples of the organic solvent include organic solvents having an acceptor number of 1 to 25, preferably 2 to 25, more preferably 2 to 20, and further preferably 2 to 19 in one embodiment. In another embodiment, examples of the organic solvent include organic solvents having an acceptor number of 5 to 25, preferably 5 to 20, more preferably 7 to 20, and further preferably 8 to 19.

In still another embodiment, in the reaction using sulfuric acid as described in Examples 2-1 to 2-18 performed in the step ii, examples of the organic solvent include organic solvents having a relative permittivity of 1 to 70, preferably 1 to 40, more preferably 2 to 40, and further preferably 2 to 38.

In still another embodiment, in the reaction using sulfuric acid as described in Examples 2-1 to 2-18 performed in the step ii, examples of the organic solvent include organic solvents having a Rohrschneider's polarity parameter of 1 to 7, and preferably 2 to 7.

(Acceptor Number)

Herein, regarding the acceptor number, for example, the following document can be referred to: Christian Reichardt, “Solvents and Solvent Effects in Organic Chemistry”, 3rd, updated and enlarged edition, WILEY-VCH, 2003, p. 25-26. The definition of the acceptor number utilizing 31P-NMR chemical shift values is described in the above document, which is incorporated into the present invention by reference. Examples of the solvent having the specific value are described in the document, which are incorporated into the present invention by reference.

(Relative Permittivity)

Herein, regarding the relative permittivity (generally known also as “dielectric constant”), for example, the following documents can be referred to: “Handbook of Chemistry (Pure Chemistry)”, Maruzen Co., Ltd., 5th revised edition, 2004, p. I-770-777, edited by the Chemical Society of Japan; and A. Maryott and Edgar R. Smith, National Bureau of Standards Circular 514, Table of Dielectric Constants of Pure Liquids, United States Department of Commerce, National Bureau of Standards, Aug. 10, 1951. These documents are incorporated into the present invention by reference. Examples of the solvent having the specific value are described in these documents, which are incorporated into the present invention by reference.

(Rohrschneider's Polarity Parameter)

Regarding the Rohrschneider's polarity parameter, for example, the following website can be referred to: https://www.shodex.com/ja/dc/06/0117.html. This is incorporated into the present invention by reference. Examples of the solvent having the specific value are described in the document, which is incorporated into the present invention by reference.

The “solvent in the reaction” refers to all organic solvents and water solvent used in the reaction. The “solvent in the reaction” does not include organic solvents and a water solvent used in the working-up (e.g., isolation and purification) after the reaction. The “organic solvent” used in the reaction includes the organic solvent in the raw material solution and that in the reactant solution. The “water solvent” used in the reaction includes the water in the raw material solution and that in the reactant solution (e.g., water in an aqueous hydrogen peroxide solution).

The amounts of the organic solvent and the water solvent used in the reaction in the step ii are not particularly limited as long as the reaction system can be sufficiently stirred. The amounts of the organic solvent and the water solvent used, and the ratio therebetween are, for example, in the ranges of any combination of the lower limits and the upper limits of the ranges thereof described herein.

From the viewpoint of yield, suppression of by-products, economic efficiency, etc., however, in one embodiment, the amount of the organic solvent used in the reaction in the step ii is, for example, 0 (zero) to 3 L (liters), preferably 0 (zero) to 2 L, and more preferably 0.4 to 1.8 L based on 1 mol of the compound of the formula (7) (raw material). The amount is, however, not limited thereto. In another embodiment, the amount of the organic solvent used in the reaction in the step ii is, for example, 0.1 to 5 L, and preferably 0.1 to 3 L based on 1 mol of the compound of the formula (7) (raw material). The amount is, however, not limited thereto.

From the same viewpoint as described above, the amount of the water solvent used in the reaction in the step ii is, for example, preferably 0.01 to 2 L (liters), more preferably 0.05 to 1 L, more preferably 0.1 to 0.5 L, and further preferably 0.1 to 0.3 L in one embodiment. The amount is, however, not limited thereto.

When a combination of two or more organic solvents is used, the ratio of the two or more organic solvents may be any ratio as long as the reaction proceeds. When a combination of an organic solvent and a water solvent is used, the ratio of the organic solvent to the water solvent may be any ratio as long as the reaction proceeds. In each process of the oxidation reaction of the present invention, however, preferred organic solvents and preferred amounts thereof, preferred amounts of the water solvent, and a preferred ratio therebetween have been found. These are as described herein.

(Reaction Temperature in Step 11)

The reaction temperature in the step ii is not particularly limited. However, from the viewpoint of yield, suppression of by-products, economic efficiency, etc., the reaction temperature is, for example, a range of any combination of lower limits and upper limits of the following ranges. In one embodiment, the reaction temperature in the step ii is, for example, 0 (zero)° C. to 100° C., preferably 30° C. to 100° C., more preferably 30° C. to 80° C., further preferably 40° C. to 80° C., and further preferably 40° C. to 60° C. In another embodiment, the reaction temperature in the step ii is, for example, 40° C. to 100° C., preferably 45° C. to 100° C., and more preferably 45° C. to 80° C. In still another embodiment, the reaction temperature in the step ii is, for example, 0 (zero)° C. to 80° C., preferably 5° C. to 60° C., more preferably 5° C. to 50° C., further preferably 5° C. to 40° C., and further preferably 10° C. to 40° C.

In still another embodiment, in the reaction using sulfuric acid as described in Examples 2-1 to 2-18 performed in the step ii, the reaction temperature is 30° C. to 100° C., preferably 35° C. to 90° C., and more preferably 40° C. to 80° C., and in still another embodiment, in the reaction using sulfuric acid, the reaction temperature is 35° C. to 100° C., 35° C. to 110° C., 35° C. to 120° C., 35° C. to 150° C., 40° C. to 150° C., 60° C. to 150° C., or 70° C. to 150° C.

(Reaction Time in Step ii)

The reaction time in the step ii is not particularly limited. However, from the viewpoint of yield, suppression of by-products, economic efficiency, etc., in one embodiment, the reaction time in the step ii is, for example, 5 minutes to 48 hours, preferably 10 minutes to 24 hours, and more preferably 10 minutes to 12 hours. In another embodiment, the reaction time in the step ii is, for example, 1 hour to 48 hours, preferably 1 hour to 24 hours, and more preferably 30 minutes to 12 hours. The reaction time can be, however, appropriately adjusted by a person skilled in the art.

(Adding Method in Step ii)

The order of adding the raw material, the oxidizing agent, the acidic compound, the base, the solvent, etc. is not particularly limited. As long as the reaction proceeds, the addition order thereof may be any order.

(Adding Method in Step ii: Process Using Base)

In a process where a base is used in the step 11, the order of adding the raw material, the base, and the oxidizing agent may be any order as long as the reaction proceeds. From the viewpoint of yield, etc., however, “batch addition” or “simultaneous addition of the base and the oxidizing agent” is preferred. From the viewpoint of yield, suppression of by-products, economic efficiency, safety, etc., the “simultaneous addition of the base and the oxidizing agent” is more preferred. In employing the “simultaneous addition of the base and the oxidizing agent”, the compound of the formula (7) of the raw material is added before starting the “simultaneous addition of the base and the oxidizing agent”. In this case, however, a part of the compound of the formula (7) of the raw material may be added during the “simultaneous addition of the base and the oxidizing agent”.

(Addition Rate of Base in Step ii)

In the “simultaneous addition of the base and the oxidizing agent”, from the viewpoint of yield, suppression of by-products, economic efficiency, safety, etc., the addition rate of the base in the step ii is, for example, within a range of any combination of lower limits and upper limits of the following ranges. The addition rate of the base in the step ii is, for example, 0.01 mol/hr. to 1 mol/hr., preferably 0.01 mol/hr. to 0.7 mol/hr., more preferably 0.01 mol/hr. to 0.6 mol/hr., further preferably 0.01 mol/hr. to 0.5 mol/hr., further preferably 0.02 mol/hr. to 0.5 mol/hr., and still further preferably 0.03 mol/hr. to 0.5 mol/hr. based on 1 mol of the compound of the formula (7).

(Addition Rate of Oxidizing Agent in Step ii)

In the “simultaneous addition of the base and the oxidizing agent”, from the viewpoint of yield, suppression of by-products, economic efficiency, safety, etc., the addition rate of the oxidizing agent in the step ii is, for example, within a range of any combination of lower limits and upper limits of the following ranges. In one embodiment, the addition rate of the oxidizing agent in the step ii is, for example, 0.06 mol/hr. to 2 mol/hr., preferably 0.1 mol/hr. to 1.5 mol/hr., and more preferably 0.13 mol/hr. to 1 mol/hr. based on 1 mol of the compound of the formula (7). In another embodiment, the addition rate of the oxidizing agent in the step ii is, for example, 0.05 mol/hr. to 6 mol/hr., preferably 0.05 mol/hr. to 5 mol/hr., more preferably 0.1 mol/hr. to 5 mol/hr., and further preferably 0.2 mol/hr. to 5 mol/hr. based on 1 mol of the compound of the formula (7).

(Relationship in Addition Rate Between Base and Oxidizing Agent in Step ii)

In employing the “simultaneous addition of the base and the oxidizing agent”, from the viewpoint of yield, suppression of by-products, economic efficiency, safety, etc., it is preferable that the addition rate of the base in the step ii is the same as the addition rate of the oxidizing agent in the step ii, or that the addition rate of the oxidizing agent in the step ii is higher than the addition rate of the base in the step ii, and it is more preferable that the addition rate of the oxidizing agent in the step ii is higher than the addition rate of the base in the step ii. For example, the addition rate of the oxidizing agent in the step ii is 1 time to 30 times (preferably over 1 time and 30 times or less), 1 time to 20 times (preferably over 1 time and 20 times or less), or 1 time to 10 times (preferably over 1 time and 10 times or less) the addition rate of the base in the step ii.

(Addition Time and Aging Time of Base and Oxidizing Agent in Step ii)

In the “simultaneous addition of the base and the oxidizing agent”, from the viewpoint of yield, suppression of by-products, economic efficiency, safety, etc., the addition time of the base and the oxidizing agent in the step ii is preferably 0.5 hours or more, more preferably 0.75 hours or more, and further preferably 1 hour or more. The addition time of the base in the step ii is, for example, 1 hour to 48 hours, preferably 1 hour to 24 hours, and more preferably 1 hour to 12 hours. From the same viewpoint as described above, the addition time of the oxidizing agent in the step ii is, for example, 1 hour to 48 hours, preferably 1 hour to 24 hours, and more preferably 1 hour to 12 hours. From the same viewpoint as described above, the aging time after the addition in the step ii is, for example, 0.1 hours to 24 hours, preferably 0.1 hours to 12 hours, more preferably 0.2 hours to 9 hours, and further preferably 0.5 hours to 6 hours.

(Addition Time, Aging Time, and Reaction Time)

Herein, “aging time” refers to stirring time after completing the addition of the raw material and/or the reactant (e.g., hydrogen peroxide, the acidic compound, and the base). When the “batch addition” is employed as the method for adding the raw material, the reactants and the like, the “reaction time” corresponds to the “aging time”.

When the raw material and/or the reactants and the like are added over a prescribed period of time, the “addition time” refers to time from the start of the addition of the raw material and/or the reactants such as hydrogen peroxide and the base to the completion of the addition of the whole amounts thereof. Also in this case, the “aging time” corresponds to stirring time after completing the addition of the raw material and/or the reactants. In this case, it is estimated that the reaction starts after starting the addition, and the “reaction time” is a sum total of the “addition time” and the “aging time”.

(Adding Method in Step ii: Process Using Both Acidic Compound and Base)

Alternatively, the oxidation reaction in the step ii may be performed using an acidic compound and a base.

In one embodiment, a compound of the formula (8) can be produced by reacting the compound of the formula (7) with an oxidizing agent under acidic conditions, and then reacting the resultant with an oxidizing agent under neutral to alkaline conditions.

In another embodiment, the compound of the formula (8) can be produced by reacting the compound of the formula (7) with an oxidizing agent in the presence of an acidic compound, and then reacting the resultant with an oxidizing agent under neutral to alkaline conditions.

In still another embodiment, the compound of the formula (8) can be produced by reacting the compound of the formula (7) with an oxidizing agent in the presence of an acidic compound, and then reacting the resultant with an oxidizing agent using a base.

Herein, the term “in the presence of an acidic compound” can be optionally replaced by the term “under acidic conditions”. The term “under neutral to alkaline conditions” can be optionally replaced by the term “using a base”.

Under the acidic conditions using an acidic compound, in one embodiment, for example, the pH value is in the range of 6.0 or less, preferably larger than 0 and 5.5 or less, more preferably larger than 0 and 5.0 or less, further preferably larger than 0 and 4.0 or less, and still further preferably larger than 0 and 3.0 or less. In another embodiment, for example, the pH value is in the range of 6.0 or less, preferably larger than −1 and 5.5 or less, more preferably larger than −1 and 5.0 or less, further preferably larger than −1 and 4.0 or less, and still further preferably larger than −1 and 3.0 or less.

Under the neutral to alkaline conditions, in one embodiment, for example, the pH value is in the range of 6.0 or more, preferably 6.5 to 14.0, more preferably 7.0 to 12.0, and further preferably 8.0 to 10.0. In another embodiment, for example, the pH value is 7.0 or more, preferably 7.5 to 14.0, more preferably 8.0 to 12.0, and further preferably 8.5 to 10.0.

(Embodiments of Reaction)

The present reaction can be performed by a batch method using a reaction kettle, or alternatively, can be performed through a flow reaction using a continuous reactor. The continuous reactor refers to a reactor used for causing raw material supply and the reaction to continuously and simultaneously proceed. An example of the continuous reactor includes a flow reactor. A flow reactor is a reactor capable of performing reaction continuously with a raw material continuously supplied thereto. A flow reactor is roughly divided into a tubular flow reactor (including a tube flow reactor), and a tank flow reactor, both of which can perform a reaction by a continuous method. The flow reactor of the present invention may be provided with temperature control means for controlling the temperature of the flow reactor, and may be provided with, for example, a temperature control unit for heating and cooling. The temperature control unit may be any suitable unit, and examples of the temperature control unit include a bath and a jacket. The bath and the jacket may be in any suitable form. Besides, the material of the flow reactor is not particularly limited as long as it is unaltered by a raw material and a solvent, and examples include metals (e.g., titanium, nickel, stainless steel, and Hastelloy C), resins (e.g., fluororesin), glass, and porcelain (e.g., ceramics).

It is not excluded that the continuous reaction of the present invention is performed with a tank flow reactor. A preferred example of the flow reactor includes, however, a tubular flow reactor. The tubular flow reactor of the present invention may be any reactor capable of causing a liquid or a vapor-liquid mixture to continuously flow therethrough, and the cross-sectional shape of the tube may be any one of circular, rectangular, polygonal, and elliptical tubular shapes, or a shape of a combination of these shapes. Besides, the material of the tube is not particularly limited as long as it is unaltered by a raw material and a solvent, and examples include metals (e.g., titanium, nickel, stainless steel, and Hastelloy C), resins (e.g., fluororesin), glass, and porcelain (e.g., ceramics), and preferably, fluororesin (e.g., Teflon (registered trademark)) is preferred. Also the tubular flow reactor of the present invention may be provided with temperature control means for controlling the temperature, and may be provided with, for example, a temperature control unit for heating and cooling. The temperature control unit may be any suitable unit, and examples of the temperature control unit include a bath and a jacket. The bath and the jacket may be in any suitable form. As such a flow reactor, for example, spiral, shell-and-tube, and plate heat exchanger reactors can be used.

A layout method for the tube in the tubular flow reactor of the present invention is not particularly limited, and for example, may be linear layout, curved layout, or coil layout. A preferred example of the layout method includes a tubular reactor having a tube in a coil layout. Besides, the number of tube may be one, or a plurality of two or more tubes may be regularly or irregularly bundled at appropriate intervals. Herein, a tubular flow reactor having one tube is used in the description for convenience, and if production efficiency is desired to be increased, a tubular flow reactor in which a plurality of two or more tubes are regularly or irregularly bundled at appropriate intervals may be used in accordance with the description provided herein.

Besides, the tubular flow reactor of the present invention may include a mixer as desired. The mixer is not particularly limited as long as it has a function capable of continuously mixing two or more fluids, such as a gas and a liquid, or a liquid and a liquid, and examples include a Y-shaped mixer, a T-shaped mixer, and a pipeline mixer (line mixer including a static mixer). A line mixer including a static mixer or the like may be a tubular flow reactor.

(Reaction by Flow Method)

When the flow method is employed, a mixture of prescribed amounts of the compound (7), an acidic compound (or a base), hydrogen peroxide and a solvent (with another component added if necessary) is caused to flow through a tubular reactor for causing a reaction. In this case, it is preferable that the tubular reactor to be used has a heater, and that the mixture is caused to flow through the reaction tube heated to a prescribed temperature. The reaction temperature is not particularly limited. From the viewpoint of yield, suppression of by-products, economic efficiency, etc., the reaction temperature is in the range of, for example, 0° C. (zero) to 120° C., and preferably 30° C. to 100° C.

The equivalent diameter of the tube in the tubular reactor of the present invention is not particularly limited as long as a liquid or vapor-liquid mixture can continuously flow therethrough, and also from the viewpoint of production efficiency, it is preferably 0.5 mm or more. A preferred example of the equivalent diameter includes 0.5 mm to 50 mm, and preferably about 0.5 mm to 30 mm.

The “equivalent diameter (De)” of the present invention is a value defined in accordance with the following equation:

De=4·Af/Wp
wherein Af indicates a tube cross-sectional area, and Wp indicates a wetted perimeter.

For example, the equivalent diameter of a circular tube having a radius r is:

$$\begin{matrix}De = 4 \cdot π {r}^{2} / 2 π r \\ = 2 r\end{matrix}$$

The length of the tube of the tubular flow reactor of the present invention is not particularly limited as long as a raw material compound can be heated and sufficiently reacted therein. The length is, for example, 1 m or more, and preferably in the range of 5 m to 80 m. In order to efficiently perform the process of the present invention, since it is necessary to cause a reaction at a prescribed temperature, and/or for ensuring a sufficient reaction time, the length is, but is not limited to, preferably 5 m or more in general.

The flow rate in the flow reactor, preferably in the tubular flow reactor of the present invention depends on the equivalent diameter of the tube, and is usually 0.01 mL/min or more, and preferably 0.05 mL/min or more.

The pressure within the tubular flow reactor is, but is not limited to, for example, 0.1 MPa to 10 MPa, and preferably 0.3 MPa to 5 MPa.

(Working-Up in Step ii; Isolation and Purification)

The compounds of the formula (8), especially pyroxasulfone (8-a), which is the target product in the step ii, can be isolated and purified from the reaction mixture by methods known to a person skilled in the art (e.g., extraction, washing, crystallization including recrystallization, crystal washing and/or other procedures) and improved methods thereof, and any combination thereof.

In the step ii, as shown in Examples, it is preferable to decompose an unreacted peroxide such as hydrogen peroxide by treating the reaction mixture with a reducing agent (e.g., an aqueous sodium sulfite solution) after the reaction.

In the working-up step (isolation and/or purification), the following procedures may be performed, but are not limited thereto: in the working-up, an extraction procedure and/or a washing procedure including separation of an organic layer and an aqueous layer may be performed. When the mixture is separated into an organic layer and an aqueous layer, the mixture may be separated while being hot. For example, when separating the organic layer from the aqueous layer, a hot mixture may be used, or the mixture may be heated. Impurities may be removed by a filtration procedure including hot filtration.

In the working-up, crystallization of the target product including recrystallization and washing of crystals may be performed. The crystallization of the target product including recrystallization may be performed by a conventional method known to a person skilled in the art. For example, an antisolvent may be added to a solution of the target product in a good solvent. As another example, a saturated solution of the target product may be cooled.

For still another example, from the solution of the target product in an organic solvent (including the reaction mixture), the solvent may be removed. In this case, examples of the organic solvent that can be used include the examples, the preferred examples, the more preferred examples, and the further preferred examples of the water-miscible organic solvent described later. The organic solvent may be removed after adding water in advance into the system. In this case, the organic solvent may be removed by azeotropy with the water. The organic solvent may be removed under heating, under reduced pressure and under normal pressure. As still another example, water may be added to a solution of the target product in a water-miscible organic solvent. Examples of the water-miscible organic solvent include, but are not limited to, alcohols (e.g., methanol, ethanol, 2-propanol, butanol and t-butanol), nitriles (e.g., acetonitrile), ethers (e.g., tetrahydrofuran (THF) and 1,4-dioxane), ketones (e.g., acetone), amides (e.g., N,N-dimethylformamide (DMF), N,N-dimethylacetamide (DMAC) and N-methylpyrrolidone (NMP)), sulfoxides (e.g., dimethyl sulfoxide (DMSO)), and combinations thereof, preferably methanol, ethanol, 2-propanol, butanol, acetonitrile, acetone, and combinations thereof, and more preferably ethanol, 2-propanol, butanol, acetonitrile, and combinations thereof. The “water-miscible organic solvent” has the same meaning as “water-soluble organic solvent”. “2-Propanol” is also referred to as “isopropyl alcohol” or “isopropanol”.

In any of the above cases, a seed crystal may be used.

In the crystal washing procedure, the crystals collected by filtration may be washed with a solvent. A suspension (slurry) of crystals may be stirred and then filtered. In any case, examples of the solvent that can be used include the examples, the preferred examples, the more preferred examples, the further preferred examples of the water-miscible organic solvent described above and water.

In any of the above cases (crystallization procedures including recrystallization, crystal washing procedure, etc.), the amount of the solvent such as the water-miscible organic solvent and the amount of water may be at any ratio as long as the purpose is achieved. When a combination of a water-miscible organic solvent and water is employed, the ratio thereof may be any ratio as long as the purpose is achieved. When a combination of two or more solvents such as water-miscible organic solvents is employed, the ratio thereof may be any ratio as long as the purpose is achieved. Their amounts and ratios can be appropriately adjusted by a person skilled in the art depending on the purpose and situation.

In any of the above procedures (extraction procedure, washing procedure, crystallization procedures including recrystallization, crystal washing procedure, etc.), the temperature can be appropriately adjusted by a person skilled in the art. However, from the viewpoint of yield, purity, economic efficiency, etc., for example, the temperature is 0° C. (zero ° C.) to 100° C., preferably 5° C. to 90° C., and more preferably 10° C. to 80° C. Heating and cooling may be performed in these temperature ranges.

In any of the above procedures (extraction procedure, washing procedure, crystallization procedures including recrystallization, crystal washing procedure, etc.), the amount of the organic solvent (including the water-miscible organic solvent) and/or water can be appropriately adjusted by a person skilled in the art by addition and removal thereof. Furthermore, recovery and recycling of the solvent may be optionally performed. For example, the recovery and recycle of the solvent used in the reaction may be performed, and the recovery and recycle of the solvent used in the working-up (isolation and/or purification) may be performed.

Working-up (isolation and/or purification) can be performed by appropriately combining all or some of the procedures described above. Optionally, the above procedures may be repeated according to the purpose such as isolation and/or purification. In addition, a person skilled in the art can appropriately select a combination of any of the above procedures and their order.

Hereinafter, the present invention will be described in more detail by Examples, but the present invention is not limited in any way by these Examples.

In the present description, the following instruments and conditions were used for the determination of physical properties and yields in Examples, Comparative Examples and Reference Examples. In addition, the products obtained in the present invention are known compounds, and were identified in the usual manner known to a person skilled in the art.

(Measurement of pH)
Instrument: as a glass electrode type hydrogen ion concentration meter, HM-20P manufactured by DKK-TOA CORPORATION or any equivalent thereto

(HPLC Analysis: High Performance Liquid Chromatography Analysis)

(HPLC Analysis Conditions)
Instrument: LC 2010 Series manufactured by Shimadzu Corporation or any equivalent thereto
Column: YMC-Pack, ODS-A, A-312 (150 mm×6.0 mm ID, S-5 μm, 120 A)
Eluent:

TABLE 1

| | | 0.1% Aqueous phosphoric |
|---|---|---|
| Time (min) | Acetonitrile (%) | acid solution (%) |
| 0 | 45 | 55 |
| 10 | 45 | 55 |
| 15 | 80 | 20 |
| 20 | 80 | 20 |

Flow rate: 1.0 ml/min
Detection: UV 230 nm
Column temperature: 40° C.
Injection volume: 5 μL

The following documents can be referred to for the HPLC analysis method, as desired.
Literature (a): “Shin Jikkenkagaku Koza 9 (A New Course in Experimental Chemistry Course 9) Bunsekikagaku II (Analytical Chemistry II)”, pages 86 to 112 (1977), edited by the Chemical Society of Japan, published by Shingo Iizumi, Maruzen Co., Ltd.
Literature (b): “Jikkenkagaku Koza 20-1 (A Course in Experimental Chemistry 20-1), Bunseki Kagaku (Analytical Chemistry)”, 5th edition, pages 130 to 151 (2007), edited by the Chemical Society of Japan, published by Seishiro Murata, Maruzen Co., Ltd.

(Yield and Purity)

Unless otherwise specified, the yield in the present invention can be calculated from the number of moles of the obtained target compound with respect to the number of moles of the raw material compound (starting compound).

That is, the term “yield” means “molar yield”.

Thus, the yield is represented by the following equation:

Yield (%)=(the number of moles of the target compound obtained)/(the number of moles of the starting compound)×100.

However, for example, in the evaluation of the reaction yield of the target product, the yield of impurities, the purity of the product, etc., HPLC area percentage analysis or GC area percentage analysis may be employed.

Herein, room temperature and ordinary temperature are from 10° C. to 30° C. Herein, “RT”, “rt”, “r.t” and “r.t.” means room temperature.

Herein, the term “overnight” means from 8 hours to 16 hours.

Herein, the procedure of “age/aged/aging” includes stirring a mixture by the usual manner known to a person skilled in the art.

In Examples described herein, “sulfuric acid” means concentrated sulfuric acid unless otherwise specified. An example of the concentrated sulfuric acid includes, but is not limited to, 98% sulfuric acid.

### EXAMPLES

### Example 1

Production of 3-[(5-difluoromethoxy-1-methyl-3-trifluoromethylpyrazol-4-yl)methylthio]-4,5-dihydro-5,5-dimethylisoxazole (Compound 7-a)

### Example 1-1

(Step Pre-i-a)

### Production of 4-chloromethyl-5-difluoromethoxy-1-methyl-3-trifluoromethylpyrazole (Compound 1-a)

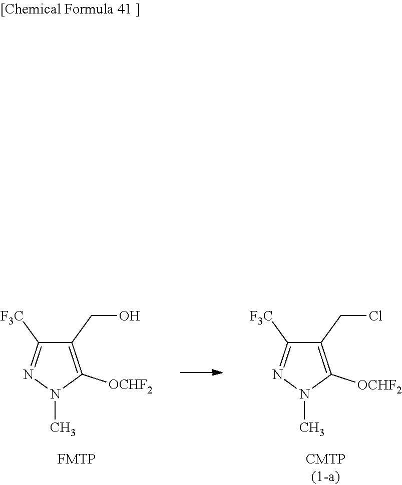 [SMILES: Cn1nc(C(F)(F)F)c(CO)c1OC(F)F ; Cn1nc(C(F)(F)F)c(CCl)c1OC(F)F]

To 5-difluoromethoxy-4-hydroxymethyl-1-methyl-3-trifluoromethylpyrazole (46.7 g, purity: 68.6%, containing acetonitrile, 0.13 mol, 100 mol %) was added thionyl chloride (17.0 g, 0.14 mol, 110 mol %) dropwise at an internal temperature of 20° C. to 30° C. over 1 hour. After the dropwise addition, the mixture was aged at an internal temperature of 20° C. to 30° C. for 1 hour. After the completion of the reaction, nitrogen was blown into the reaction mixture for 30 minutes to remove the excess thionyl chloride, and ethyl acetate (78 mL, 0.6 L/mol) was added thereto. The obtained solution of the title compound (1-a) in ethyl acetate weighed 134 g.

### Example 1-2

(Step i-a)

### Production of 3-[(5-difluoromethoxy-1-methyl-3-tri fluoromethylpyrazol-4-yl)methylthio]-4,5-dihydro-5,5-dimethylisoxazole (Compound 7-a)

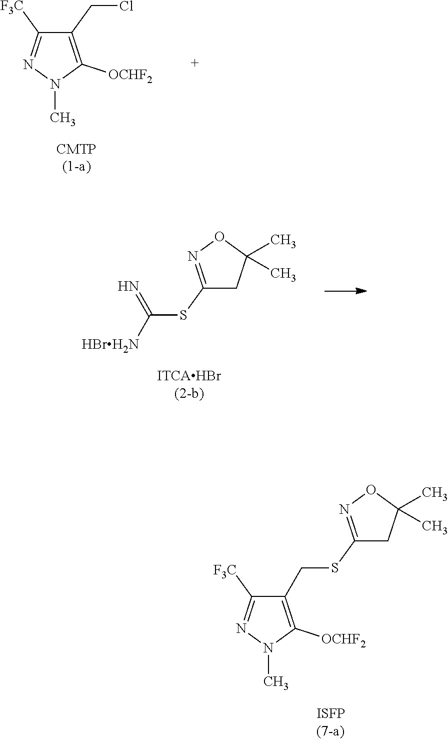

The solution (134 g, corresponding to 0.13 mol scale) of 4-chloromethyl-5-difluoromethoxy-1-methyl-3-trifluoromethyl-pyrazole (1-a) in ethyl acetate produced in the step pre-i-a was cooled to an internal temperature of 10° C. or lower with ice-cooling and stirring. To this was added an aqueous solution (134.6 g, purity: 27%, equivalent to 0.14 mol) of (5,5-dimethyl[4,5-dihydroisoxazolo-3-yl)]thiocarboxamidine hydrobromide (2-b), and then a 48% aqueous sodium hydroxide solution (54.2 g, 0.65 mol, 500 mol %) was added dropwise over 30 minutes such that the internal temperature did not exceed 10° C. After the dropwise addition, the mixture was aged at an internal temperature of 10° C. or lower for 30 minutes, then warmed to an internal temperature of 25° C. and aged for 4 hours. After the completion of the reaction, the reaction mixture was separated into an organic layer and an aqueous layer. The obtained organic layer was analyzed by the HPLC absolute calibration curve method. As a result, the yield of the target product (7-a) was 91.6% (127.8 g, through 2 steps).

### Example 1-3

(Step Pre-i-b)

### Production of 3-[(5-hydroxy-1-methyl-3-trifluoromethylpyrazol-4-yl)methylthio]-4,5-dihydro-5,5-dimethylisoxazole (Compound 4-a)

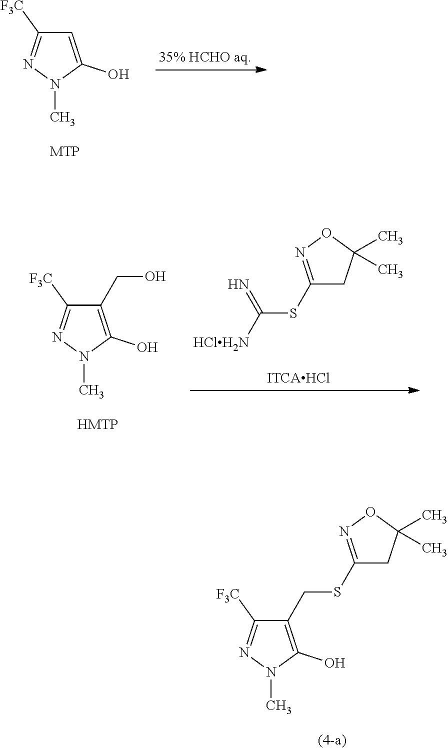

5-Hydroxy-1-methyl-3-trifluoromethylpyrazole (MTP) (1.7 g, 10.00 mmol, 100 mol %) and 1.6 g of sodium hydroxide (40.00 mmol, 400 mol %) were dissolved in 10 ml of water. While the resultant solution being stirred at room temperature, 1.7 g (20 mmol) of a 35% aqueous formaldehyde solution (35% formalin solution) was added dropwise thereto, followed by stirring at the same temperature for 1 hour. To the resultant, a solution of 2.1 g (10.00 mmol) of [5,5-dimethyl(4,5-dihydroisoxazol-3-yl)]thiocarboxamidine hydrochloride (ITCA/HCl, 2-a) in 10 ml of water was added dropwise at room temperature, followed by stirring for 2 hours. After the reaction, 5.0 g (50 mmol) of 35% hydrochloric acid was added dropwise thereto. The thus precipitated crystals were suction filtered, and washed with 5 mL of water twice. The resultant was dried with a hot air dryer to obtain 2.5 g of a compound (4-a) as pale yellow crystals. The yield was 80.1%.

### Example 1-4

(Step i-b)

### Production of 3-[(5-difluoromethoxy-1-methyl-3-trifluoromethylpyrazol-4-yl)methylthio]-4,5-dihydro-5,5-dimethylisoxazole (Compound 7-a)

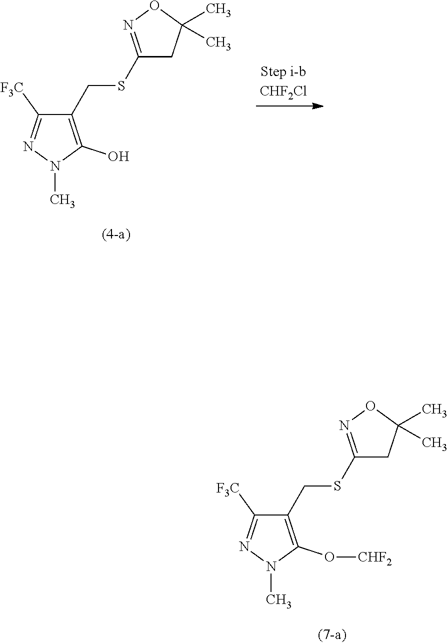 [SMILES: Cn1nc(C(F)(F)F)c(CSC2=NOC(C)(C)C2)c1O ; Cn1nc(C(F)(F)F)c(CSC2=NOC(C)(C)C2)c1OC(F)F]

To 100 ml of acetonitrile, 33.2 g (purity: 93.3%, 0.1 mol) of 3-[(5-hydroxy-1-methyl-3-trifluoromethylpyrazol-4-yl)methylthio]-4,5-dihydro-5,5-dimethylisoxazole synthesized in Example 1-4 and 12.0 g (0.3 mol) of 99% sodium hydroxide were added, followed by stirring at room temperature for 1 hour. The resultant suspension was cooled on ice, and with the temperature of 5 to 15° C. maintained, 17.3 g (0.2 mol) of chlorodifluoromethane was introduced thereinto over 4 hours to perform a reaction within the same temperature range for 5 hours. After completing the reaction, 100 mol of toluene, 50 ml of water, and 10 ml of 35% hydrochloric acid were added thereto to collect an organic layer. An aqueous layer was re-extracted with 50 ml of toluene, and the combined organic layer was washed successively with 50 ml of water and 20 ml of saturated saline. The thus obtained organic layer was dried over sodium sulfate, and the solvent was distilled off to obtain 38.0 g of a compound (7-a) with a purity of 85%. The yield was 90%.

### Example 1-5

(Step Pre-i-c)

### Production of (5-difluoromethoxy-1-methyl-3-trifluoromethyl-1H-pyrazol-4-ylmethyl)isothiourea hydrobromide (Compound 5-b)

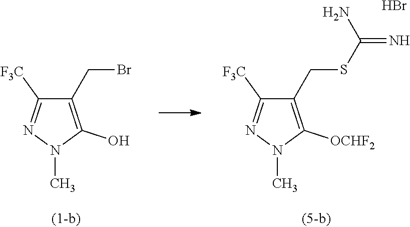 [SMILES: Br ; Cn1nc(C(F)(F)F)c(CBr)c1O ; Cn1nc(C(F)(F)F)c(CSC(=N)N)c1OC(F)F]

To 30 mL of a solution of 4-bromomethyl-5-difuoromethoxy-1-methyl-3-trifluoromethyl-pyrazole (1-b; purity: 75.0%, 46.3 mmol) in ethanol, 3.5 g (46.3 mmol) of thiourea was added, the resultant was stirred under heating to reflux for 1 hour, the solvent was distilled off under reduced pressure, and the resultant was washed with a mixed solvent of ethyl acetate and n-hexane to obtain 13.8 g of white crystals of the target product (5-b). The yield was 77.5%.

(Step i-c)

### Production of 3-(5-difluoromethoxy-1-methyl-3 trifluoromethylpyrazol-4-yl)methylthio)-4,5-dihydro-5,5-dimethylisoxazole (Compound 7-a)

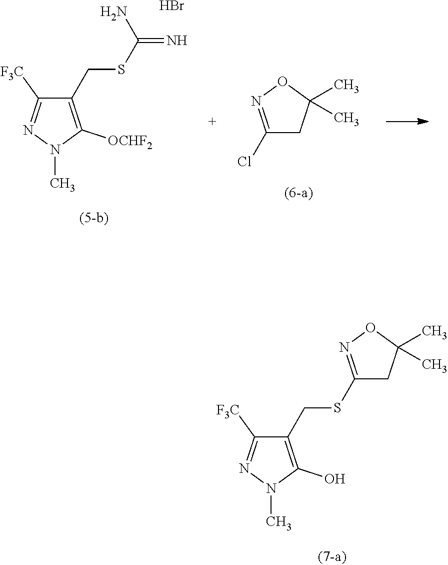

To 10 mL of a solution of (5-difluoromethoxy-1-methyl-3-trifluoromethyl-1H-pyrazol-4-ylmethyl) isothiourea hydrobromide (1.93 g, 5.00 mmol) in ethanol, 0.48 g (12.00 mmol) of sodium hydroxide and 10 ml of water were added, followed by stirring at room temperature for 30 minutes. To the resultant, 0.67 g (5.00 mmol) of 3-chloro-5,5-dimethyl-2-isoxazoline was added at room temperature, followed by stirring under reflux for 12 hours. After the completion of the reaction was confirmed, the solvent was distilled off under reduced pressure. The thus obtained residue was poured into water and extracted with ethyl acetate. The thus obtained organic layer was washed with water, and dried over anhydrous magnesium sulfate. The solvent was distilled off under reduced pressure, and the residue was purified by silica gel column chromatography to obtain 1.02 g of the target product (7-a). The yield was 56.7%.

### Reference Example 1

### Production of [5,5-dimethyl(4,5-dihydroisoxazolo-3-yl)]thiocarboxamidine hydrobromide

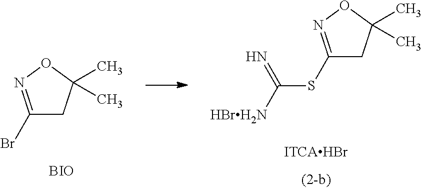

Thiourea (20 g, 0.26 mol, 105 mol %) was added to a solution of 3-bromo-5,5-dimethyl-4,5-dihydroisoxazole (BI) obtained by a process described in WO 2006/038657A in butyl acetate (251.5 g, purity: 18%, 0.25 mol), and the internal temperature was adjusted to 15° C. to 25° C. To this was added 35% hydrochloric acid (26 g, 0.25 mol, 100 mol %) dropwise at an internal temperature of 15° C. to 25° C. over 30 minutes. After the dropwise addition, the mixture was aged at an internal temperature of 15° C. to 25° C. for 6 hours. After the completion of the reaction, water (88 g, 0.35 L/mol) was added, the mixture was stirred for 15 minutes, and the reaction mixture was separated into an organic layer and an aqueous layer. Water (25 g, 0.1 L/mol) was added to the obtained organic layer, the mixture was stirred for 15 minutes, and the reaction mixture was separated into an organic layer and an aqueous layer. The obtained aqueous layers were combined to afford 208.6 g of an aqueous solution containing the target product corresponding to a yield of 90%. The obtained target product contained a hydrobromide derived from the raw material BIO and hydrochloride derived from hydrochloric acid.

### Example 2-1

### Production of 3-[(5-difluoromethoxy-1-methyl-3-trifluoromethylpyrazol-4-yl)methylsulfonyl]-4,5-dihydro-5,5-dimethylisoxazole

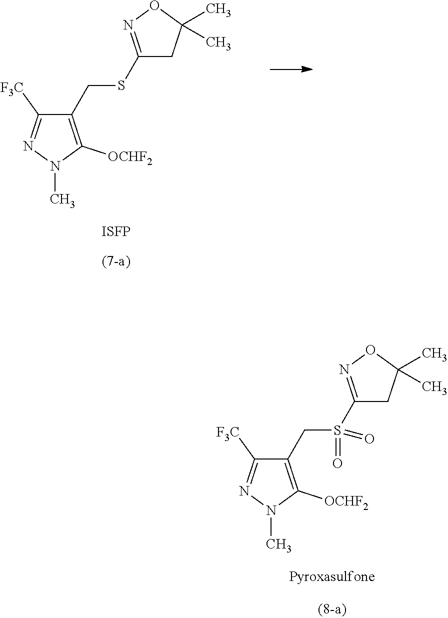

Under a nitrogen stream, the compound (7-a) (0.90 g, purity: 100%, 2.5 mmol, 100 mol %), 2.94 g (1.5 L/mol) of acetonitrile, sulfuric acid (0.77 g, 7.50 mmol, 300 mol %), a 35% aqueous hydrogen peroxide solution (0.81 g, 7.12 mmol, 285 mol %, containing 0.57 g (0.2 L/mol) of water) were added to a reaction flask, followed by stirring at 75° C. for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 86%.

### Examples 2-2 to 2-6 and Comparative Examples 1 to 3

The reaction and the analysis were performed in the same manner as in Example 2-1 except that the amount of the acetonitrile solvent, the amount of the sulfuric acid, the reaction temperature and the aging time were changed as shown in Table 2. The results are shown in Table 2. In addition, the results of Example 2-1 are also summarized in Table 2.

TABLE 2

| | Amount of | Amount of | | | HPLC area % (230 | | | |
|---|---|---|---|---|---|---|---|---|
| | acetonitrile | sulfuric | Reaction | Aging | nm) of component | | | Yield |
| | solvent | acid | temperature | time | in reaction mixture | | | (%) |
| Example No. | (L/mol) | (mol %) | (° C.) | (h) | (7-a) | (9-a) | (8-a) | (8-a) |
| 2-1 | 1.5 | 300 | 75 | 6 | 0 | 0 | 94.2 | 86 |
| 2-2 | 1.5 | 300 | 40 | 20 | 0 | 1.8 | 93.8 | 87 |
| Comparative Example 1 | 1.5 | 300 | r.t. | 6 | 59.4 | 37.3 | 0 | — |
| Comparative Example 2 | 0.5 | 10 | 75 | 24 | 0 | 55.3 | 41.0 | — |
| Comparative Example 3 | 0.5 | 20 | 75 | 28 | 0 | 8.8 | 88.0 | 82 |
| 2-3 | 0.5 | 30 | 75 | 28 | 0 | 0.7 | 95.7 | 89 |
| 2-4 | 0.5 | 50 | 75 | 18 | 0 | 0.9 | 96.3 | 90 |
| 2-5 | 1.5 | 50 | 75 | 24 | 0 | 2.0 | 93.6 | 87 |
| 2-6 | 1.5 | 200 | 75 | 2 | 0 | 0.3 | 95.3 | 84 |

### Example 2-7

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 2.94 g (1.5 L/mol) of toluene, sulfuric acid (0.77 g, 7.50 mmol, 300 mol %), and a 30% aqueous hydrogen peroxide solution (0.81 g, 7.12 mmol, 285 mol %, containing 0.57 g (0.2 L/mol) of water) were added to a reaction flask, followed by stirring at 75° C. for aging for 15 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 91%.

### Examples 2-8 to 2-18 and Comparative Examples 4 to 7

The reaction and the analysis were performed in the same manner as in Example 2-7 except that the organic solvent and the amount thereof, the amount of the sulfuric acid, the reaction temperature and the aging time were changed as shown in Table 3. The results are shown in Table 3. In addition, the results of Example 2-7 are also summarized in Table 3.

TABLE 3

| | | Amount of | Amount of | | | HPLC area % (230 | | | |
|---|---|---|---|---|---|---|---|---|---|
| | | organic | sulfuric | Reaction | Aging | nm) of component | | | Yield |
| Example | Organic | solvent | acid | temperature | time | in reaction mixture | | | (%) |
| No. | solvent | (L/mol) | (mol %) | (° C.) | (h) | (7-a) | (9-a) | (8-a) | (8-a) |
| 2-7 | Toluene | 1.5 | 300 | 75 | 15 | 0 | 2.1 | 94.8 | 91 |
| 2-8 | Toluene | 1.5 | 200 | 75 | 6 | 0 | 3.1 | 94.9 | 86 |
| 2-9 | Toluene | 1.5 | 100 | 75 | 23 | 0 | 1.1 | 97.8 | 94 |
| 2-10 | Toluene | 1.5 | 100 | 100 | 20 | 0 | 3.0 | 92.4 | 86 |
| 2-11 | Butyl | 1.5 | 50 | 75 | 6 | 0 | 0 | 96.1 | 97 |
| | acetate | | | | | | | | |
| 2-12 | Butyl | 1.5 | 50 | 100 | 2 | 0 | 0 | 96.0 | 96 |
| | acetate | | | | | | | | |
| 2-13 | Butyl | 1.5 | 100 | 75 | 3 | 0 | 0 | 95.9 | 96 |
| | acetate | | | | | | | | |
| 2-14 | Butyl | 1.5 | 200 | 75 | 3 | 0 | 0 | 94.3 | 90 |
| | acetate | | | | | | | | |
| 2-15 | Butyl | 1.5 | 200 | 40 | 7 | 0 | 1.2 | 94.8 | 95 |
| | acetate | | | | | | | | |
| 2-16 | Butyl | 1.5 | 300 | 75 | 3 | 0 | 0 | 93.1 | 96 |
| | acetate | | | | | | | | |
| 2-17 | DMF | 1.5 | 300 | 75 | 6 | 0 | 3.6 | 91.4 | 97 |
| 2-18 | NMP | 1.5 | 300 | 75 | 6 | 0 | 0.7 | 89.7 | 91 |
| Comparative | Methanol | 1.5 | 100 | 66 | 24 | 0 | 40.1 | 45.0 | — |
| Example 4 | | | | | | | | | |
| Comparative | Methanol | 1.5 | 300 | 66 | 14 | 0 | 3.9 | 77.5 | 58 |
| Example 5 | | | | | | | | | |
| Comparative | Ethanol | 1.5 | 300 | 75 | 17 | 0 | 1.9 | 83.8 | 67 |
| Example 6 | | | | | | | | | |
| Comparative | Butanol | 1.5 | 300 | 75 | 24 | 26.9 | 35.3 | 3.8 | — |
| Example 7 | | | | | | | | | |

It was revealed that the (C1-C4)alcohol solvent, which was expected favorable based on prior art, was not favorable contrary to the expectation in the process using sulfuric acid described in Examples 2-1 to 2-18. On the other hand, when a reaction system is separated into two layers, reactivity is generally expected to be lowered. Even when a non-polar solvent such as toluene, which was expected to be separated from an aqueous hydrogen peroxide solution, was used, however, it has been found that the reaction sufficiently proceeds in employing this process using sulfuric acid. Aromatic hydrocarbon derivatives such as toluene are inexpensive, easily recycled, and contributes to sustainability. A wide range of organic solvents other than an alcohol can be used in this process, and it has been found that this process is versatile in solvents other than an alcohol. In other words, in one embodiment, this reaction using sulfuric acid can be performed in the presence of an organic solvent having a relative permittivity of 1 to 40 other than an alcohol. In another embodiment, this reaction can be performed in the presence of an organic solvent having an acceptor number of 5 to 25 and a relative permittivity of 1 to 40. In still another embodiment, this reaction can be performed in the presence of an organic solvent having a Rohrschneider's polarity parameter of 1 to 7 other than an alcohol. In still another embodiment, this reaction can be performed in the presence of an organic solvent having an acceptor number of 5 to 25 and a Rohrschneider's polarity parameter of 1 to 7. Herein, regarding the acceptor number, for example, the following document can be referred to: Christian Reichardt, “Solvents and Solvent Effects in Organic Chemistry”, 3rd, updated and enlarged edition, WILEY-VCH, 2003, p. 25-26. Herein, regarding the relative permittivity (generally known also as “dielectric constant”), for example, the following document can be referred to: “Handbook of Chemistry (Pure Chemistry)”, Maruzen Co., Ltd., 5th revised edition, 2004, p. I-770-777, edited by the Chemical Society of Japan. Regarding the Rohrschneider's polarity parameter, for example, the following website can be referred to: https://www.shodex.com/ja/dc/06/0117.html.

### Example 2-19

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 2.94 g (1.5 L/mol) of acetonitrile, trifluoroacetic acid (0.86 g, 7.50 mmol, 300 mol %), and a 30% aqueous hydrogen peroxide solution (0.81 g, 7.12 mmol, 285 mol %, containing 0.57 g (0.2 L/mol) of water) were added to a reaction flask, followed by stirring at 75° C. for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 89%.

### Example 2-20

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 2.97 g (1.5 L/mol) of methanol, trifluoroacetic acid (0.86 g, 7.50 mmol, 300 mol %), and a 30% aqueous hydrogen peroxide solution (0.81 g, 7.12 mmol, 285 mol %, containing 0.57 g (0.2 L/mol) of water) were added to a reaction flask, followed by stirring at 75° C. for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0.8% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 90%.

### Example 2-22

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 3.93 g (1.5 L/mol) of acetic acid, sulfuric acid (0.25 g, 2.5 mmol, 100 mol %), and a 35% aqueous hydrogen peroxide solution (0.69 g, 7.12 mmol, 285 mol %, containing 0.45 g (0.18 L/mol) of water) were added to a reaction flask, followed by stirring at 75° C. for aging for 48 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 2.4% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 88.7%.

### Examples 2-23 to 2-28

The reaction and the analysis were performed in the same manner as in Example 2-22 except that the acid, the amount thereof, the reaction temperature and the aging time were changed as shown in Table 4. The results are shown in Table 4. In addition, the results of Example 2-22 are also summarized in Table 4.

TABLE 4

| | Amount of | | | | | HPLC area % (230 | | | |
|---|---|---|---|---|---|---|---|---|---|
| | acetic acid | | Amount | Reaction | Aging | nm) of component | | | Yield |
| Example | solvent | | of acid | temperature | time | in reaction mixture | | | (%) |
| No. | (L/mol) | Acid | (mol %) | (° C.) | (h) | (7-a) | (9-a) | (8-a) | (8-a) |
| 2-22 | 1.5 | Sulfuric acid | 100 | 25-30 | 48 | 0 | 2.4 | 94.0 | 89 |
| 2-23 | 1.5 | Sulfuric acid | 200 | 25-30 | 48 | 0 | 2.6 | 93.6 | 82 |
| 2-24 | 1.5 | Sulfuric acid | 300 | 50 | 12 | 0 | 0.7 | 93.1 | 89 |
| 2-25 | 1.5 | Sulfuric acid | 300 | r.t. | 28 | 0 | 2.2 | 92.9 | 91 |
| 2-26 | 1.5 | Trifluoroacetic acid | 300 | 50 | 6 | 0 | 1.8 | 93.1 | 93 |
| 2-27 | 1.5 | Trifluoroacetic acid | 300 | r.t. | 28 | 0 | 2.0 | 94.2 | 95 |
| 2-28 | 1.5 | — | — | 50 | 12 | 0 | 1.3 | 95.1 | 94 |

### Example 2-29

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 2.94 g (1.5 L/mol) of acetonitrile, potassium hydrogen sulfate (1.02 g, 7.50 mmol, 300 mol %), and a 30% aqueous hydrogen peroxide solution (0.81 g, 7.12 mmol, 285 mol %, containing 0.57 g (0.2 L/mol) of water) were added to a reaction flask, followed by stirring at 75° C. for aging for 48 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 1.3% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 88%.

As understood from Examples described above, the acidic compound, particularly sulfuric acid, may be a salt. The process for performing the reaction in the step ii in the presence of a sulfuric acid salt is within the scope of the present invention.

### Example 2-30

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 0.99 g (0.5 L/mol) of acetonitrile, acetic acid (2.25 g, 37.5 mmol, 1500 mol %, 0.86 L/mol), and a 35% aqueous hydrogen peroxide solution (0.69 g, 7.12 mmol, 285 mol %, containing 0.45 g (0.18 L/mol) of water) were added to a reaction flask, followed by stirring at 50° C. for aging for 24 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 3.38% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 90%.

### Example 2-31

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (8.98 g, purity: 100%, 25.0 mmol, 100 mol %), 29.6 g (1.5 L/mol) of acetonitrile, sulfuric acid (7.51 g, 75.0 mmol, 300 mol %), and a 35% aqueous hydrogen peroxide solution (6.92 g, 71.3 mmol, 285 mol %, containing 4.50 g (0.18 L/mol) of water) were mixed in a flask in an ice bath. The whole amount of the resultant mixture was filled in a syringe, and transferred with a syringe pump at 0.2 mL/min. The transferred mixture passed through a Teflon tube having an internal diameter of 2.4 mm and a length of 15 m, and submerged in an oil bath at 80° C. to be accumulated in another flask. At a time point two hours after starting the transfer, the reaction mixture was collected and analyzed, and it was thus found that 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0.57% (HPLC area percentage; 230 nm).

The target product (8-a) was 90% (HPLC area percentage; 230 nm).

The transfer was further continued, and at a time point after 4 hours, the reaction solution was collected and analyzed, and it was thus found that 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

The target product (8-a) was 95% (HPLC area percentage; 230 nm).

### Example 2-32

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (3.59 g, purity: 100%, 10.0 mmol, 100 mol %), 7.88 g (1.0 L/mol) of acetonitrile, trifluoroacetic acid (3.42 g, 30.0 mmol, 300 mol %), and a 35% aqueous hydrogen peroxide solution (2.77 g, 28.5 mmol, 285 mol %, containing 1.80 g (0.18 L/mol) of water) were mixed in a flask at room temperature. The resultant mixture was transferred with a plunger pump at 0.1 mL/min. The transferred mixture passed through a tube having an internal diameter of 4 mm and a length of 3.6 mm, and submerged in a hot water bath at 90° C. to be accumulated in another flask. At a time point two hours after starting the transfer, the reaction mixture was collected and analyzed, and it was thus found that 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

The target product (8-a) was 91% (HPLC area percentage; 230 nm).

### Example 3-1

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 3.14 g (1.6 L/mol) of acetonitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 2 ml of a 0.6 M aqueous potassium carbonate solution (0.8 L/mol, 48 mol %) was added, followed by aging at room temperature for 30 minutes.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 88%.

### Example 3-2

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 4.0 g (1.6 L/mol) of benzonitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 2 ml of a 0.6 M aqueous potassium carbonate solution (0.8 L/mol, 48 mol %) was added, followed by aging at room temperature for 17 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC, the target product (8-a) was obtained with a yield of 87.0% (HPLC area percentage; 230 nm).

### Example 3-3

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 3.08 g (1.6 L/mol) of isobutyronitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 2 ml of a 0.6 M aqueous potassium carbonate solution (0.8 L/mol, 48 mol %) was added, followed by aging at room temperature for 16 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC, the target product (8-a) was obtained with a yield of 95.6% (HPLC area percentage; 230 nm).

### Example 3-4

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 3.78 g (1.6 L/mol) of dimethylformamide, succinonitrile (0.50 g, 12.5 mmol, 250 mol %), and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 2 ml of a 0.6 M aqueous potassium carbonate solution (0.8 L/mol, 48 mol %) was added, followed by aging at room temperature for 18 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0.9% (HPLC area percentage; 230 nm).

Acetonitrile was added to the reaction mixture to dissolve the reaction mixture in a homogeneous solution. As a result of analysis by the HPLC, the target product (8-a) was obtained with a yield of 89.7% (HPLC area percentage; 230 nm).

### Example 3-5

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 3.78 g (1.6 L/mol) of dimethylformamide, p-nitrobenzonitrile (1.85 g, 12.5 mmol, 500 mol %), and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 2 ml of a 0.6 M aqueous potassium carbonate solution (0.8 L/mol, 48 mol %) was added, followed by aging at room temperature for 30 minutes.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0.3% (HPLC area percentage; 230 nm).

Acetonitrile was added to the reaction mixture to dissolve the reaction mixture in a homogeneous solution. As a result of analysis by the HPLC, the target product (8-a) was obtained with a yield of 87.2% (HPLC area percentage; 230 nm).

### Example 3-6

Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 3.14 g (1.6 L/mol) of acetonitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 6 ml of a 0.6 M aqueous potassium carbonate solution (2.4 L/mol, 144 mol %) was added, followed by aging for 18 hours. At this point of time, the pH was 8.25.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution.

As a result of analysis by the HPLC external standard method, the target product (8-a) was obtained with a yield of 94%.

### Example 3-7

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 3.14 g (1.6 L/mol) of acetonitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 2 ml of a 0.6 M aqueous sodium carbonate solution (0.8 L/mol, 48 mol %) was added, followed by aging for 2 hours. At this point of time, the pH was 7.85.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 89%.

### Example 3-8

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 3.14 g (1.6 L/mol) of acetonitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 6 ml of a 0.6 M aqueous sodium hydrogen carbonate solution (2.4 L/mol, 144 mol %) was added, followed by aging for 18 hours. At this point of time, the pH was 7.98.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 96%.

### Example 3-9

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %) was dissolved in 3.14 g (1.6 L/mol) of acetonitrile in a reaction flask, followed by stirring at a temperature of 50 to 60° C. To the resultant, 2 ml of a 0.6 M aqueous potassium carbonate solution (0.8 L/mol, 48 mol %) and a 35% aqueous hydrogen peroxide solution (1.22 g, 12.5 mmol, 500 mol %, containing 0.79 g (0.3 L/mol) of water) were simultaneously added dropwise over 5 hours, followed by stirring at 60° C. for aging for 1 hour.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 0.39% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 82%.

### Examples 3-10 to 3-16

The reaction and the analysis were performed in the same manner as in Example 3-9 except that the amount of the hydrogen peroxide, the dropwise addition time of the hydrogen peroxide, the base, the amount of the base, the reaction temperature and the aging time were changed as shown in Table 5. The reaction temperature means a dropwise addition time and an aging time. The results are shown in Table 5. In addition, the results of Example 3-9 are also summarized in Table 5.

The addition rate of the base of the hydrogen peroxide in Examples 3-9 to 3-10 was 0.1 mol/hr. or 0.5 mol/hr. based on 1 mol of the compound of the formula (7).

The addition rate of the hydrogen peroxide in Examples 3-9 to 3-10 was 1 mol/hr. or 5 mol/hr. based on 1 mol of the compound of the formula (7).

TABLE 5

| | | | | | Dropwise | | HPLC area % (230 | | | |
|---|---|---|---|---|---|---|---|---|---|---|
| | Hydrogen | | Amount | Reaction | addition | Aging | nm) of component | | | Yield |
| Example | peroxide | | of base | temperature | time | time | in reaction mixture | | | (%) |
| No. | (mol %) | Base | (mol %) | (° C.) | (h) | (h) | (7-a) | (9-a) | (8-a) | (8-a) |
| 3-9 | 500 | Potassium | 48 | 60-70 | 5 | 1 | 0 | 0.4 | 92.4 | 82 |
| | | carbonate | | | | | | | | |
| 3-10 | 500 | Potassium | 48 | 60-70 | 1 | 1 | 0 | 0 | 94.0 | 91 |
| | | carbonate | | | | | | | | |
| 3-11 | 350 | Sodium | 28 | 60-70 | 1 | 1 | 0 | 0 | 91.4 | 82 |
| | | carbonate | | | | | | | | |
| 3-12 | 500 | Sodium | 48 | 60-70 | 1 | 1 | 0 | 0 | 90.6 | 80 |
| | | carbonate | | | | | | | | |
| 3-13 | 350 | Potassium | 36 | 50-60 | 1 | 1 | 0 | 0 | 94.0 | 90 |
| | | hydrogen | | | | | | | | |
| | | carbonate | | | | | | | | |
| 3-14 | 500 | Potassium | 48 | 50-60 | 1 | 1 | 0 | 0 | 92.7 | 89 |
| | | hydrogen | | | | | | | | |
| | | carbonate | | | | | | | | |
| 3-15 | 350 | Sodium | 36 | 50-60 | 1 | 1 | 0 | 0 | 94.1 | 91 |
| | | hydrogen | | | | | | | | |
| | | carbonate | | | | | | | | |
| 3-16 | 500 | Sodium | 48 | 50-60 | 1 | 1 | 0 | 0 | 92.5 | 88 |
| | | hydrogen | | | | | | | | |
| | | carbonate | | | | | | | | |

### Example 4

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Under a nitrogen stream, the compound (7-a) (0.90 g, purity: 100%, 2.5 mmol, 100 mol %), 2.94 g (1.5 L/mol) of acetonitrile, sulfuric acid (0.023 g, 0.225 mmol, 9 mol %) and a 30% aqueous hydrogen peroxide solution (0.81 g, 7.12 mmol, 285 mol %, containing 0.57 g (0.2 L/mol) of water) were added to a reaction flask, followed by stirring at 75° C. for aging for 6 hours.

Thereafter, the resultant reaction mixture was cooled to room temperature, and at this point of time, the pH was −0.05.

While the reaction mixture was being stirred at room temperature, a 30% aqueous hydrogen peroxide solution (0.61 g, 5.37 mmol, 215 mol %, containing 0.43 g (0.17 L/mol) of water), and a 0.6 M aqueous potassium carbonate solution (3.0 g, 1.80 mmol, 72 mol %) were added thereto, followed by stirring at room temperature for aging for 0.5 hours. At this point of time, the pH was 9.31.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 1.51% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 80%.

### Example 5-1

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.45 g, purity: 100%, 1.25 mmol, 100 mol %), 0.83 g (0.85 L/mol) of acetonitrile, 3.19 g (2.55 L/mol) of water, and 45% potassium hydrogen persulfate (1.88 g, 1.38 mmol, 110 mol %) were added to a reaction flask, followed by stirring at 80° C. for aging for 3 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 3.12% (HPLC area percentage; 230 nm).

At this point of time, the target product (8-a) was 95.7% (HPLC area percentage; 230 nm).

### Example 5-2

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.45 g, purity: 100%, 1.25 mmol, 100 mol %), 0.83 g (0.85 L/mol) of acetonitrile, 3.19 g (2.55 L/mol) of water, 45% potassium hydrogen persulfate (1.88 g, 1.38 mmol, 110 mol %), and cyclohexane (0.04 g, 0.25 mmol, 20 mol %) were added to a reaction flask, followed by stirring at 80° C. for aging for 3 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 4.13% (HPLC area percentage; 230 nm).

At this point of time, the target product (8-a) was 94.4% (HPLC area percentage; 230 nm).

### Reference Example 2

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 2-1.

Process described in Example 4 of CN 111574511 A (Patent Document 10)

Under a nitrogen stream, the compound (7-a) (0.90 g, purity: 100%, 2.5 mmol, 100 mol %), 2.97 g (1.5 L/mol) of methanol, sulfuric acid (0.023 g, 0.225 mmol, 9 mol %) and a 30% aqueous hydrogen peroxide solution (0.81 g, 7.12 mmol, 285 mol %, containing 0.57 g (0.2 L/mol) of water) were added to a reaction flask, followed by stirring at room temperature for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 13.97% (HPLC area percentage; 230 nm).

Acetonitrile was added to the reaction mixture to dissolve the reaction mixture in a homogeneous solution. As a result of analysis by the HPLC external standard method, the yield was 0%, and the target product (8-a) was not obtained. This process is not reproducible.

### Reference Example 3

### Production of 3-[(5-difluoromethoxy-1-methyl-3-trifluoromethylpyrazol-4-yl)methylsulfonyl]-4,5-dihydro-5,5-dimethylisoxazole

The reaction was performed in the same manner as in the process described in Example 4 of CN 111574511 A (Patent Document 10) except that the reaction temperature was changed to heating conditions.

The reaction formulas are the same as those of Example 2-1.

Under a nitrogen stream, the compound (7-a) (0.90 g, purity: 100%, 2.5 mmol, 100 mol %), 2.97 g (1.5 L/mol) of methanol, sulfuric acid (0.023 g, 0.225 mmol, 9 mol %) and a 30% aqueous hydrogen peroxide solution (0.81 g, 7.12 mmol, 285 mol %, containing 0.57 g (0.2 L/mol) of water) were added to a reaction flask, followed by stirring at 66° C. for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 93.8% (HPLC area percentage; 230 nm).

Acetonitrile was added to the reaction mixture to dissolve the reaction mixture in a homogeneous solution. As a result of analysis by the HPLC external standard method, the target product (8-a) was obtained with a yield of 4.4%. The yield was thus very low.

### Comparative Example 9

(Examination of Acidic Compound)

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 2.94 g (1.5 L/mol) of acetonitrile, benzoic acid (0.92 g, 7.50 mmol, 300 mol %), and a 35% aqueous hydrogen peroxide solution (0.69 g, 7.12 mmol, 285 mol %, containing 0.45 g (0.18 L/mol) of water) were added to a reaction flask, followed by stirring at 75° C. for aging for 24 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 80.92% (HPLC area percentage; 230 nm).

Acetonitrile was added to the reaction mixture to dissolve the reaction mixture in a homogeneous solution. As a result of analysis by HPLC analysis (area percentage; 230 nm), the target product (8-a) was 17%.

### Comparative Examples 10 to 16

(Examination of Acidic Compound)

The reaction and the analysis were performed in the same manner as in Comparative Example 9 except that the acid, the equivalents of the acid, the reaction temperature and the aging time were changed as shown in Table 6. The results are shown in Table 6. In addition, the results of Comparative Example 9 are also summarized in Table 6.

TABLE 6

| | | | | | HPLC area % (230 | | | |
|---|---|---|---|---|---|---|---|---|
| | | Amount | Reaction | Aging | nm) of component | | | Yield |
| Comparative | | of acid | temperature | time | in reaction mixture | | | (%) |
| Example No. | Acid | (mol %) | (° C.) | (h) | (7-a) | (9-a) | (8-a) | (8-a) |
| 9 | Benzoic | 300 | 75 | 24 | 0 | 80.9 | 16.7 | — |
| | acid | | | | | | | |
| 10 | Hexanoic | 300 | 75 | 6 | 58.8 | 37.2 | 0.4 | — |
| | acid | | | | | | | |
| 11 | Fumaric | 300 | 75 | 24 | 2.2 | 93.2 | 2.7 | — |
| | acid | | | | | | | |
| 12 | Phosphoric | 300 | 75 | 38 | 0 | 2.1 | 86.0 | 73 |
| | acid | | | | | | | |
| 13 | Formic acid | 300 | 50 | 24 | 0 | 87.6 | 9.6 | — |
| 14 | Oxalic acid | 300 | 75 | 24 | 0 | 85.8 | 9.5 | — |
| 15 | Acetic acid | 300 | 50 | 6 | 91.2 | 4.8 | 0 | — |
| 16 | Acetic acid | 1000 | 50 | 24 | 0 | 35.7 | 61.7 | — |

### Comparative Example 17

The reaction was performed in the same manner as in Example 5-1 except that potassium hydrogen persulfate in about 3-fold amount was used at first. Contrary to the expectation, the yield was low.

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The compound (7-a) (0.45 g, purity: 100%, 1.25 mmol, 100 mol %), 0.83 g (0.85 L/mol) of acetonitrile, 3.19 g (2.55 L/mol) of water, and 45% potassium hydrogen persulfate (5.12 g, 3.75 mmol, 300 mol %) were added to a reaction flask, followed by stirring at 80° C. for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 31.47% (HPLC area percentage; 230 nm).

At this point of time, the target product (8-a) was 61.98% (HPLC area percentage; 230 nm).

### Comparative Example 18

The reaction was performed in the same manner as in Example 5-2 except that potassium hydrogen persulfate in about 3-fold amount was used at first. Contrary to the expectation, the yield was low.

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.45 g, purity: 100%, 1.25 mmol, 100 mol %), 0.83 g (0.85 L/mol) of acetonitrile, 3.19 g (2.55 L/mol) of water, 45% potassium hydrogen persulfate (5.12 g, 3.75 mmol, 300 mol %), and cyclohexane (0.04 g, 0.25 mmol, 20 mol %) were added to a reaction flask, followed by stirring at 80° C. for aging for 7 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 29.6% (HPLC area percentage; 230 nm).

At this point of time, the target product (8-a) was 69.6% (HPLC area percentage; 230 nm).

### Comparative Examples 19 to 22

(Examination of Solvent in Using Potassium Hydrogen Persulfate)

The reaction was performed in the same manner as in Example 5-1 except for the solvent, the reaction temperature and the aging time. Contrary to the expectation, the yield was low in using any of the solvents. The results are shown in Table 7.

TABLE 7

| | | Reaction | Aging | HPLC area % (230 nm) of | | |
|---|---|---|---|---|---|---|
| Comparative | | temperature | time | component in reaction mixture | | |
| Example No. | Solvent | (° C.) | (h) | (7-a) | (9-a) | (8-a) |
| 19 | Water | 80 | 12 | 34.3 | 27.6 | 35.2 |
| 20 | Butyl acetate | 80 | 12 | 79.1 | 13.4 | 4.9 |
| 21 | Toluene | 80 | 12 | 65.3 | 16.6 | 5.6 |
| 22 | Dichloromethane | 40 | 12 | 45.1 | 49.5 | 2.5 |

### Comparative Example 23

(Examination of Base)

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %), 3.14 g (1.6 L/mol) of acetonitrile, and a 35% aqueous hydrogen peroxide solution (1.22 g, 12.5 mmol, 500 mol %, containing 0.79 g (0.3 L/mol) of water) were added to a reaction flask, followed by stirring at room temperature. To the resultant, 6 ml (2.4 L/mol) of a 0.6 M aqueous sodium acetate solution was added, followed by aging for 18 hours. At this point of time, the pH was 6.70.

At this point of time, 3-[(5-difluoromethoxy-1-methyl-3-trifluoromethylpyrazol-4-yl)methylsulfinyl]-4,5-dihydro-5,5-dimethylisoxazole (Compound 9-a; SO derivative), which is a reaction intermediate, was 3.7% (HPLC area percentage; 230 nm).

Acetonitrile was added to the reaction mixture to dissolve the reaction mixture in a homogeneous solution. As a result of analysis by HPLC analysis (area percentage; 230 nm), the target product (8-a) was 20%.

### Comparative Examples 24 to 31

(Examination of Base and Examination of Solvent in the Presence of Base)

The reaction and the analysis were performed in the same manner as in Comparative Example 23 except that the solvent, the amount of the solvent, the base, the equivalents of the base, the reaction temperature and the aging time were changed as shown in Table 8. The results are shown in Table 8. In addition, the results of Comparative Example 23 are also summarized in Table 8.

TABLE 8

| | | | | | | | | | HPLC area % (230 | | |
|---|---|---|---|---|---|---|---|---|---|---|---|
| | | Amount of | | Amount of | | Amount | Reaction | Aging | nm) of component in | | |
| Comparative | | solvent | | additive | | of base | temperature | time | reaction mixture | | |
| Example No. | Solvent | (L/mol) | Additive | (mol %) | Base | (mol %) | (° C.) | (h) | (7-a) | (9-a) | (8-a) |
| 23 | Acetonitrile | 1 | — | — | Sodium | 144 | r.t. | 18 | 73.2 | 3.7 | 20.0 |
| | | | | | acetate | | | | | | |
| 24 | Toluene | 1.6 | Acetonitrile | 500 | 0.6M | 48 | r.t. | 0.5 | | | trace |
| | | | | | K2CO3 | | | | | | |
| 25 | Toluene | 0.8 | Acetonitrile | 1530 | 0.6M | 48 | r.t. | 0.5 | 62.2 | 4.5 | 29.3 |
| | | | | | K2CO3 | | | | | | |
| 26 | DMF | 1.6 | Acetonitrile | 500 | 0.6M | 48 | r.t. | 0.5 | 66.6 | 3.9 | 19.8 |
| | | | | | K2CO3 | | | | | | |
| 27 | Water | 1.6 | Acetonitrile | 500 | 0.6M | 48 | r.t. | 0.5 | 79.4 | 4.3 | 13.4 |
| | | | | | K2CO3 | | | | | | |
| 28 | MeOH | 1.6 | Acetonitrile | 300 | 0.6M | 48 | r.t. | 12 | 73.1 | 4.8 | 6.8 |
| | | | | | K2CO3 | | | | | | |
| 29 | Acetonitrile | 1 | — | — | 25% NaOH | 144 | r.t. | 18 | 30.7 | 1.6 | 65.9 |
| 30 | Acetonitrile | 1 | — | — | Sodium | 144 | r.t. | 18 | 29.7 | 1.7 | 64.0 |
| | | | | | borate | | | | | | |
| 31 | Acetonitrile | 1 | — | — | Sodium | 144 | r.t. | 18 | 73.2 | 3.7 | 20.0 |
| | | | | | phosphate | | | | | | |

### Comparative Example 32

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %) was dissolved in 3.14 g (1.6 L/mol) of acetonitrile in a reaction flask, followed by stirring at a temperature of 50 to 60° C. To the resultant, 2 ml (0.8 L/mol, 48 mol %) of a 0.6 M aqueous potassium carbonate solution and a 35% aqueous hydrogen peroxide solution (1.22 g, 12.5 mmol, 500 mol %, containing 0.79 g (0.3 L/mol) of water) were simultaneously added dropwise over 30 minutes, followed by aging at 60° C. for 2 hours.

The compound (7-a), which is a raw material, was 9.6% (HPLC area percentage; 230 nm), and a reaction intermediate (Compound 9-a; SO derivative) was 0.6% (HPLC area percentage; 230 nm).

The target product (8-a) was 84.7% (HPLC area percentage; 230 nm).

When the addition time was short, the yield was comparatively low.

### Comparative Example 33

### Production of 3-[(5-difluoromethoxy-1-methyl-3-trifluoromethylpyrazol-4-yl)methylsulfonyl]-4,5-dihydro-5,5-dimethylisoxazole (Compound 8-a)

The reaction formulas are the same as those of Example 2-1.

The compound (7-a) (0.90 g, purity: 100%, 2.5 mmol, 100 mol %) was dissolved in 3.14 g (1.6 L/mol) of acetonitrile in a reaction flask, and 2 ml (0.8 L/mol, 48 mol %) of a 0.6 M aqueous potassium carbonate solution was added thereto, followed by stirring at a temperature of 50 to 60° C. To the resultant, a 35% aqueous hydrogen peroxide solution (1.22 g, 12.5 mmol, 500 mol %, containing 0.79 g (0.3 L/mol) of water) was added dropwise over 30 minutes, followed by aging at 60° C. for 2 hours.

The compound (7-a), which is a raw material, was 82.0% (HPLC area percentage; 230 nm), and a reaction intermediate (Compound 9-a; SO derivative) was 3.8% (HPLC area percentage; 230 nm).

The target product (8-a) was 11.1% (HPLC area percentage; 230 nm).

When only the base was added dropwise after adding the hydrogen peroxide, the yield was further low.

### Comparative Example 34

### Production of 3-[(1,3,5-trimethylpyrazol-4-yl)methylsulfonyl]-4,5-dihydro-5,5-dimethylisoxazole

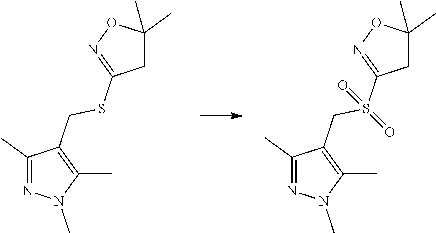

The compound (0.21 g, purity: 100%, 0.83 mmol, 100 mol %), 0.98 g (1.5 L/mol) of acetonitrile, sulfuric acid (0.25 g, 2.50 mmol, 300 mol %), and a 30% aqueous hydrogen peroxide solution (0.27 g, 2.37 mmol, 285 mol %, containing 0.19 g (0.22 L/mol of water)) were added to a reaction flask, followed by stirring at 75° C. for aging for 6 hours.

Acetonitrile was added to the reaction mixture to dissolve the reaction mixture in a homogeneous solution. As a result of analysis by HPLC analysis (area percentage; 230 nm), the target product was 0.5%.

### Comparative Examples 35 to 37

The reaction and the analysis were performed in the same manner as in Comparative Example 34 except that the substituents in the raw material, the solvent and the acid were changed as shown in Table 9. The results are shown in Table 9.

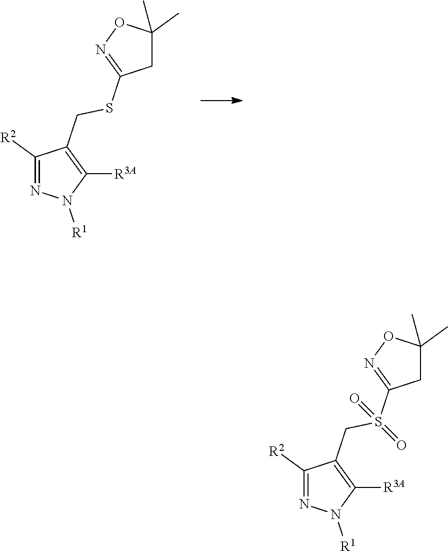

wherein R^{3A} is as shown in Table 9.

TABLE 9

| | | | | | | Amount | Reaction | Aging | HPLC area % |
|---|---|---|---|---|---|---|---|---|---|
| Comparative | | | | | | of acid | temperature | time | (230 nm) |
| Example No. | R1 | R2 | R3A | Solvent | Acid | (mol %) | (° C.) | (h) | (9-a) |
| 34 | Me | Me | Me | Acetonitrile | Sulfuric | 300 | 75 | 6 | 0.5 |
| | | | | | acid | | | | |
| 35 | Me | CF3 | H | Acetonitrile | Sulfuric | 300 | 75 | 6 | 0 |
| | | | | | acid | | | | |
| 36 | Me | Me | Me | Acetic acid | — | — | 50 | 12 | 0.4 |
| 37 | Me | CF3 | H | Acetic acid | — | — | 50 | 12 | 0 |

### Comparative Example 38

(Step iii)

### Production of 3-[(5-hydroxy-1-methyl-3-trifluoromethylpyrazol-4-yl)methylsulfonyl]-4,5-dihydro-5,5-dimethylisoxazole

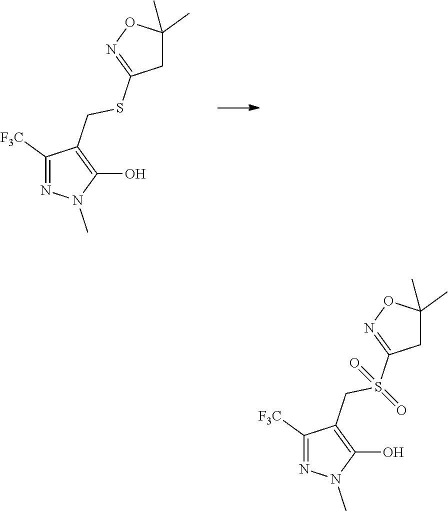 [SMILES: Cn1nc(C(F)(F)F)c(CSC2=NOC(C)(C)C2)c1O ; Cn1nc(C(F)(F)F)c(CS(=O)(=O)C2=NOC(C)(C)C2)c1O]

The compound (0.26 g, purity: 100%, 0.83 mmol, 100 mol %), 1.05 g (1.6 L/mol) of acetonitrile, and a 35% aqueous hydrogen peroxide solution (0.41 g, 14.17 mmol, 500 mol %, containing 0.28 q (0.34 L/mol) of water) were added to a reaction flask, followed by stirring at room temperature. To the resultant, 0.67 ml (0.8 L/mol) of a 0.6 M aqueous potassium carbonate solution was added, followed by aging for 30 minutes.

Acetonitrile was added to the reaction mixture to dissolve the reaction mixture in a homogeneous solution. At this point of time, the target product was 0% (HPLC area percentage; 230 nm).

### Comparative Example 39

The reaction and the analysis were performed in the same manner as in Comparative Example 36 except that the substituents in the raw material were changed as shown in Table 10. The results are shown in Table 10. In addition, the results of Comparative Example 36 are also summarized in Table 10.

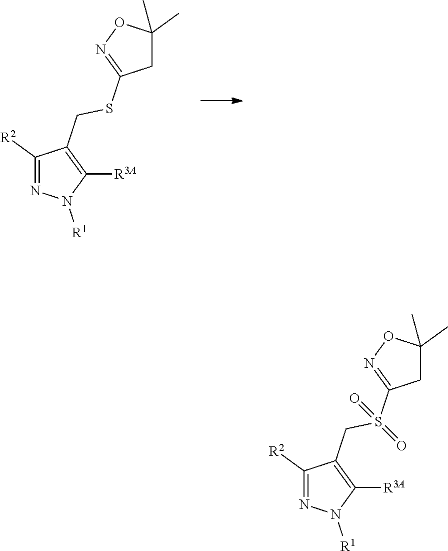

wherein R^{3A} is as shown in Table 10.

TABLE 10

| | | | | | | Amount | Reaction | Aging | HPLC area % |
|---|---|---|---|---|---|---|---|---|---|
| Comparative | | | | | | of acid | temperature | time | (230 nm) |
| Example No. | R1 | R2 | R3A | Solvent | Acid | (mol %) | (° C.) | (h) | (9-a) |
| 38 | Me | CF3 | H | Acetonitrile | Sulfuric | 300 | 75 | 6 | 0 |
| | | | | | acid | | | | |
| 39 | Me | Me | Me | Acetonitrile | Sulfuric | 300 | 75 | 6 | 0.5 |
| | | | | | acid | | | | |

All publications, patents, and patent applications described herein are hereby fully incorporated by reference in their entirety for the purpose of describinq and disclosing the methods described in those publications, patents, and patent applications that may be used in connection with the description herein. To the extent necessary to understand or complete the disclosure of the present invention, all publications, patents, and patent applications described herein are expressly incorporated herein by reference to the same extent as if each were individually incorporated. All publications, patents, and patent applications discussed above and throughout this specification are provided solely for disclosure prior to the filing date of this application.

Any processes and reagents similar or equivalent to those described herein can be employed in the practice of the present invention. Accordingly, the present invention is not to be limited by the foregoing description, but is intended to be defined by the claims and their equivalents. Those equivalents fall within the scope of the present invention as defined by the appended claims.

### INDUSTRIAL APPLICABILITY

As disclosed in Patent Document 1, a compound of the general formula (8) (sulfone derivative: SO₂ derivative) has excellent herbicidal activity. According to the present invention, an industrially favorable novel production process for the compound of the general formula (8) useful as a herbicide is provided.

As described above herein, the process of the present invention is economical, is environmentally friendly, and is highly industrially variable. In particular, in the process of the present invention, the ratio of a compound of the formula (9) (sulfoxide derivative: SO derivative) in a product is sufficiently low. Here, the compound of the formula (9) (sulfoxide derivative: SO derivative) is an intermediate of an oxidation reaction, and can be a cause of reduced quality as a herbicide and crop injury. In addition, a reproducible and practicable process has been provided by the present invention. Accordingly, the present invention is highly industrially applicable.

1. A process for producing a compound of formula (8), comprising the following step ii:
(step ii) reacting a compound of formula (7) with an oxidizing agent in the absence of a transition metal and in the presence of a base to produce the compound of formula (8): 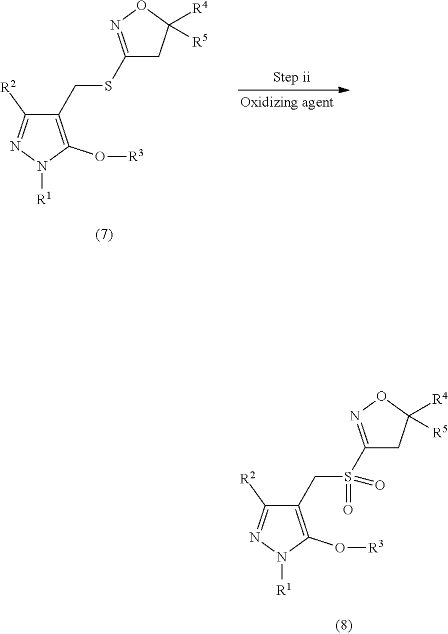
wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.

2. The process according to claim 1, wherein the reaction in the step ii is performed in the presence of an organic solvent, and the organic solvent is an organic solvent other than an alcohol.

3. The process according to claim 1, wherein the organic solvent is acetonitrile.

4. The process according claim 1, comprising simultaneously adding the base in the step ii and the oxidizing agent in the step ii.

5. The process according to claim 1, wherein the base in the step ii is selected from sodium hydrogen carbonate, potassium hydrogen carbonate, sodium carbonate and potassium carbonate.

6. The process according to claim 1, wherein the oxidizing agent in the step ii is hydrogen peroxide.

7. A process for producing a compound of formula (8), comprising the following step ii:
(step ii) reacting a compound of formula (7) with an oxidizing agent in the absence of a transition metal and in the presence of an acidic compound to produce the compound of the formula (8), wherein the acidic compound is sulfuric acid: 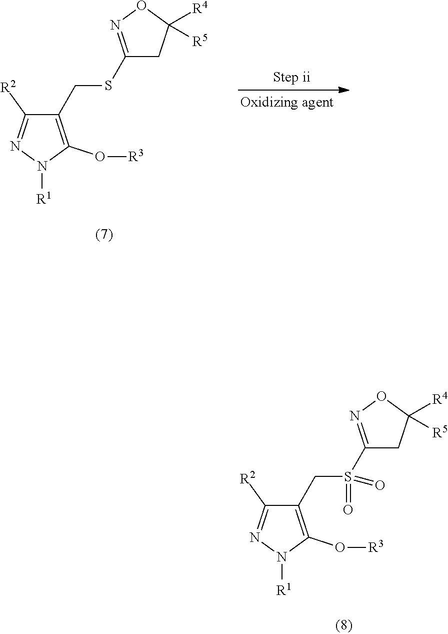
wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.

8. The process according to claim 7, wherein the reaction in the step ii is performed in the presence of an organic solvent having an acceptor number of 5 to 25 and a relative permittivity of 1 to 40.

9. The process according to claim 7, wherein the reaction in the step ii is performed in the presence of an organic solvent having an acceptor number of 5 to 25 and a Rohrschneider's polarity parameter of 1 to 7.

10. The process according to claim 8, wherein the organic solvent is an organic solvent other than an alcohol.

11. The process according to claim 8, wherein the organic solvent is selected from aromatic hydrocarbon derivatives, nitriles, carboxylic acid esters, and amides.

12. The process according to claim 7, wherein the oxidizing agent in the step ii is hydrogen peroxide.

13. A process for producing a compound of formula (8), comprising the following step ii:
(step ii) reacting a compound of formula (7) with an oxidizing agent in the absence of a transition metal and in the presence of an acidic compound to produce the compound of the formula (8), wherein the acidic compound is a (C2-C4)alkanoic acid substituted with 1 to 7 fluorine atoms: 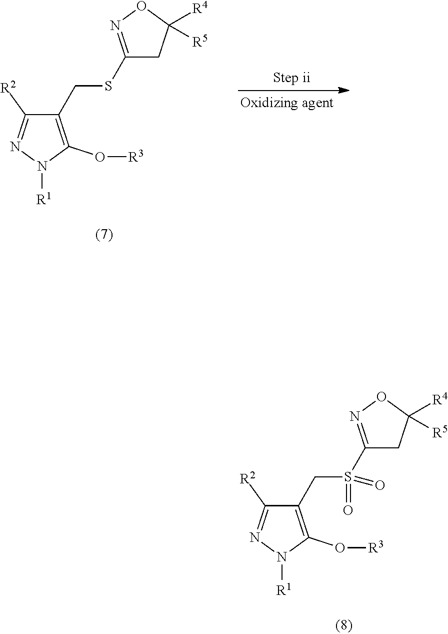
wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.

14. The process according to claim 13, wherein the (C2-C4)alkanoic acid substituted with 1 to 7 fluorine atoms is trifluoroacetic acid.

15. The process according to claim 13, wherein the oxidizing agent in the step ii is hydrogen peroxide.

16. A process for producing a compound of formula (8), comprising the following step ii:
(step ii) reacting a compound of formula (7) with an oxidizing agent in the absence of a transition metal and in the presence of an organic solvent to produce the compound of the formula (8), wherein the organic solvent is a (C1-C4)alkanoic acid: 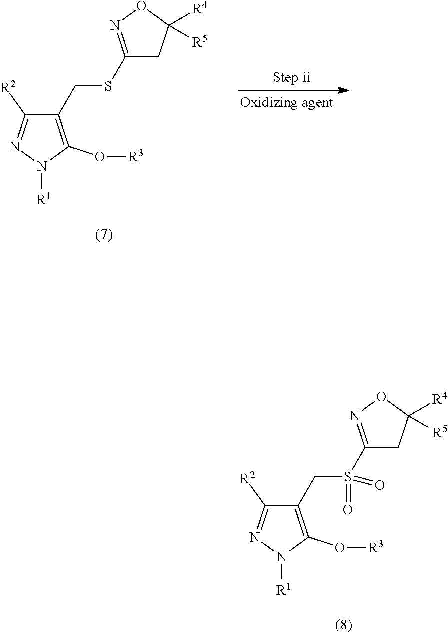
wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.

17. The process according to claim 16, wherein the (C1-C4)alkanoic acid is acetic acid.

18. The process according to claim 16, wherein the oxidizing agent in the step ii is hydrogen peroxide.

19. A process for producing a compound of formula (8), comprising the following step ii:
(step ii) reacting a compound of formula (7) with an oxidizing agent in the absence of a transition metal to produce the compound of the formula (8), wherein the oxidizing agent is an alkali metal persulfate, an ammonium persulfate salt, or an alkali metal hydrogen persulfate: 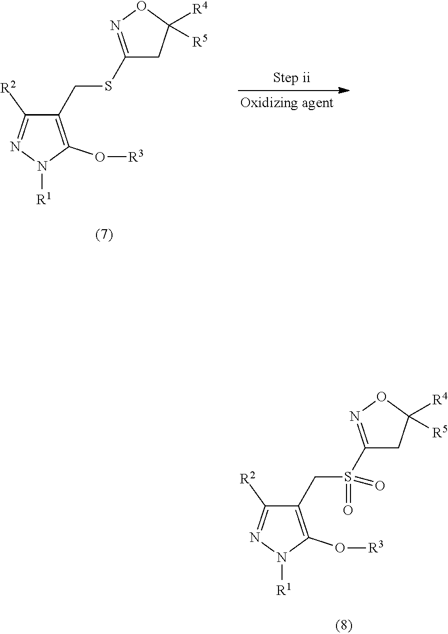
wherein in the formula (7) and the formula (8),
R¹, R², and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents, or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.

20. The process according to claim 19, wherein the oxidizing agent is sodium hydrogen persulfate, potassium hydrogen persulfate, potassium persulfate, sodium persulfate, or ammonium persulfate.

21. The process according to claim 20, wherein the reaction in the step ii is performed in the presence of an organic solvent, and the organic solvent is acetonitrile.

22. The process according to claim 1,
wherein in the formula (7) and the formula (8),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

23. The process according to claim 1,
wherein in the formula (7) and the formula (8),
R¹ is methyl,
R² is trifluoromethyl,
R³ is difluoromethyl, and
R⁴ and R⁵ are methyl.

24. The process according to claim 7,
wherein in the formula (7) and the formula (8),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

25. The process according to claim 7,
wherein in the formula (7) and the formula (8),
R¹ is methyl,
R² is trifluoromethyl,
R³ is difluoromethyl, and
R⁴ and R⁵ are methyl.

26. The process according to claim 13,
wherein in the formula (7) and the formula (8),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

27. The process according to claim 13,
wherein in the formula (7) and the formula (8),
R¹ is methyl,
R² is trifluoromethyl,
R³ is difluoromethyl, and
R⁴ and R⁵ are methyl.

28. The process according to claim 19,
wherein in the formula (7) and the formula (8),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

29. The process according to claim 19,
wherein in the formula (7) and the formula (8),
R¹ is methyl,
R² is trifluoromethyl,
R³ is difluoromethyl, and
R⁴ and R⁵ are methyl.
